SEQUENCE LISTING (42 sequences)

SEQ ID NO: 1 - protein, 625 aa, Homo sapiens [sequence omitted]

SEQ ID NO: 2 - DNA, 3278 bp, Homo sapiens
aggcacacaggcaaaatcaagttctacatctgtccctgtgtatgtcacttgtttgaatacgaaataaaattaaaaaaataaattcagtgtattgagaaagcaagcaattctctcaaggtatatttctgacatactaagattttaacgactttcacaaatatgctgtactgagagagaatgttacataacattgagaactagtacaagtaaatattaaagtgaagtgaccatttcctacacaagctcattcagaggaggatgaagaccattttggaggaagaaaagcacccttattaagaattgcagcaagtaagccaacaaggtcttttcaggatgattttcttatatcaagtggtacatttcattttatttacttcagtttctggtgaatgtgtgactcagttgttgaaggacacctgctttgaaggaggggacattactacggtcttcacaccaagcgccaagtactgccaggtagtctgcacttaccacccaagatgtttactcttcactttcacggcggaatcaccatctgaggatcccacccgatggtttacttgtgtcctgaaagacagtgttacagaaacactgccaagagtgaataggacagcagcgatttctgggtattctttcaagcaatgctcacaccaaataagcgcttgcaacaaagacatttatgtggacctagacatgaagggcataaactataacagctcagttgccaagagtgctcaagaatgccaagaaagatgcacggatgacgtccactgccactttttcacgtacgccacaaggcagtttcccagcctggagcatcgtaacatttgtctactgaagcacacccaaacagggacaccaaccagaataacgaagctcgataaagtggtgtctggattttcactgaaatcctgtgcactttctaatctggcttgtattagggacattttccctaatacggtgtttgcagacagcaacatcgacagtgtcatggctcccgatgcttttgtctgtggccgaatctgcactcatcatcccggttgcttgttttttaccttcttttcccaggaatggcccaaagaatctcaaagaaatctttgtctccttaaaacatctgagagtggattgcccagtacacgcattaaaaagagcaaagctctttctggtttcagtctacaaagctgcaggcacagcatcccagtgttctgccattcttcattttaccatgacactgatttcttgggagaagaactggatattgttgctgcaaaaagtcacgaggcctgccagaaactgtgcaccaatgccgtccgctgccagttttttacctataccccagcccaagcatcctgcaacgaagggaagggcaagtgttacttaaagctttcttcaaacggatctccaactaaaatacttcacgggagaggaggcatctctggatacacattaaggttgtgtaaaatggataatgagtgtaccaccaaaatcaagcccaggatcgttggaggaactgcgtctgttcgtggtgagtggccgtggcaggtgaccctgcacacaacctcacccactcagagacacctgtgtggaggctccatcattggaaaccagtggatattaacagccgctcactgtttctatggggtagagtcacctaagattttgcgtgtctacagtggcattttaaatcaatctgaaataaaagaggacacatctttctttggggttcaagaaataataatccatgatcagtataaaatggcagaaagcgggtatgatattgccttgttgaaactggaaaccacagtgaattacacagattctcaacgacccatatgcctgccttccaaaggagatagaaatgtaatatacactgattgctgggtgactggatgggggtacagaaaactaagagacaaaatacaaaatactctccagaaagccaagatacccttagtgaccaacgaagagtgccagaagagatacagaggacataaaataacccataagatgatctgtgccggctacagggaaggagggaaggacgcttgcaagggagattcgggaggccctctgtcctgcaaacacaatgaggtctggcatctggtaggcatcacgagctggggcgaaggctgtgctcaaagggagcggccaggtgtttacaccaacgtggtcgagtacgtggactggattctggagaaaactcaagcagtgtgaatgggttcccaggggccattggagtccctgaaggacccaggatttgctgggagagggtgttgagttcactgtgccagcatgcttcctccacagtaacacgctgaaggggcttggtgtttgtaagaaaatgctagaagaaaacaaactgtcacaagttgttatgtccaaaactcccgttctatgatcgttgtagtttgtttgagcattcagtctctttgtttttgatcacgcttctatggagtccaagaattaccataaggcaatatttctgaagattactatataggcagatatagcagaaaataaccaagtagtggcagtggggatcaggcagaagaactggtaaaagaagccaccataaatagatttgttcgatgaaagatgaaaactggaagaaaggagaacaaagacagtcttcaccattttgcaggaatctacactctgcctatgtgaacacatttcttttgtaaagaaagaaattgattgcatttaatggcagattttcagaatagtcaggaattcttgtcatttccattttaaaatatatattaaaaaaaatcagttcgagtagacacgagctaagagtgaatgtgaagataacagaatttctgtgtggaagaggattacaagcagcaatttacctggaagtgataccttaggggcaatcttgaagatacactttcctgaaaaatgatttgtgatggattgtatatttatttaaaatatcttgggaggggaggctgatggagatagggagcatgctcaaacctccctaagacaagctgctgctgtgactatgggctcccaaagagctagatcgtatatttatttgacaaaaatcaccatagactgcatccatactacagagaaaaaacaattagggcgcaaatggatagttacagtaaagtcttcagcaagcagctgcctgtattctaagcactgggattttctgtttcgtgcaaatatttatctcattattgttgtgatctagttcaataacctagaatttgaattgtcaccacatagctttcaatctgtgccaacaactatacaattcatcaagtgtg

SEQ ID NO: 3 - protein, 5 aa, Artificial Sequence
  Synthetic peptide
TAAMS

SEQ ID NO: 4 - protein, 17 aa, Artificial Sequence
  Synthetic peptide
GISGSGSSTYYADSVKG

SEQ ID NO: 5 - protein, 13 aa, Artificial Sequence
  Synthetic peptide
ELSYLYSGYYFDY

SEQ ID NO: 6 - protein, 7 aa, Artificial Sequence
  Synthetic peptide
GFTFSTA

SEQ ID NO: 7 - protein, 6 aa, Artificial Sequence
  Synthetic peptide
SGSGSS

SEQ ID NO: 8 - protein, 13 aa, Artificial Sequence
  Synthetic peptide
ELSYLYSGYYFDY

SEQ ID NO: 9 - protein, 122 aa, Artificial Sequence
  Synthetic peptide
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSS

SEQ ID NO: 10 - DNA, 366 bp, Artificial Sequence
  Synthetic peptide
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctca

SEQ ID NO: 11 - protein, 452 aa, Artificial Sequence
  Synthetic peptide
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 1356 bp, Artificial Sequence
  Synthetic peptide
caggtgcaattgctggaaagcggcggtggcctggtgcagccgggtggcagcctgcgtctgagctgcgcggcgtccggattcaccttttctactgctgctatgtcttgggtgcgccaggccccgggcaaaggtctcgagtgggtttccggtatctctggttctggttcttctacctactatgcggatagcgtgaaaggccgctttaccatcagccgcgataattcgaaaaacaccctgtatctgcaaatgaacagcctgcgtgcggaagatacggccgtgtattattgcgcgcgtgaactgtcttacctgtactctggttactacttcgattactggggccaaggcaccctggtgactgttagctcagcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaagcagcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 13 - protein, 13 aa, Artificial Sequence
  Synthetic peptide
SGSSSNIGSNDVS

SEQ ID NO: 14 - protein, 7 aa, Artificial Sequence
  Synthetic peptide
KNYNRPS

SEQ ID NO: 15 - protein, 11 aa, Artificial Sequence
  Synthetic peptide
SAWDQRQFDVV

SEQ ID NO: 16 - protein, 9 aa, Artificial Sequence
  Synthetic peptide
SSSNIGSND

SEQ ID NO: 17 - protein, 3 aa, Artificial Sequence
  Synthetic peptide
KNY

SEQ ID NO: 18 - protein, 8 aa, Artificial Sequence
  Synthetic peptide
WDQRQFDV

SEQ ID NO: 19 - protein, 110 aa, Artificial Sequence
  Synthetic peptide
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVL

SEQ ID NO: 20 - DNA, 330 bp, Artificial Sequence
  Synthetic peptide
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtccta

SEQ ID NO: 21 - protein, 216 aa, Artificial Sequence
  Synthetic peptide
DIVLTQPPSVSGAPGQRVTISCSGSSSNIGSNDVSWYQQLPGTAPKLLIYKNYNRPSGVPDRFSGSKSGTSASLAITGLQAEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - DNA, 648 bp, Artificial Sequence
  Synthetic peptide
gatatcgtgctgacccagccgccgagcgtgagcggtgcaccgggccagcgcgtgaccattagctgtagcggcagcagcagcaacattggttctaacgacgtgtcttggtaccagcagctgccgggcacggcgccgaaactgctgatctacaaaaactacaaccgcccgagcggcgtgccggatcgctttagcggatccaaaagcggcaccagcgccagcctggcgattaccggcctgcaagcagaagacgaagcggattattactgctctgcttgggaccagcgtcagttcgacgttgtgtttggcggcggcacgaagttaaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 23 - protein, 5 aa, Artificial Sequence
  Synthetic peptide
TAAMS

SEQ ID NO: 24 - protein, 17 aa, Artificial Sequence
  Synthetic peptide
GISGSGSSTYYADSVKG

SEQ ID NO: 25 - protein, 13 aa, Artificial Sequence
  Synthetic peptide
ELSYLYSGYYFDY

SEQ ID NO: 26 - protein, 7 aa, Artificial Sequence
  Synthetic peptide
GFTFSTA

SEQ ID NO: 27 - protein, 6 aa, Artificial Sequence
  Synthetic peptide
SGSGSS

SEQ ID NO: 28 - protein, 13 aa, Artificial Sequence
  Synthetic peptide
ELSYLYSGYYFDY

SEQ ID NO: 29 - protein, 122 aa, Artificial Sequence; identical to SEQ ID NO: 9
  Synthetic peptide

SEQ ID NO: 30 - DNA, 366 bp, Artificial Sequence
  Synthetic peptide
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagc

SEQ ID NO: 31 - protein, 452 aa, Artificial Sequence
  Synthetic peptide
QVQLLESGGGLVQPGGSLRLSCAASGFTFSTAAMSWVRQAPGKGLEWVSGISGSGSSTYYADSVKGRFTISRDNSKNTLYLQMNSLRAEDTAVYYCARELSYLYSGYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 1356 bp, Artificial Sequence
  Synthetic peptide
caggtgcagctgctggaatcaggcggcggactggtgcagcctggcggtagcctgagactgagctgcgctgctagtggcttcacctttagcaccgccgctatgagctgggttcgacaggccccagggaaaggcctcgagtgggtctcagggattagcggtagcggctctagcacctactacgccgatagcgtgaagggccggttcactatctctagggataactctaagaacaccctgtacctgcagatgaatagcctgagagccgaggacaccgccgtctactactgcgctagagagctgagctacctgtatagcggctactacttcgactactggggtcaaggcaccctggtcaccgtgtctagcgctagcactaagggcccctccgtgttccctctggccccttccagcaagtctacctccggcggcacagctgctctgggctgcctggtcaaggactacttccctgagcctgtgacagtgtcctggaactctggcgccctgacctctggcgtgcacaccttccctgccgtgctgcagtcctccggcctgtactccctgtcctccgtggtcacagtgccttcaagcagcctgggcacccagacctatatctgcaacgtgaaccacaagccttccaacaccaaggtggacaagcgggtggagcctaagtcctgcgacaagacccacacctgtcctccctgccctgctcctgaactgctgggcggcccttctgtgttcctgttccctccaaagcccaaggacaccctgatgatctcccggacccctgaagtgacctgcgtggtggtggccgtgtcccacgaggatcctgaagtgaagttcaattggtacgtggacggcgtggaggtgcacaacgccaagaccaagcctcgggaggaacagtacaactccacctaccgggtggtgtccgtgctgaccgtgctgcaccaggactggctgaacggcaaagagtacaagtgcaaagtctccaacaaggccctggccgcccctatcgaaaagacaatctccaaggccaagggccagcctagggaaccccaggtgtacaccctgccacccagccgggaggaaatgaccaagaaccaggtgtccctgacctgtctggtcaagggcttctacccttccgatatcgccgtggagtgggagtctaacggccagcctgagaacaactacaagaccacccctcctgtgctggactccgacggctccttcttcctgtactccaaactgaccgtggacaagtcccggtggcagcagggcaacgtgttctcctgctccgtgatgcacgaggccctgcacaaccactacacccagaagtccctgtccctgtctcccggcaag

SEQ ID NO: 33 - protein, 13 aa, Artificial Sequence
  Synthetic peptide
SGSSSNIGSNDVS

SEQ ID NO: 34 - protein, 7 aa, Artificial Sequence
  Synthetic peptide
KNYNRPS

SEQ ID NO: 35 - protein, 11 aa, Artificial Sequence
  Synthetic peptide
SAWDQRQFDVV

SEQ ID NO: 36 - protein, 9 aa, Artificial Sequence
  Synthetic peptide
SSSNIGSND

SEQ ID NO: 37 - protein, 3 aa, Artificial Sequence
  Synthetic peptide
KNY

SEQ ID NO: 38 - protein, 8 aa, Artificial Sequence
  Synthetic peptide
WDQRQFDV

SEQ ID NO: 39 - protein, 110 aa, Artificial Sequence
  Synthetic peptide
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVL

SEQ ID NO: 40 - DNA, 330 bp, Artificial Sequence
  Synthetic peptide
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctg

SEQ ID NO: 41 - protein, 216 aa, Artificial Sequence
  Synthetic peptide
QSVLTQPPSASGTPGQRVTISCSGSSSNIGSNDVSWYQQLPGTAPKLLIYKNYNRPSGVPDRFSGSKSGTSASLAISGLQSEDEADYYCSAWDQRQFDVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - DNA, 648 bp, Artificial Sequence
  Synthetic peptide
cagtcagtcctgactcagccccctagcgctagtggcacccctggtcaaagagtgactattagctgtagcggctctagctctaatatcggctctaacgacgtcagctggtatcagcagctgcccggcaccgcccctaagctgctgatctataagaactataataggcctagcggcgtgcccgataggtttagcggatctaaatcagggacttctgctagtctggctattagcggcctgcagtcagaggacgaggccgactactactgtagcgcctgggatcagcgtcagttcgacgtggtgttcggcggaggcactaagctgaccgtgctgggtcaacctaaggctgcccccagcgtgaccctgttcccccccagcagcgaggagctgcaggccaacaaggccaccctggtgtgcctgatcagcgacttctacccaggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacaggtcctacagctgccaggtgacccacgagggcagcaccgtggaaaagaccgtggccccaaccgagtgcagc

(19) United States
(12) Patent Application Publication
(10) Pub. No.: US 2023/0071973 A1
(43) Pub. Date: Mar. 9, 2023

(54) PHARMACEUTICAL FORMULATIONS AND DOSAGE REGIMENS FOR FACTOR XI/XIA ANTIBODIES

(71) Applicant: Anthos Therapeutics, Inc., Cambridge, MA (US)

(72) Inventors: Debra A. FREEDHOLM, Cambridge, MA (US); Daniel M. BLOOMFIELD, Cambridge, MA (US); Royston J. GLASSPOOL, Cambridge, MA (US); Jonathan E. FREEMAN, Cambridge, MA (US)

(21) Appl. No.: 17/785,660

(22) PCT Filed: Dec. 18, 2020
(86) PCT No.: PCT/US2020/066151

§ 371 Date: Jun. 15, 2022

Related U.S. Application Data
(60) Provisional application No. 62/951,887, filed on Dec. 20, 2019.

(51) Int. Cl. C07K 16/36; A61K 47/22; A61K 47/26; A61P 9/06; A61P 7/02; A61K 9/00

(52) U.S. Cl. CPC C07K 16/36; A61K 47/22; A61K 47/26; A61P 9/06; A61P 7/02; A61K 9/0019; C07K 2317/52

(57) ABSTRACT

This disclosure relates to pharmaceutical formulations of anti-Factor XI and/or activated Factor XI (Factor XIa) antibodies, or antigen-binding fragments thereof. Also provided are dosage regimens for such antibodies or antigen-binding fragments thereof, pharmaceutical formulations comprising the same, and pharmaceutical formulations for use in the treatment of thromboembolic disorders or related conditions.

### CROSS-REFERENCE TO RELATED APPLICATIONS

This application claims the benefit of and priority to U.S. Provisional Patent Application No. 62/951,887, filed on Dec. 20, 2019, the disclosure of which is hereby incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Dec. 18, 2020, is named ATD-008WO_ST25.txt and is 39,163 bytes in size.

### FIELD OF THE DISCLOSURE

The present disclosure relates generally to pharmaceutical formulations of anti-Factor XI and/or activated Factor XI (Factor XIa) antibodies, or antigen-binding fragments thereof; it also relates generally to dosage regimens for such antibodies or antigen-binding fragments thereof, or pharmaceutical formulations comprising the same; and pharmaceutical formulations for use in the treatment of thromboembolic disorders or related conditions.

### BACKGROUND

There exists a high unmet medical need for safer therapies to reduce thromboembolic complications such as stroke, systemic embolism, cognitive decline and mortality, with comparable or improved efficacy to that exhibited by existing therapies, and with a lower risk of bleeding.

Factor XI (FXI) is a serine protease functioning both in the intrinsic and extrinsic coagulation pathways. Factor XI exists in the zymogen form as a homodimer; upon cleavage of the peptide bond at R369-1370, Factor XI is activated (Factor XIa, FXIa). FXI plays a minor role in normal hemostasis in a high tissue factor environment but does play a key role in thrombosis. Genetic Factor XI deficiency is associated with decreased incidence of ischemic stroke and venous thromboembolic events (Salomon et al. (2008); Salomon, et al. (2011) Thromb Haemost.; 105:269-73). Bleeding manifestations in subjects with Factor XI deficiency are infrequent, often mild, result from injury or trauma, and very rarely affect critical organs (Salomon et al. (2011)).

Antibodies that bind Factor XI and/or Factor XIa have been studied. For example, WO 2016/207858 describes one such anti-Factor XI and/or Factor XIa antibody, disclosed in Table 1 as Antibody 1. The present disclosure adds to these developments and provides further clinical methods, including dosage regimens, to treat patients with specific thromboembolic disorders with desired safety and efficacy. Furthermore, the present disclosure adds to the earlier developments in the field by providing formulations comprising such FXI and/or FXIa antibodies that are sufficiently stable and suitable for administration to patients.

### SUMMARY OF THE DISCLOSURE

The present disclosure relates to pharmaceutical formulations for anti-Factor XI and/or activated Factor XI (Factor XIa) antibodies, or antigen-binding fragments thereof. Also provided are dosage regimens for such antibodies or antigen-binding fragments thereof, and pharmaceutical formulations for use in the treatment of thromboembolic disorders or related conditions.

Accordingly, in one aspect, provided herein is a vial comprising a drug delivery formulation comprising: (a) a therapeutically effective amount of an isolated anti-Factor XI (FXI) and/or anti-activated Factor XI (FXIa) antibody, or antigen-binding fragment thereof; (b) a histidine buffer; (c) a sugar or sugar alcohol; and (d) a polysorbate, at pH 5.0 to 6.0, wherein the vial comprises an overfill for complete withdrawal of a therapeutically effective amount of the anti-FXI and/or anti-FXIa antibody or the antigen-binding fragment thereof.

In certain embodiments, the vial comprises the therapeutically effective amount of the isolated anti-FXI and/or anti-FXIa antibody or antigen-binding fragment thereof at a concentration between 120 mg/ml and 180 mg/ml. In certain embodiments, the vial comprises the therapeutically effective amount of the isolated anti-FXI and/or anti-FXIa antibody or antigen-binding fragment thereof at a concentration of about 150 mg/ml.

In certain embodiments, the histidine buffer comprises a histidine and a histidine salt. In certain embodiments, the histidine is L-histidine. In certain embodiments, the histidine salt is histidine HCl monohydrate. In certain embodiments, the histidine buffer is at a concentration between 10 mM and 30 mM. In certain embodiments, the histidine buffer is at a concentration of about 20 mM.

In certain embodiments, the sugar or sugar alcohol is a disaccharide. In certain embodiments, the disaccharide is sucrose. In certain embodiments, the sucrose is at a concentration between 170 mM to 270 mM. In certain embodiments, the sucrose is at a concentration of about 220 mM.

In certain embodiments, the polysorbate is polysorbate 20. In certain embodiments, the polysorbate 20 is at a concentration between 0.02% (v/v) to 0.06% (v/v). In certain embodiments, the polysorbate 20 is at a concentration of about 0.04% (v/v).

In certain embodiments, the pH of the drug delivery formulation is 5.3 to 5.7. In certain embodiments, the pH of the drug delivery formulation is about 5.5.

In certain embodiments, the overfill comprises between 10% (v/v) and 30% (v/v) of the drug delivery formulation, optionally wherein the vial comprises 1.1 mL to 1.3 mL of the drug delivery formulation. In certain embodiments, the overfill comprises about 20% (v/v) of the drug delivery formulation, optionally wherein the vial comprises about 1.2 mL of the drug delivery formulation.

In another aspect, disclosed herein is a vial comprising a drug delivery formulation comprising:

(a) a therapeutically effective amount of an isolated anti-Factor XI (FXI) and/or anti-activated Factor XI (FXIa) antibody, or antigen-binding fragment thereof at a concentration of about 150 mg;

(b) a histidine buffer at a concentration of about 20 mM;

(c) sucrose at a concentration of about 220 mM; and

(d) polysorbate-20 at a concentration of about 0.04% (v/v),

at pH 5.5,

wherein the vial comprises an overfill for complete withdrawal of a therapeutically effective amount of the anti-FXI and/or anti-FXIa antibody or the antigen-binding fragment thereof.

In another aspect, provided herein is an intravenous drug delivery formulation comprising: (a) a therapeutically effective amount of an isolated anti-FXI and/or anti-FXIa antibody or antigen-binding fragment thereof; (b) a histidine buffer; (c) a sugar or sugar alcohol; (d) a polysorbate, and (e) a diluent, at pH 5.0 to 6.0, wherein the diluent is a solution comprising a second sugar and water.

In certain embodiments, the therapeutically effective amount of the isolated anti-FXI and/or anti-FXIa antibody or antigen-binding fragment thereof is at a concentration between 1.20 mg/ml and 1.80 mg/ml. In certain embodiments, the therapeutically effective amount of the isolated anti-FXI and/or anti-FXIa antibody or antigen-binding fragment thereof is at a concentration of about 1.50 mg/ml.

In certain embodiments, the histidine buffer comprises a histidine and a histidine salt. In certain embodiments, the histidine is L-histidine. In certain embodiments, the histidine salt is histidine HCl monohydrate. In certain embodiments, the histidine buffer is at a concentration between 0.10 mM and 0.30 mM. In certain embodiments, the histidine buffer is at a concentration of about 0.20 mM.

In certain embodiments, the sugar or sugar alcohol is a disaccharide. In certain embodiments, the disaccharide is sucrose. In certain embodiments, the sucrose is at a concentration between 1.70 mM to 2.70 mM. In certain embodiments, the sucrose is at a concentration of about 2.20 mM.

In certain embodiments, the polysorbate is polysorbate 20. In certain embodiments, the polysorbate 20 is at a concentration of less than 0.001% (v/v). In certain embodiments, the polysorbate 20 is at a concentration of about 0.0004% (v/v).

In certain embodiments, the pH of the intravenous drug delivery formulation is 5.3 to 5.7. In certain embodiments, the pH of the intravenous drug delivery formulation is about 5.5.

In certain embodiments, the second sugar in the diluent is a monosaccharide. In certain embodiments, the monosaccharide is dextrose. In certain embodiments, the dextrose is at a concentration between 2.5% (v/v) and 7.5% (v/v). In certain embodiments, the dextrose is at a concentration of about 5% (v/v).

In another aspect, disclosed herein is an intravenous drug delivery formulation comprising:
(a) a therapeutically effective amount of an isolated anti-FXI and/or anti-FXIa antibody or antigen-binding fragment thereof at a concentration of about 1.5 mg;
(b) a histidine buffer at a concentration of about 0.20 mM;
(c) sucrose at a concentration of about 2.20 mM;
(d) a polysorbate-20 at a concentration of about 0.0004% (v/v), and
(e) a diluent at pH 5.5, wherein the diluent is dextrose 5% in water (D5W).

The following features can be incorporated into any of the embodiments recited above:

In certain embodiments, the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

In certain embodiments, the antibody or antigen-binding fragment thereof comprises: i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35; ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37; and a light chain variable region CDR3 of SEQ ID NO: 38; iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37; and a light chain variable region CDR3 of SEQ ID NO: 15; or iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

In certain embodiments, the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto. In certain embodiments, the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

In certain embodiments, the antibody or antigen-binding fragment thereof comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

In certain embodiments, the antibody or antigen-binding fragment thereof comprises a heavy chain comprising an amino acid sequence of SEQ ID NO: 31 and a light chain comprising an amino acid sequence of SEQ ID NO: 41.

In certain embodiments, the antibody is a human monoclonal antibody. In certain embodiments, the antibody is a human IgG1 isotype. In certain embodiments, the antibody comprises D265A and P329A substitutions in the Fc domain, optionally wherein 120 mg to 180 mg is the therapeutically effective amount of the anti-Factor XI (FXI) and/or anti-activated Factor XI (FXIa) antibody or antigen-binding fragment thereof, for administration to a subject.

In another aspect, provided herein is a method of treating a subject afflicted with or at risk of developing a thromboembolic disorder, the method comprising administering a therapeutically effective amount of the drug delivery formulation present in the vial or the intravenous drug delivery formulation to the subject in need thereof.

In certain embodiments, the thromboembolic disorder is selected from the group consisting of atrial fibrillation or atrial flutter, transient ischemic attack, ischemic stroke, thromboembolic stroke, hemorrhagic stroke, venous thromboembolism (VTE), pediatric VTE, systemic embolism, non-central nervous systemic embolism, myocardial infarction, deep vein thrombosis, Severe Protein S deficiency, cerebrovascular accident, and cancer.

In certain embodiments, the drug delivery formulation present in the vial or the intravenous drug delivery formulation is administered monthly.

In certain embodiments, the drug delivery formulation present in the vial or the intravenous drug delivery formulation is administered at a dose selected from the group consisting of about 30 mg, about 60 mg, about 90 mg, about 120 mg, about 150 mg, and about 180 mg. In certain embodiments, the drug delivery formulation present in the vial is administered at a dose of about 90 mg. In certain embodiments, the drug delivery formulation present in the vial is administered at a dose of about 120 mg. In certain embodiments, the drug delivery formulation present in the vial is administered at a dose of about 150 mg.

In certain embodiments, the drug delivery formulation present in the vial is administered subcutaneously.

In certain embodiments, wherein the thromboembolic disorder is atrial fibrillation or atrial flutter. In certain embodiments, wherein the atrial fibrillation or atrial flutter is paroxysmal atrial fibrillation (PAF).

In certain embodiments, the drug delivery formulation present in the vial is administered once a month for a period of three months.

In certain embodiments, wherein the subject is at low risk of stroke. In certain embodiments, the subject has a CHA₂DS₂VASc risk score of 0 to 1.

In certain embodiments, the subject is at moderate risk of stroke.

In certain embodiments, the subject is at high risk of stroke. In certain embodiments, the subject has a CHA₂DS₂VASc risk score of ≥2 for male subjects and >3 for female subjects.

In certain embodiments, the method further comprises evaluating efficacy of the drug delivery formulation present in the vial by measuring inhibition of Factor XI at the trough after the third dose of the drug delivery formulation. In certain embodiments, the method further comprises evaluating efficacy of the drug delivery formulation present in the vial by assessing one or more biomarkers selected from the list consisting of free Factor XI, total Factor XI, Factor XI coagulation activity, activated partial thromboplastin time, and D-dimer.

In certain embodiments, the method further comprises evaluating adverse events to the drug delivery formulation present in the vial by measuring bleeding events or the presence of anti-drug antibodies. In certain embodiments, the method further comprises applying one or more of the following to the patient experiencing an adverse event, wherein the adverse event is a bleeding event: (i) fluid replacement using colloids, crystalloids, human plasma or plasma proteins such as albumin; (ii) transfusion with packed red blood or whole blood; or (iii) administration of fresh frozen plasma (FFP), prothrombin complex concentrates (PCC), activated PCC (APCC), such as, factor VIII inhibitor, and/or recombinant, activated factor VII.

In another aspect, provided herein is a method of treating a subject afflicted with or at risk of developing a thromboembolic disorder and who is undergoing a surgical procedure, the method comprising administering the intravenous drug delivery formulation of any one of claims 29-58 to a subject in need thereof, wherein the subject is administered the intravenous drug delivery formulation on the same day as the surgical procedure.

In certain embodiments, the surgical procedure is selected from the group consisting of knee replacement surgery, e.g., unilateral total knee arthroplasty (TKA), hip replacement surgery, orthopedic surgery, pacemaker installation, catheter installation, thoracic surgery, and abdominal surgery. In certain embodiments, the surgical procedure is unilateral total knee arthroplasty (TKA).

In certain embodiments, the intravenous drug delivery formulation is administered monthly.

In certain embodiments, the intravenous drug delivery formulation is administered at a dose selected from the group consisting of about 30 mg, about 60 mg, about 90 mg, about 120 mg, about 150 mg, and about 180 mg. In certain embodiments, the intravenous drug delivery formulation is administered at a dose of about 30 mg. In certain embodiments, the intravenous drug delivery formulation is administered at a dose of about 60 mg. In certain embodiments, the intravenous drug delivery formulation is administered at a dose of about 75 mg. In certain embodiments, the intravenous drug delivery formulation is administered at a dose of about 150 mg.

In certain embodiments, the intravenous drug delivery is administered approximately 4-8 hours after surgery.

In another aspect, provided herein is a method of treating a subject afflicted with or at risk of developing a thromboembolic disorder, wherein the subject is receiving non-steroidal anti-inflammatory drugs (NSAIDs), the method comprising administering a therapeutically effective amount of the drug delivery formulation present in the vial or the intravenous drug delivery in combination with a proton-pump inhibitor to the subject in need thereof.

In certain embodiments, the drug delivery formulation present in the vial or the intravenous drug delivery formulation is administered monthly.

In certain embodiments, the drug delivery formulation present in the vial or the intravenous drug delivery formulation is administered at a dose selected from the group consisting of about 30 mg, about 60 mg, about 90 mg, about 120 mg, about 150 mg, and about 180 mg.

In certain embodiments, the drug delivery formulation present in the vial is administered subcutaneously.

Other embodiments and details of the disclosure are presented herein below.

### BRIEF DESCRIPTION OF THE DRAWINGS

FIG. 1A-FIG. 1B show linearity analysis of size exclusion high-performance liquid chromatography (SE-HPLC) for drug product containing antibody 1 diluted in dextrose (D5W, shown in FIG. 1A) or aprotinin (APN, shown in FIG. 1B).

FIG. 2A-FIG. 2C show the temporal duration of the effects of Antibody 1 on various biomarkers. FIG. 2A shows the concentration of Antibody 1 in plasma for subjects with atrial fibrillation (AF). FIG. 2B shows free Factor XI in plasma for AF subjects treated with Antibody 1. FIG. 2C demonstrates the effect of multiple doses of Antibody 1 on free Factor XI.

FIG. 3A-FIG. 3C show safety analysis of experimental antithrombotic treatments based on number of patients experiencing any venous thromboembolism (VTE) event for Antibody 1 (FIG. 3A), and published clinical trials with Factor XI antisense oligonucleotide (FXI-ASO) (FIG. 3B), and FOXTROT (FIG. 3C); each experimental treatment shows comparison to enoxaparin as a control.

FIG. 4A-FIG. 4B show the temporal duration of the effects of Antibody 1 on various biomarkers. FIG. 4A shows the concentration of Antibody 1 in plasma for subjects undergoing total knee arthroplasty (TKA). FIG. 4B shows free Factor XI in plasma for TKA subjects treated with Antibody 1.

### DETAILED DESCRIPTION

### Definitions

To facilitate an understanding of the present invention, a number of terms and phrases are defined below.

The terms “a” and “an” as used herein mean “one or more” and include the plural unless the context is inappropriate.

As used herein, the terms “FXI protein,” “FXI antigen,” and “FXI” are used interchangeably, and refers to the Factor XI protein in different species. Factor XI is the mammalian plasma coagulation factor XI, a glycoprotein present in human plasma at a concentration of 25-30 nM as a zymogen that when converted by limited proteolysis to an active serine protease, participates in the intrinsic pathway of blood coagulation.

The terms “FXIa protein,” “FXIa antigen,” and “FXIa”, are used interchangeably, and refers to the activated FXI protein in different species. The zymogen Factor XI is converted into its active form, the coagulation factor Xla (FXIa), either via the contact phase of blood coagulation or through thrombin-mediated activation on the platelet surface. During this activation of factor XI, an internal peptide bond is cleaved in each of the two chains, resulting in the activated factor Xla, a serine protease composed of two heavy and two light chains held together by disulfide bonds. This serine protease FXIa converts the coagulation Factor IX into IXa, which subsequently activates coagulation Factor X (Xa). Xa then can mediate coagulation Factor 11/Thrombin activation. For example, human FXI has the sequence as set out in Table 1 (SEQ ID NO:1) and has been described in previous reports and literature (Mandle R J Jr, et al. (1979) Blood; 54(4):850; NCBI Reference Sequence: AAA51985).

In the context of this present disclosure, the terms “FXI” and “FXIa” (and the like) include mutants and variants of the natural FXI and FXIa protein, respectively, which have substantially the same amino acid sequence as that of the native primary structure (amino acid sequence) described in the above-mentioned reports.

The term “catalytic domain,” “serine protease catalytic domain,” and similar terms as used herein, means amino acids Ile370 to Va607, as counted from the Glu1 at the N-terminus of the mature protein that is in circulation. It can also be described as residues 388-625 at the C-terminus of FXI. As used herein, the term “active site” means the catalytic triad comprised of the amino acids His413, Asp462 and Ser557. (Bane and Gailani (2014) Drug Disc. 19(9)).

The term “antibody” as used herein means a whole antibody and any antigen binding fragment (e.g., “antigen-binding portion”) or single chain thereof. A whole antibody is a glycoprotein comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1 CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. In some specific aspects, an antibody can be a monoclonal antibody, human antibody, humanized antibody, camelised antibody, or chimeric antibody. Antibodies can be of any isotype (e.g., IgG, IgE, IgM, IgD, IgA and IgY), class (e.g., IgG1, IgG2, IgG3, IgG4, IgA1 and IgA2) or subclass.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

As used herein, the terms “drug delivery formulation” or “intravenous drug delivery formulation” refers to a pharmaceutical formulation comprising the combination of an active agent with a carrier, inert or active, making the composition especially suitable for diagnostic or therapeutic use in vivo or ex vivo.

As used herein, the terms “subject” and “patient” refer to an organism to be treated by the methods and compositions described herein. Such organisms preferably include, but are not limited to, mammals (e.g., murines, simians, equines, bovines, porcines, primates, canines, felines, and the like), and more preferably include humans. In certain embodiments, the subject is a human.

A “thromboembolic disorder,” or similar terms as used herein, refer to any number of conditions or diseases in which the intrinsic and/or common coagulation pathways are aberrantly activated or are not naturally deactivated (e.g., without therapeutic means). These conditions include but are not limited to thromboemolic stroke and other types of stroke of ischemicorigin, atrial fibrillation, stroke prevention in atrial fibrillation (SPAF), deep vein thrombosis, venous thromboembolism, and pulmonary embolism. These can also include prevention and treatment of catheter-related thromobsis (e.g., Hickman catheter in oncology patients) in which catheters become thrombosed, and extracorporeal membrane oxygenation (ECMO), in which the tubing and oxygenation membrane develops clots.

A “thromboembolic disorder” or similar terms as used herein, can also refer to any number of the following, which the anti-FXI and/or FXIa antibodies or antigen binding fragments thereof of the present disclosure can be used to prevent or treat:
thromboembolism in subjects with suspected or confirmed cardiac arrhythmia such as paroxysmal, persistent or permanent atrial fibrillation or atrial flutter;
stroke prevention in atrial fibrillation (SPAF), a subpopulation of which is AF patients undergoing percutaneous coronary interventions (PCI);
acute venous thromboembolic events (VTE) treatment and extended secondary VTE prevention in patients at high risk for bleeding;
venous thromboembolism, wherein the subject is a pediatric subject (pediatric VTE);
cerebral and cardiovascular events in secondary prevention after transient ischemic attack (TIA) or non-disabling stroke and prevention of thromboembolic events in heart failure with sinus rhythm;
hemorrhagic stroke;
clot formation in left atrium and thromboembolism in subjects undergoing cardioversion for cardiac arrhythmia;
thrombosis before, during and after ablation procedure for cardiac arrhythmia;
venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins;
thrombosis on any artificial surface in the veins or arteries like catheter, pacemaker wires, synthetic arterial grafts; mechanical or biological heart valves or left ventricular assist device;
pulmonary embolism in patients with or without venous thrombosis;
Chronic Thromboembolic Pulmonary Hypertension (CTEPH);
arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension;
thrombosis and thromboembolism in patients undergoing percutaneous coronary interventions (PCI);
cardioembolic and cryptogenic strokes;
non-central nervous systemic embolism (non-CNS systemic embolism);
thrombosis in patients with invasive and non-invasive cancer malignancies;
thrombosis over an indwelling catheter;
thrombosis and thromboembolism in severely ill patients;
cardiac thrombosis and thromboembolism, including but not limited to cardiac thrombosis after myocardial infarction, cardiac thrombosis related to condition such as cardiac aneurysm, myocardial fibrosis, cardiac enlargement and insufficiency, myocarditis and artificial surface in the heart;
thromboembolism in patients with valvular heart disease with or without atrial fibrillation;
thromboembolism over valvular mechanic or biologic prostheses;
thromboembolism in patients who had native or artificial cardiac patches, arterial or venous conduit tubes after heart repair of simple or complex cardiac malformations;
venous thrombosis and thromboembolism after knee replacement surgery, hip replacement surgery, and orthopedic surgery, thoracic or abdominal surgery;
arterial or venous thrombosis after neurosurgery including intracranial and spinal cord interventions;
congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia;
thrombosis and thromboembolism in chronic kidney disease; and
thrombosis and thromboembolism in patients undergoing hemodialysis and in patients undergoing extra-corporal membrane oxygenation.

As used herein, the term “trough” or “trough level” refers to the lowest concentration reached by a drug before the next dose of the drug is administered. In certain embodiments, inhibition of Factor XI/Factor XIa at trough is greater than about 50% (e.g., greater than about 60%, greater than about 70%, greater than about 80%, or greater than about 90%). In certain embodiments, inhibition of Factor XI/Factor XIa at trough is greater than about 80%. In certain embodiments, inhibition of Factor XI/Factor XIa at trough is greater than about 90%.

The terms “treat,” “treating,” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By “therapeutic benefit,” what is meant is eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

In certain embodiments of the methods described herein, the subject is treatment naive, i.e., has never received any form of anticoagulant therapy prior to treatment with an anti-Factor XI/XIa antibody described herein, e.g., Antibody 1. In certain embodiments of the methods described herein, the subject has received a stable treatment of a recommended dose of a new oral anticoagulant (NOAC), e.g., prior to treatment with an anti-Factor XI/XIa antibody described herein, e.g., Antibody 1. In certain embodiments, the subject has received a direct oral anticoagulant (DOAC) e.g., prior to treatment with an anti-Factor XI/XIa antibody described herein, e.g., Antibody 1. In certain embodiments, the subject has received a Vitamin K antagonist (VKA) e.g., prior to treatment with an anti-Factor XI/XIa antibody described herein, e.g., Antibody 1.

As used herein, the term “vial” refers to a container that holds the drug product. In some embodiments, the vial may be a vial, a bag, a pen, or a syringe. In some embodiments, the vial may be a vial, e.g., a glass vial.

As used herein, the term “drug product” refers to an anti-Factor XI/XIa antibody described herein, e.g., Antibody 1 as disclosed in Table 1, and excipients, e.g., a histidine buffer, a sugar, and a polysorbate.

The term “about” refers to any minimal alteration in the concentration or amount of an agent that does not change the efficacy of the agent in preparation of a formulation and in treatment of a disease or disorder. In certain embodiments, the term “about” may include ±5%, ±10%, or ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

Anti-Factor XI and/or Activated Factor XI (Factor XIa) Antibodies

In some embodiments, the present disclosure provides pharmaceutical formulations comprising antibodies that bind FXI and/or FXIa protein (e.g., human, rabbit, cynomolgus monkey, and baboon FXI and/or FXIa), wherein the antibodies comprise a heavy chain variable domain (VH) having an amino acid sequence of SEQ ID NOs: 9 or 29, the formulations comprise a histidine buffer; a sugar or sugar alcohol; and a polysorbate, and the pH of the formulation is between pH 5.0 to 6.0. In certain embodiments, the antibodies comprise a VH having an amino acid sequence of SEQ ID NO:29.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

The present disclosure also provides a pharmaceutical formulations of antibodies that specifically bind to a FXI and/or FXIa protein, wherein the antibodies comprise a VH CDR having an amino acid sequence of any one of the VH CDRs listed in Table 1, infra, the formulations comprise a histidine buffer; a sugar or sugar alcohol; and a polysorbate; and the pH of the formulation is between pH 5.0 to 6.0. In particular, the present disclosure provides pharmaceutical formulations of antibodies that specifically bind to a FXI and/or FXIa protein (e.g., human, rabbit, cynomolgus monkey, and baboon FXI and/or FXIa), wherein the antibodies comprise (or alternatively, consist of) one, two, three, or more VH CDRs having an amino acid sequence of any of the VH CDRs listed in Table 1, infra, the formulations comprise a histidine buffer; a sugar or sugar alcohol; and a polysorbate; and the pH of the formulation is between pH 5.0 to 6.0. (see PCT International Patent Application No. PCT/IB2016/053790 filed on Jun. 24, 2016, and published as WO2016/207858, which is hereby incorporated by reference in its entirety).

In some embodiments, the present disclosure provides pharmaceutical formulations of antibodies that specifically bind to a FXIa protein, said antibodies comprising a light chain variable domain (VL) having an amino acid sequence of SEQ ID NOs: 19 or 39, for use in the methods described herein (e.g., methods for treating a subject afflicted with or at risk of developing a thromboembolic disorder), the formulations comprise a histidine buffer; a sugar or sugar alcohol; and a polysorbate; and the pH of the formulation is between pH 5.0 to 6.0. In certain embodiments, the antibodies comprise a VL having an amino acid sequence of SEQ ID NO:39.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

The present disclosure also provides pharmaceutical formulations of antibodies that specifically bind to a FXI and/or FXIa protein (e.g., human, rabbit, cynomolgus monkey, and baboon FXI and/or FXIa), for use in the methods described herein (e.g., methods for treating a subject afflicted with or at risk of developing a thromboembolic disorder), the antibodies comprising a VL CDR having an amino acid sequence of any one of the VL CDRs listed in Table 1, infra; the formulations comprise a histidine buffer; a sugar or sugar alcohol; and a polysorbate; and the pH of the formulation is between pH 5.0 to 6.0. The antibodies that specifically bind to an FXIa protein (e.g., human, rabbit, cynomolgus monkey, and baboon FXI and/or FXIa), may comprise (or alternatively, consist of) one, two, three or more VL CDRs having an amino acid sequence of any of the VL CDRs listed in Table 1, infra.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NO: 29, and a light chain variable domain (VL) having an amino acid sequence of SEQ ID NO: 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NO: 29, and a light chain variable domain (VL) having an amino acid sequence of SEQ ID NO: 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

In some embodiments, other antibodies for use in the methods described herein (e.g., methods for treating a subject afflicted with or at risk of developing a thromboembolic disorder) include amino acids that have been mutated, yet have at least 60, 70, 80, 85, 90 or 95 percent identity in the CDR regions with the CDR regions depicted in the sequences described in Table 1. In some embodiments, the antibodies include mutant amino acid sequences wherein no more than 1, 2, 3, 4 or 5 amino acids have been mutated in the CDR regions when compared with the CDR regions depicted in the sequence described in Table 1.

TABLE 1
Examples of FXI/FXIa Antibodies, Fabs and
FXI/FXIa Proteins

| | Sequence | |
|---|---|---|
| | Identifier | |
| Sequence | (SEQ ID | Amino acid or |
| Description | NO:) | polynucleotide sequence |
| Human FXIa full- | 1 | MIFLYQVVHFILFTSVSGECVTQLLKDTCFEGG |
| length protein | | DITTVFTPSAKYCQVVCTYHPRCLLFTFTAESP |
| sequence (NCBI | | SEDPTRWFTCVLKDSVTETLPRVNRTAAISGYS |
| Reference Sequence: | | FKQCSHQISACNKDIYVDLDMKGINYNSSVANS |
| AAA51985) | | AQECQERCTDDVHCHFFTYATRQFPSLEHRNIC |
| | | LLKHTQTGTPTRITKLDKVVSGFSLKSCALSNL |
| | | ACIRDIFPNTVFADSNIDSVMAPDAFVSGRICT |
| | | HHPGCLFFTFFSQEWPKESQRNLCLLKTSESGL |
| | | PSTRIKKSKALSGFSLQSCRHSIPVFCHSSFYH |
| | | DTDFLGEELDIVAAKSHEACQKLCTNAVRCQFF |
| | | TYTPAQASCNEGKGKCYLKLSSNGSPTKILHGR |
| | | GGISGYTLRLCKMDNECTTKIKPRIVGGTASVR |
| | | GEWPWQVTLHTTSPTQRHLCGGSIIGNQWILTA |
| | | AHCFYGVESPKILRVYSGILNQSEIKEDTSFFG |
| | | VQEIIIHDQYKMAESGYDIALLKLETTVNYTDS |
| | | QRPICLPSKGDRNVIYTDCWVTGWGYRKLRDKI |
| | | QNTLQKAKIPLVTNEECQKRYRGHKITHKMICA |
| | | GYREGGKDACKGDSGGPLSCKHNEVWHLVGITS |
| | | WGEGCAQRERPGVYTNVVEYVDWILEKTQAV |
| Human FXIa full- | 2 | AGGCACACAGGCAAAATCAAGTTCTACATCTGT |
| length nucleotide | | CCCTGTGTATGTCACTTGTTTGAATACGAAATA |
| sequence (NCBI | | AAATTAAAAAAATAAATTCAGTGTATTGAGAAA |
| Reference Sequence: | | GCAAGCAATTCTCTCAAGGTATATTTCTGACAT |
| NM_000128.3) | | ACTAAGATTTTAACGACTTTCACAAATATGCTG |
| | | TACTGAGAGAGAATGTTACATAACATTGAGAAC |
| | | TAGTACAAGTAAATATTAAAGTGAAGTGACCAT |
| | | TTCCTACACAAGCTCATTCAGAGGAGGATGAAG |
| | | ACCATTTTGGAGGAAGAAAAGCACCCTTATTAA |
| | | GAATTGCAGCAAGTAAGCCAACAAGGTCTTTTC |
| | | AGGATGATTTTCTTATATCAAGTGGTACATTTC |
| | | ATTTTATTTACTTCAGTTTCTGGTGAATGTGTG |
| | | ACTCAGTTGTTGAAGGACACCTGCTTTGAAGGA |
| | | GGGGACATTACTACGGTCTTCACACCAAGCGCC |
| | | AAGTACTGCCAGGTAGTCTGCACTTACCACCCA |
| | | AGATGTTTACTCTTCACTTTCACGGCGGAATCA |
| | | CCATCTGAGGATCCCACCCGATGGTTTACTTGT |
| | | GTCCTGAAAGACAGTGTTACAGAAACACTGCCA |
| | | AGAGTGAATAGGACAGCAGCGATTTCTGGGTAT |
| | | TCTTTCAAGCAATGCTCACACCAAATAAGCGCT |
| | | TGCAACAAAGACATTTATGTGGACCTAGACATG |
| | | AAGGGCATAAACTATAACAGCTCAGTTGCCAAG |
| | | AGTGCTCAAGAATGCCAAGAAAGATGCACGGAT |
| | | GACGTCCACTGCCACTTTTTCACGTACGCCACA |
| | | AGGCAGTTTCCCAGCCTGGAGCATCGTAACATT |
| | | TGTCTACTGAAGCACACCCAAACAGGGACACCA |
| | | ACCAGAATAACGAAGCTCGATAAAGTGGTGTCT |
| | | GGATTTTCACTGAAATCCTGTGCACTTTCTAAT |
| | | CTGGCTTGTATTAGGGACATTTTCCCTAATACG |
| | | GTGTTTGCAGACAGCAACATCGACAGTGTCATG |
| | | GCTCCCGATGCTTTTGTCTGTGGCCGAATCTGC |
| | | ACTCATCATCCCGGTTGCTTGTTTTTTACCTTC |
| | | TTTTCCCAGGAATGGCCCAAAGAATCTCAAAGA |
| | | AATCTTTGTCTCCTTAAAACATCTGAGAGTGGA |
| | | TTGCCCAGTACACGCATTAAAAAGAGCAAAGCT |
| | | CTTTCTGGTTTCAGTCTACAAAGCTGCAGGCAC |
| | | AGCATCCCAGTGTTCTGCCATTCTTCATTTTAC |
| | | CATGACACTGATTTCTTGGGAGAAGAACTGGAT |
| | | ATTGTTGCTGCAAAAAGTCACGAGGCCTGCCAG |
| | | AAACTGTGCACCAATGCCGTCCGCTGCCAGTTT |
| | | TTTACCTATACCCCAGCCCAAGCATCCTGCAAC |
| | | GAAGGGAAGGGCAAGTGTTACTTAAAGCTTTCT |
| | | TCAAACGGATCTCCAACTAAAATACTTCACGGG |
| | | AGAGGAGGCATCTCTGGATACACATTAAGGTTG |
| | | TGTAAAATGGATAATGAGTGTACCACCAAAATC |
| | | AAGCCCAGGATCGTTGGAGGAACTGCGTCTGTT |
| | | CGTGGTGAGTGGCCGTGGCAGGTGACCCTGCAC |
| | | ACAACCTCACCCACTCAGAGACACCTGTGTGGA |
| | | GGCTCCATCATTGGAAACCAGTGGATATTAACA |
| | | GCCGCTCACTGTTTCTATGGGGTAGAGTCACCT |
| | | AAGATTTTGCGTGTCTACAGTGGCATTTTAAAT |
| | | CAATCTGAAATAAAAGAGGACACATCTTTCTTT |
| | | GGGGTTCAAGAAATAATAATCCATGATCAGTAT |
| | | AAAATGGCAGAAAGCGGGTATGATATTGCCTTG |
| | | TTGAAACTGGAAACCACAGTGAATTACACAGAT |
| | | TCTCAACGACCCATATGCCTGCCTTCCAAAGGA |
| | | GATAGAAATGTAATATACACTGATTGCTGGGTG |
| | | ACTGGATGGGGGTACAGAAAACTAAGAGACAAA |
| | | ATACAAAATACTCTCCAGAAAGCCAAGATACCC |
| | | TTAGTGACCAACGAAGAGTGCCAGAAGAGATAC |
| | | AGAGGACATAAAATAACCCATAAGATGATCTGT |
| | | GCCGGCTACAGGGAAGGAGGGAAGGACGCTTGC |
| | | AAGGGAGATTCGGGAGGCCCTCTGTCCTGCAAA |
| | | CACAATGAGGTCTGGCATCTGGTAGGCATCACG |
| | | AGCTGGGGCGAAGGCTGTGCTCAAAGGGAGCGG |
| | | CCAGGTGTTTACACCAACGTGGTCGAGTACGTG |
| | | GACTGGATTCTGGAGAAAACTCAAGCAGTGTGA |
| | | ATGGGTTCCCAGGGGCCATTGGAGTCCCTGAAG |
| | | GACCCAGGATTTGCTGGGAGAGGGTGTTGAGTT |
| | | CACTGTGCCAGCATGCTTCCTCCACAGTAACAC |
| | | GCTGAAGGGGCTTGGTGTTTGTAAGAAAATGCT |
| | | AGAAGAAAACAAACTGTCACAAGTTGTTATGTC |
| | | CAAAACTCCCGTTCTATGATCGTTGTAGTTTGT |
| | | TTGAGCATTCAGTCTCTTTGTTTTTGATCACGC |
| | | TTCTATGGAGTCCAAGAATTACCATAAGGCAAT |
| | | ATTTCTGAAGATTACTATATAGGCAGATATAGC |
| | | AGAAAATAACCAAGTAGTGGCAGTGGGGATCAG |
| | | GCAGAAGAACTGGTAAAAGAAGCCACCATAAAT |
| | | AGATTTGTTCGATGAAAGATGAAAACTGGAAGA |
| | | AAGGAGAACAAAGACAGTCTTCACCATTTTGCA |
| | | GGAATCTACACTCTGCCTATGTGAACACATTTC |
| | | TTTTGTAAAGAAAGAAATTGATTGCATTTAATG |
| | | GCAGATTTTCAGAATAGTCAGGAATTCTTGTCA |
| | | TTTCCATTTTAAAATATATATTAAAAAAAATCA |
| | | GTTCGAGTAGACACGAGCTAAGAGTGAATGTGA |
| | | AGATAACAGAATTTCTGTGTGGAAGAGGATTAC |
| | | AAGCAGCAATTTACCTGGAAGTGATACCTTAGG |
| | | GGCAATCTTGAAGATACACTTTCCTGAAAAATG |
| | | ATTTGTGATGGATTGTATATTTATTTAAAATAT |
| | | CTTGGGAGGGGAGGCTGATGGAGATAGGGAGCA |
| | | TGCTCAAACCTCCCTAAGACAAGCTGCTGCTGT |
| | | GACTATGGGCTCCCAAAGAGCTAGATCGTATAT |
| | | TTATTTGACAAAAATCACCATAGACTGCATCCA |
| | | TACTACAGAGAAAAAACAATTAGGGCGCAAATG |
| | | GATAGTTACAGTAAAGTCTTCAGCAAGCAGCTG |
| | | CCTGTATTCTAAGCACTGGGATTTTCTGTTTCG |
| | | TGCAAATATTTATCTCATTATTGTTGTGATCTA |
| | | GTTCAATAACCTAGAATTTGAATTGTCACCACA |
| | | TAGCTTTCAATCTGTGCCAACAACTATACAATT |
| | | CATCAAGTGTG |
| Antibody 2 | | |
| HCDR1 (Kabat) | 3 | TAAMS |
| HCDR2 (Kabat) | 4 | GISGSGSSTYYADSVKG |
| HCDR3 (Kabat) | 5 | ELSYLYSGYYFDY |
| HCDR1 (Chothia) | 6 | GFTFSTA |
| HCDR2 (Chothia) | 7 | SGSGSS |
| HCDR3 (Chothia) | 8 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTFSTAAMS |
| HCDR2 (Combined) | 4 | GISGSGSSTYYADSVKG |
| HCDR3 (Combined) | 5 | ELSYLYSGYYFDY |
| VH | 9 | QVQLLESGGGLVQPGGSLRLSCAASGFTFSTAA |
| | | MSWVRQAPGKGLEWVSGISGSGSSTYYADSVKG |
| | | RFTISRDNSKNTLYLQMNSLRAEDTAVYYCARE |
| | | LSYLYSGYYFDYWGQGTLVTVSS |
| DNA encoding VH | 10 | CAGGTGCAATTGCTGGAAAGCGGCGGTGGCCTG |
| | | GTGCAGCCGGGTGGCAGCCTGCGTCTGAGCTGC |
| | | GCGGCGTCCGGATTCACCTTTTCTACTGCTGCT |
| | | ATGTCTTGGGTGCGCCAGGCCCCGGGCAAAGGT |
| | | CTCGAGTGGGTTTCCGGTATCTCTGGTTCTGGT |
| | | TCTTCTACCTACTATGCGGATAGCGTGAAAGGC |
| | | CGCTTTACCATCAGCCGCGATAATTCGAAAAAC |
| | | ACCCTGTATCTGCAAATGAACAGCCTGCGTGCG |
| | | GAAGATACGGCCGTGTATTATTGCGCGCGTGAA |
| | | CTGTCTTACCTGTACTCTGGTTACTACTTCGAT |
| | | TACTGGGGCCAAGGCACCCTGGTGACTGTTAGC |
| | | TCA |
| Heavy Chain | 11 | QVQLLESGGGLVQPGGSLRLSCAASGFTFSTAA |
| | | MSWVRQAPGKGLEWVSGISGSGSSTYYADSVKG |
| | | RFTISRDNSKNTLYLQMNSLRAEDTAVYYCARE |
| | | LSYLYSGYYFDYWGQGTLVTVSSASTKGPSVFP |
| | | LAPSSKSTSGGTAALGCLVKDYFPEPVTVSWNS |
| | | GALTSGVHTFPAVLQSSGLYSLSSWTVPSSSLG |
| | | TQTYICNVNHKPSNTKVDKRVEPKSCDKTHTCP |
| | | PCPAPEAAGGPSVFLFPPKPKDTLMISRTPEVT |
| | | CVVVDVSHEDPEVKFNWYVDGVEVHNAKTKPRE |
| | | EQYNSTYRVVSVLTVLHQDWLNGKEYKCKVSNK |
| | | ALPAPIEKTISKAKGQPREPQVYTLPPSREEMT |
| | | KNQVSLTCLVKGFYPSDIAVEWESNGQPENNYK |
| | | TTPPVLDSDGSFFLYSKLTVDKSRWQQGNVFSC |
| | | SVMHEALHNHYTQKSLSLSPGK |
| DNA encoding Heavy | 12 | CAGGTGCAATTGCTGGAAAGCGGCGGTGGCCTG |
| Chain | | GTGCAGCCGGGTGGCAGCCTGCGTCTGAGCTGC |
| | | GCGGCGTCCGGATTCACCTTTTCTACTGCTGCT |
| | | ATGTCTTGGGTGCGCCAGGCCCCGGGCAAAGGT |
| | | CTCGAGTGGGTTTCCGGTATCTCTGGTTCTGGT |
| | | TCTTCTACCTACTATGCGGATAGCGTGAAAGGC |
| | | CGCTTTACCATCAGCCGCGATAATTCGAAAAAC |
| | | ACCCTGTATCTGCAAATGAACAGCCTGCGTGCG |
| | | GAAGATACGGCCGTGTATTATTGCGCGCGTGAA |
| | | CTGTCTTACCTGTACTCTGGTTACTACTTCGAT |
| | | TACTGGGGCCAAGGCACCCTGGTGACTGTTAGC |
| | | TCAGCCTCCACCAAGGGTCCATCGGTCTTCCCC |
| | | CTGGCACCCTCCTCCAAGAGCACCTCTGGGGGC |
| | | ACAGCGGCCCTGGGCTGCCTGGTCAAGGACTAC |
| | | TTCCCCGAACCGGTGACGGTGTCGTGGAACTCA |
| | | GGCGCCCTGACCAGCGGCGTGCACACCTTCCCG |
| | | GCTGTCCTACAGTCCTCAGGACTCTACTCCCTC |
| | | AGCAGCGTGGTGACCGTGCCCTCCAGCAGCTTG |
| | | GGCACCCAGACCTACATCTGCAACGTGAATCAC |
| | | AAGCCCAGCAACACCAAGGTGGACAAGAGAGTT |
| | | GAGCCCAAATCTTGTGACAAAACTCACACATGC |
| | | CCACCGTGCCCAGCACCTGAAGCAGCGGGGGGA |
| | | CCGTCAGTCTTCCTCTTCCCCCCAAAACCCAAG |
| | | GACACCCTCATGATCTCCCGGACCCCTGAGGTC |
| | | ACATGCGTGGTGGTGGACGTGAGCCACGAAGAC |
| | | CCTGAGGTCAAGTTCAACTGGTACGTGGACGGC |
| | | GTGGAGGTGCATAATGCCAAGACAAAGCCGCGG |
| | | GAGGAGCAGTACAACAGCACGTACCGGGTGGTC |
| | | AGCGTCCTCACCGTCCTGCACCAGGACTGGCTG |
| | | AATGGCAAGGAGTACAAGTGCAAGGTCTCCAAC |
| | | AAAGCCCTCCCAGCCCCCATCGAGAAAACCATC |
| | | TCCAAAGCCAAAGGGCAGCCCCGAGAACCACAG |
| | | GTGTACACCCTGCCCCCATCCCGGGAGGAGATG |
| | | ACCAAGAACCAGGTCAGCCTGACCTGCCTGGTC |
| | | AAAGGCTTCTATCCCAGCGACATCGCCGTGGAG |
| | | TGGGAGAGCAATGGGCAGCCGGAGAACAACTAC |
| | | AAGACCACGCCTCCCGTGCTGGACTCCGACGGC |
| | | TCCTTCTTCCTCTACAGCAAGCTCACCGTGGAC |
| | | AAGAGCAGGTGGCAGCAGGGGAACGTCTTCTCA |
| | | TGCTCCGTGATGCATGAGGCTCTGCACAACCAC |
| | | TACACGCAGAAGAGCCTCTCCCTGTCTCCGGGT |
| | | AAA |
| LCDR1 (Kabat) | 13 | SGSSSNIGSNDVS |
| LCDR2 (Kabat) | 14 | KNYNRPS |
| LCDR3 (Kabat) | 15 | SAWDQRQFDVV |
| LCDR1 (Chothia) | 16 | SSSNIGSND |
| LCDR2 (Chothia) | 17 | KNY |
| LCDR3 (Chothia) | 18 | WDQRQFDV |
| LCDR1 (IMGT) | 47 | SSNIGSND |
| LCDR2 (IMGT) | 37 | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 19 | DIVLTQPPSVSGAPGQRVTISCSGSSSNIGSND |
| | | VSWYQQLPGTAPKLLIYKNYNRPSGVPDRFSGS |
| | | KSGTSASLAITGLQAEDEADYYCSAWDQRQFDV |
| | | VFGGGTKLTVL |
| DNA encoding VL | 20 | GATATCGTGCTGACCCAGCCGCCGAGCGTGAGC |
| | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTT |
| | | GTGTTTGGCGGCGGCACGAAGTTAACCGTCCTA |
| Light Chain | 21 | DIVLTQPPSVSGAPGQRVTISCSGSSSNIGSND |
| | | VSWYQQLPGTAPKLLIYKNYNRPSGVPDRFSGS |
| | | KSGTSASLAITGLQAEDEADYYCSAWDQRQFDV |
| | | VFGGGTKLTVLGQPKAAPSVTLFPPSSEELQAN |
| | | KATLVCLISDFYPGAVTVAWKADSSPVKAGVET |
| | | TTPSKQSNNKYAASSYLSLTPEQWKSHRSYSCQ |
| | | VTHEGSTVEKTVAPTECS |
| DNA encoding Light | 22 | GATATCGTGCTGACCCAGCCGCCGAGCGTGAGC |
| Chain | | GGTGCACCGGGCCAGCGCGTGACCATTAGCTGT |
| | | AGCGGCAGCAGCAGCAACATTGGTTCTAACGAC |
| | | GTGTCTTGGTACCAGCAGCTGCCGGGCACGGCG |
| | | CCGAAACTGCTGATCTACAAAAACTACAACCGC |
| | | CCGAGCGGCGTGCCGGATCGCTTTAGCGGATCC |
| | | AAAAGCGGCACCAGCGCCAGCCTGGCGATTACC |
| | | GGCCTGCAAGCAGAAGACGAAGCGGATTATTAC |
| | | TGCTCTGCTTGGGACCAGCGTCAGTTCGACGTT |
| | | GTGTTTGGCGGCGGCACGAAGTTAACCGTCCTA |
| | | GGTCAGCCCAAGGCTGCCCCCTCGGTCACTCTG |
| | | TTCCCGCCCTCCTCTGAGGAGCTTCAAGCCAAC |
| | | AAGGCCACACTGGTGTGTCTCATAAGTGACTTC |
| | | TACCCGGGAGCCGTGACAGTGGCCTGGAAGGCA |
| | | GATAGCAGCCCCGTCAAGGCGGGAGTGGAGACC |
| | | ACCACACCCTCCAAACAAAGCAACAACAAGTAC |
| | | GCGGCCAGCAGCTATCTGAGCCTGACGCCTGAG |
| | | CAGTGGAAGTCCCACAGAAGCTACAGCTGCCAG |
| | | GTCACGCATGAAGGGAGCACCGTGGAGAAGACA |
| | | GTGGCCCCTACAGAATGTTCA |
| Antibody 1 | | |
| HCDR1 (Kabat) | 23 | TAAMS |
| HCDR2 (Kabat) | 24 | GISGSGSSTYYADSVKG |
| HCDR3 (Kabat) | 25 | ELSYLYSGYYFDY |
| HCDR1 (Chothia) | 26 | GFTFSTA |
| HCDR2 (Chothia) | 27 | SGSGSS |
| HCDR3 (Chothia) | 28 | ELSYLYSGYYFDY |
| HCDR1 (IMGT) | 43 | GFTFSTAA |
| HCDR2 (IMGT) | 44 | ISGSGSST |
| HCDR3 (IMGT) | 45 | ARELSYLYSGYYFDY |
| HCDR1 (Combined) | 46 | GFTFSTAAMS |
| HCDR2 (Combined) | 4 | GISGSGSSTYYADSVKG |
| HCDR3 (Combined) | 5 | ELSYLYSGYYFDY |
| VH | 29 | QVQLLESGGGLVQPGGSLRLSCAASGFTFSTAA |
| | | MSWVRQAPGKGLEWVSGISGSGSSTYYADSVKG |
| | | RFTISRDNSKNTLYLQMNSLRAEDTAVYYCARE |
| | | LSYLYSGYYFDYWGQGTLVTVSS |
| DNA encoding VH | 30 | CAGGTGCAGCTGCTGGAATCAGGCGGCGGACTG |
| | | GTGCAGCCTGGCGGTAGCCTGAGACTGAGCTGC |
| | | GCTGCTAGTGGCTTCACCTTTAGCACCGCCGCT |
| | | ATGAGCTGGGTTCGACAGGCCCCAGGGAAAGGC |
| | | CTCGAGTGGGTCTCAGGGATTAGCGGTAGCGGC |
| | | TCTAGCACCTACTACGCCGATAGCGTGAAGGGC |
| | | CGGTTCACTATCTCTAGGGATAACTCTAAGAAC |
| | | ACCCTGTACCTGCAGATGAATAGCCTGAGAGCC |
| | | GAGGACACCGCCGTCTACTACTGCGCTAGAGAG |
| | | CTGAGCTACCTGTATAGCGGCTACTACTTCGAC |
| | | TACTGGGGTCAAGGCACCCTGGTCACCGTGTCT |
| | | AGC |
| Heavy Chain | 31 | QVQLLESGGGLVQPGGSLRLSCAASGFTFSTAA |
| | | MSWVRQAPGKGLEWVSGISGSGSSTYYADSVKG |
| | | RFTISRDNSKNTLYLQMNSLRAEDTAVYYCARE |
| | | LSYLYSGYYFDYWGQGTLVTVSSASTKGPSVFP |
| | | LAPSSKSTSGGTAALGCLVKDYFPEPVTVSWNS |
| | | GALTSGVHTFPAVLQSSGLYSLSSWTVPSSSLG |
| | | TQTYICNVNHKPSNTKVDKRVEPKSCDKTHTCP |
| | | PCPAPELLGGPSVFLFPPKPKDTLMISRTPEVT |
| | | CVVVAVSHEDPEVKFNWYVDGVEVHNAKTKPRE |
| | | EQYNSTYRVVSVLTVLHQDWLNGKEYKCKVSNK |
| | | ALAAPIEKTISKAKGQPREPQVYTLPPSREEMT |
| | | KNQVSLTCLVKGFYPSDIAVEWESNGQPENNYK |
| | | TTPPVLDSDGSFFLYSKLTVDKSRWQQGNVFSC |
| | | SVMHEALHNHYTQKSLSLSPGK |
| DNA encoding Heavy | 32 | CAGGTGCAGCTGCTGGAATCAGGCGGCGGACTG |
| Chain | | GTGCAGCCTGGCGGTAGCCTGAGACTGAGCTGC |
| | | GCTGCTAGTGGCTTCACCTTTAGCACCGCCGCT |
| | | ATGAGCTGGGTTCGACAGGCCCCAGGGAAAGGC |
| | | CTCGAGTGGGTCTCAGGGATTAGCGGTAGCGGC |
| | | TCTAGCACCTACTACGCCGATAGCGTGAAGGGC |
| | | CGGTTCACTATCTCTAGGGATAACTCTAAGAAC |
| | | ACCCTGTACCTGCAGATGAATAGCCTGAGAGCC |
| | | GAGGACACCGCCGTCTACTACTGCGCTAGAGAG |
| | | CTGAGCTACCTGTATAGCGGCTACTACTTCGAC |
| | | TACTGGGGTCAAGGCACCCTGGTCACCGTGTCT |
| | | AGCGCTAGCACTAAGGGCCCCTCCGTGTTCCCT |
| | | CTGGCCCCTTCCAGCAAGTCTACCTCCGGCGGC |
| | | ACAGCTGCTCTGGGCTGCCTGGTCAAGGACTAC |
| | | TTCCCTGAGCCTGTGACAGTGTCCTGGAACTCT |
| | | GGCGCCCTGACCTCTGGCGTGCACACCTTCCCT |
| | | GCCGTGCTGCAGTCCTCCGGCCTGTACTCCCTG |
| | | TCCTCCGTGGTCACAGTGCCTTCAAGCAGCCTG |
| | | GGCACCCAGACCTATATCTGCAACGTGAACCAC |
| | | AAGCCTTCCAACACCAAGGTGGACAAGCGGGTG |
| | | GAGCCTAAGTCCTGCGACAAGACCCACACCTGT |
| | | CCTCCCTGCCCTGCTCCTGAACTGCTGGGCGGC |
| | | CCTTCTGTGTTCCTGTTCCCTCCAAAGCCCAAG |
| | | GACACCCTGATGATCTCCCGGACCCCTGAAGTG |
| | | ACCTGCGTGGTGGTGGCCGTGTCCCACGAGGAT |
| | | CCTGAAGTGAAGTTCAATTGGTACGTGGACGGC |
| | | GTGGAGGTGCACAACGCCAAGACCAAGCCTCGG |
| | | GAGGAACAGTACAACTCCACCTACCGGGTGGTG |
| | | TCCGTGCTGACCGTGCTGCACCAGGACTGGCTG |
| | | AACGGCAAAGAGTACAAGTGCAAAGTCTCCAAC |
| | | AAGGCCCTGGCCGCCCCTATCGAAAAGACAATC |
| | | TCCAAGGCCAAGGGCCAGCCTAGGGAACCCCAG |
| | | GTGTACACCCTGCCACCCAGCCGGGAGGAAATG |
| | | ACCAAGAACCAGGTGTCCCTGACCTGTCTGGTC |
| | | AAGGGCTTCTACCCTTCCGATATCGCCGTGGAG |
| | | TGGGAGTCTAACGGCCAGCCTGAGAACAACTAC |
| | | AAGACCACCCCTCCTGTGCTGGACTCCGACGGC |
| | | TCCTTCTTCCTGTACTCCAAACTGACCGTGGAC |
| | | AAGTCCCGGTGGCAGCAGGGCAACGTGTTCTCC |
| | | TGCTCCGTGATGCACGAGGCCCTGCACAACCAC |
| | | TACACCCAGAAGTCCCTGTCCCTGTCTCCCGGC |
| | | AAG |
| LCDR1 (Kabat) | 33 | SGSSSNIGSNDVS |
| LCDR2 (Kabat) | 34 | KNYNRPS |
| LCDR3 (Kabat) | 35 | SAWDQRQFDVV |
| LCDR1 (Chothia) | 36 | SSSNIGSND |
| LCDR2 (Chothia) | 37 | KNY |
| LCDR3 (Chothia) | 38 | WDQRQFDV |
| LCDR1 (IMGT) | 47 | SSNIGSND |
| LCDR2 (IMGT) | 37 | KNY |
| LCDR3 (IMGT) | 15 | SAWDQRQFDVV |
| LCDR1 (Combined) | 33 | SGSSSNIGSNDVS |
| LCDR2 (Combined) | 14 | KNYNRPS |
| LCDR3 (Combined) | 15 | SAWDQRQFDVV |
| VL | 39 | QSVLTQPPSASGTPGQRVTISCSGSSSNIGSND |
| | | VSWYQQLPGTAPKLLIYKNYNRPSGVPDRFSGS |
| | | KSGTSASLAISGLQSEDEADYYCSAWDQRQFDV |
| | | VFGGGTKLTVL |
| DNA encoding VL | 40 | CAGTCAGTCCTGACTCAGCCCCCTAGCGCTAGT |
| | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| Light Chain | 41 | QSVLTQPPSASGTPGQRVTISCSGSSSNIGSND |
| | | VSWYQQLPGTAPKLLIYKNYNRPSGVPDRFSGS |
| | | KSGTSASLAISGLQSEDEADYYCSAWDQRQFDV |
| | | VFGGGTKLTVLGQPKAAPSVTLFPPSSEELQAN |
| | | KATLVCLISDFYPGAVTVAWKADSSPVKAGVET |
| | | TTPSKQSNNKYAASSYLSLTPEQWKSHRSYSCQ |
| | | VTHEGSTVEKTVAPTECS |
| DNA encoding Light | 42 | CAGTCAGTCCTGACTCAGCCCCCTAGCGCTAGT |
| Chain | | GGCACCCCTGGTCAAAGAGTGACTATTAGCTGT |
| | | AGCGGCTCTAGCTCTAATATCGGCTCTAACGAC |
| | | GTCAGCTGGTATCAGCAGCTGCCCGGCACCGCC |
| | | CCTAAGCTGCTGATCTATAAGAACTATAATAGG |
| | | CCTAGCGGCGTGCCCGATAGGTTTAGCGGATCT |
| | | AAATCAGGGACTTCTGCTAGTCTGGCTATTAGC |
| | | GGCCTGCAGTCAGAGGACGAGGCCGACTACTAC |
| | | TGTAGCGCCTGGGATCAGCGTCAGTTCGACGTG |
| | | GTGTTCGGCGGAGGCACTAAGCTGACCGTGCTG |
| | | GGTCAACCTAAGGCTGCCCCCAGCGTGACCCTG |
| | | TTCCCCCCCAGCAGCGAGGAGCTGCAGGCCAAC |
| | | AAGGCCACCCTGGTGTGCCTGATCAGCGACTTC |
| | | TACCCAGGCGCCGTGACCGTGGCCTGGAAGGCC |
| | | GACAGCAGCCCCGTGAAGGCCGGCGTGGAGACC |
| | | ACCACCCCCAGCAAGCAGAGCAACAACAAGTAC |
| | | GCCGCCAGCAGCTACCTGAGCCTGACCCCCGAG |
| | | CAGTGGAAGAGCCACAGGTCCTACAGCTGCCAG |
| | | GTGACCCACGAGGGCAGCACCGTGGAAAAGAC |
| | | CGTGGCCCCAACCGAGTGCAGC |

In some embodiments, other antibodies for use in the methods or formulations described herein (e.g., methods for treating a subject afflicted with or at risk of developing a thromboembolic disorder) include those where the amino acids or nucleic acids encoding the amino acids have been mutated, yet have at least 60, 65, 70, 75, 80, 85, 90, or 95 percent identity to the sequences described in Table 1. Some embodiments include mutant amino acid sequences wherein no more than 1, 2, 3, 4 or 5 amino acids have been mutated in the variable regions when compared with the variable regions depicted in the sequence described in Table 1, while retaining substantially the same antigen binding activity.

Since each of these antibodies can bind to FXI and/or FXIa, the VH, VL, full length light chain, and full length heavy chain sequences (amino acid sequences and the nucleotide sequences encoding the amino acid sequences) can be “mixed and matched” to create other FXI and/or FXIa-binding antibodies of the present disclosure. Such “mixed and matched” FXI and/or FXIa-binding antibodies can be tested using the binding assays known in the art (e.g., ELISAs, and other assays described in the Example section). When these chains are mixed and matched, a VH sequence from a particular VH/VL pairing should be replaced with a structurally similar VH sequence. Likewise a full length heavy chain sequence from a particular full length heavy chain/full length light chain pairing should be replaced with a structurally similar full length heavy chain sequence. Likewise, a VL sequence from a particular VH/VL pairing should be replaced with a structurally similar VL sequence. Likewise a full length light chain sequence from a particular full length heavy chain/full length light chain pairing should be replaced with a structurally similar full length light chain sequence.

Accordingly, in one aspect, for use in the methods described herein (e.g., methods for treating a subject afflicted with or at risk of developing a thromboembolic disorder), the present disclosure provides an isolated antibody or antigen binding region thereof having: a heavy chain variable domain comprising an amino acid sequence selected from the group consisting of SEQ ID NOs: 9 and 29, and a light chain variable domain comprising an amino acid sequence selected from the group consisting of SEQ ID NOs: 19 and 39, wherein the antibody specifically binds to FXI and/or FXIa (e.g., human, rabbit, cynomolgus monkey, and baboon FXIa). In another aspect, for use in the formulations described herein (e.g., the formulation in the vial, the intravenous drug delivery formulation), the present disclosure provides an isolated antibody or antigen binding region thereof having: a heavy chain variable domain comprising an amino acid sequence selected from the group consisting of SEQ ID NOs: 9 and 29, and a light chain variable domain comprising an amino acid sequence selected from the group consisting of SEQ ID NOs: 19 and 39, wherein the antibody specifically binds to FXI and/or FXIa (e.g., human, rabbit, cynomolgus monkey, and baboon FXIa).

More specifically, in certain aspects, the present disclosure provides an isolated antibody or antigen binding fragment thereof having a heavy chain variable domain and a light chain variable domain comprising amino acid sequences selected from SEQ ID NOs: 9 and 29; or 19 and 39, respectively.

In a specific embodiment for use in the methods described herein (e.g., methods for treating a subject afflicted with or at risk of developing a thromboembolic disorder), an antibody or antigen binding fragment thereof provided herein which specifically binds to human FXI and/or FXIa, comprises a heavy chain variable region comprising the amino acid sequence of SEQ ID NO: 9, and a light chain variable region comprising the amino acid sequence of SEQ ID NO: 19. In a specific embodiment for use in the formulations described herein (e.g., the formulation in the vial, the intravenous drug delivery formulation), an antibody or antigen binding fragment thereof provided herein which specifically binds to human FXI and/or FXIa, comprises a heavy chain variable region comprising the amino acid sequence of SEQ ID NO: 9, and a light chain variable region comprising the amino acid sequence of SEQ ID NO: 19.

In a specific embodiment for use in the methods described herein (e.g., methods for treating a subject afflicted with or at risk of developing a thromboembolic disorder), an antibody or antigen binding fragment thereof provided herein which specifically binds to human FXI and/or FXIa, comprises a heavy chain variable region comprising the amino acid sequence of SEQ ID NO: 29, and a light chain variable region comprising the amino acid sequence of SEQ ID NO: 39. In a specific embodiment for use in the formulations described herein (e.g., the formulation in the vial, the intravenous drug delivery formulation), an antibody or antigen binding fragment thereof provided herein which specifically binds to human FXI and/or FXIa, comprises a heavy chain variable region comprising the amino acid sequence of SEQ ID NO: 29, and a light chain variable region comprising the amino acid sequence of SEQ ID NO: 39.

In another aspect for use in the methods described herein, the present disclosure provides (i) an isolated antibody having: a full length heavy chain comprising an amino acid sequence that has been optimized for expression in a mammalian cell selected from the group consisting of SEQ ID NOs: 11 or 31, and a full length light chain comprising an amino acid sequence that has been optimized for expression in a mammalian cell selected from the group consisting of SEQ ID NOs: 21 or 41; or (ii) a functional protein comprising an antigen binding portion thereof. More specifically, in certain aspects, the present disclosure provides an isolated antibody or antigen binding region thereof having a heavy chain and a light chain comprising amino acid sequences selected from SEQ ID NOs: 11 and 31; or 21 and 41, respectively. In another aspect for use in the formulations described herein, the present disclosure provides (i) an isolated antibody having: a full length heavy chain comprising an amino acid sequence that has been optimized for expression in a mammalian cell selected from the group consisting of SEQ ID NOs: 11 or 31, and a full length light chain comprising an amino acid sequence that has been optimized for expression in a mammalian cell selected from the group consisting of SEQ ID NOs: 21 or 41; or (ii) a functional protein comprising an antigen binding portion thereof. More specifically, in certain aspects, the present disclosure provides an isolated antibody or antigen binding region thereof having a heavy chain and a light chain comprising amino acid sequences selected from SEQ ID NOs: 11 and 31; or 21 and 41, respectively.

In a specific embodiment for use in the methods described herein, an antibody or antigen binding fragment thereof provided herein which specifically binds to human FXI and/or FXIa, comprises a heavy chain comprising the amino acid sequence of SEQ ID NO: 11, and a light chain comprising the amino acid sequence of SEQ ID NO: 21. In a specific embodiment for use in the formulations described herein, an antibody or antigen binding fragment thereof provided herein which specifically binds to human FXI and/or FXIa, comprises a heavy chain comprising the amino acid sequence of SEQ ID NO: 11, and a light chain comprising the amino acid sequence of SEQ ID NO: 21.

In a specific embodiment for use in the methods described herein, an antibody or antigen binding fragment thereof provided herein which specifically binds to human FXI and/or FXIa, comprises a heavy chain variable region comprising the amino acid sequence of SEQ ID NO: 31, and a light chain variable region comprising the amino acid sequence of SEQ ID NO: 41. In a specific embodiment for use in the formulations described herein, an antibody or antigen binding fragment thereof provided herein which specifically binds to human FXI and/or FXIa, comprises a heavy chain variable region comprising the amino acid sequence of SEQ ID NO: 31, and a light chain variable region comprising the amino acid sequence of SEQ ID NO: 41.

The terms “complementarity determining region,” and “CDR,” as used herein refer to the sequences of amino acids within antibody variable regions which confer antigen specificity and binding affinity. In general, there are three CDRs in each heavy chain variable region (HCDR1, HCDR2, HCDR3) and three CDRs in each light chain variable region (LCDR1, LCDR2, LCDR3).

The precise amino acid sequence boundaries of a given CDR can be readily determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), Lefranc et al., (2003) Dev. Comp. Immunol., 27, 55-77 (“IMGT” numbering scheme), or the “Combined” system.

For example, under Kabat, the CDR amino acid residues of antibody Antibody 2 in the heavy chain variable domain (VH) are numbered 31-35 (HCDR1), 50-66 (HCDR2), and 99-111 (HCDR3); and the CDR amino acid residues in the light chain variable domain (VL) are numbered 22-35 (LCDR1), 51-57 (LCDR2), and 90-100 (LCDR3). Under Chothia the CDR amino acids in the VH are numbered 26-32 (HCDR1), 52-57 (HCDR2), and 99-111 (HCDR3); and the amino acid residues in VL are numbered 25-33 (LCDR1), 51-53 (LCDR2), and 92-99 (LCDR3). By combining the CDR definitions of both Kabat and Chothia, the CDRs consist of amino acid residues 26-35 (HCDR1), 50-66 (HCDR2), and 99-111 (HCDR3) in human VH and amino acid residues 22-35 (LCDR1), 51-57 (LCDR2), and 90-100 (LCDR3) in human VL. By combining the CDR definitions of both Kabat and Chothia, the “Combined” CDRs consist of amino acid residues 26-35 (HCDR1), 50-66 (HCDR2), and 99-108 (HCDR3) in human VH and amino acid residues 24-38 (LCDR1), 54-60 (LCDR2), and 93-101 (LCDR3) in human VL. As another example, under IMGT, the CDR amino acid residues in the heavy chain variable domain (VH) are numbered 26-33 (HCDR1), 51-58 (HCDR2), and 97-108 (HCDR3); and the CDR amino acid residues in the light chain variable domain (VL) are numbered 27-36 (LCDR1), 54-56 (LCDR2), and 93-101 (LCDR3). Table 1 provides exemplary Kabat, Chothia, Combined, and IMGT HCDR1, HCDR2, HCDR3, LCDR1, LCDR2, and LCDR3 for anti-FXI/FXIa antibodies, e.g., Antibody 2 and Antibody 1. In another aspect, the present disclosure provides FXIa binding antibodies that comprise the heavy chain and light chain CDR1s, CDR2s, and CDR3s as described in Table 1, or combinations thereof. The amino acid sequences of the VH CDR1s of the antibodies are shown in SEQ ID NOs: 3 and 23. The amino acid sequences of the VH CDR2s of the antibodies are shown in SEQ ID NOs: 4 and 24. The amino acid sequences of the VH CDR3s of the antibodies are shown in SEQ ID NOs: 5 and 25. The amino acid sequences of the VL CDR1s of the antibodies are shown in SEQ ID NOs: 13 and 33. The amino acid sequences of the VL CDR2s of the antibodies are shown in SEQ ID NOs: 14 and 34. The amino acid sequences of the VL CDR3s of the antibodies are shown in SEQ ID NOs: 15 and 35. These CDR regions are delineated using the Kabat system.

Alternatively, as defined using the Chothia system (Al-Lazikani et al., (1997) JMB 273,927-948), the amino acid sequences of the VH CDR1s of the antibodies are shown in SEQ ID NOs: 6 and 26. The amino acid sequences of the VH CDR2s of the antibodies and are shown in SEQ ID NOs: 7 and 27. The amino acid sequences of the VH CDR3s of the antibodies are shown in SEQ ID NOs: 8 and 28. The amino acid sequences of the VL CDR1s of the antibodies are shown in SEQ ID NOs: 16 and 36. The amino acid sequences of the VL CDR2s of the antibodies are shown in SEQ ID NOs: 17 and 37. The amino acid sequences of the VL CDR3s of the antibodies are shown in SEQ ID NOs: 18 and 38.

Alternatively, as defined using the Combined system, the amino acid sequences of the VH CDR1 of the antibodies are shown in SEQ ID NO: 46. The amino acid sequences of the VH CDR2 of the antibodies and are shown in SEQ ID NO: 4. The amino acid sequences of the VH CDR3 of the antibodies are shown in SEQ ID NO: 5. The amino acid sequences of the VL CDR1 of the antibodies are shown in SEQ ID NO: 33. The amino acid sequences of the VL CDR2 of the antibodies are shown in SEQ ID NO: 14. The amino acid sequences of the VL CDR3 of the antibodies are shown in SEQ ID NO: 15.

Alternatively, as defined using the IMGT numbering scheme, the amino acid sequences of the VH CDR1 of the antibodies are shown in SEQ ID NO: 43. The amino acid sequences of the VH CDR2 of the antibodies and are shown in SEQ ID NO: 44. The amino acid sequences of the VH CDR3 of the antibodies are shown in SEQ ID NO: 45. The amino acid sequences of the VL CDR1 of the antibodies are shown in SEQ ID NO: 47. The amino acid sequences of the VL CDR2 of the antibodies are shown in SEQ ID NO: 37. The amino acid sequences of the VL CDR3 of the antibodies are shown in SEQ ID NO: 15.

Given that each of these antibodies can bind to FXI and/or FXIa and that antigen-binding specificity is provided primarily by the CDR1, 2 and 3 regions, the VH CDR1, 2 and 3 sequences and VL CDR1, 2 and 3 sequences can be “mixed and matched” (e.g., CDRs from different antibodies can be mixed and matched, although each antibody preferably contains a VH CDR1, 2 and 3 and a VL CDR1, 2 and 3 to create other FXI and/or FXIa binding molecules of the present disclosure). Such “mixed and matched” FXI and/or FXIa binding antibodies can be tested using the binding assays known in the art and those described in the Examples (e.g., ELISAs, SET, BIACORE′ assays). When VH CDR sequences are mixed and matched, the CDR1, CDR2 and/or CDR3 sequence from a particular VH sequence should be replaced with a structurally similar CDR sequence(s). Likewise, when VL CDR sequences are mixed and matched, the CDR1, CDR2 and/or CDR3 sequence from a particular VL sequence should be replaced with a structurally similar CDR sequence(s). It will be readily apparent to the ordinarily skilled artisan that novel VH and VL sequences can be created by substituting one or more VH and/or VL CDR region sequences with structurally similar sequences from the CDR sequences shown herein for monoclonal antibodies of the present disclosure. In addition to the foregoing, in one embodiment, the antigen binding fragments of the antibodies described herein can comprise a VH CDR1, 2, and 3, or a VL CDR 1, 2, and 3, wherein the fragment binds to FXI and/or FXIa as a single variable domain. It is noted that the CDR sequences of Antibody 1 and Antibody 2 are identical.

In certain embodiments of the present disclosure, the antibodies or antigen binding fragments thereof may have the heavy and light chain sequences of the Fabs described in Table 1. More specifically, the antibody or antigen binding fragments thereof may have the heavy and light sequence of Antibody 2 and Antibody 1.

In other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Kabat and described in Table 1. In still other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by Chothia and described in Table 1. In other embodiments, the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by the Combined system and described in Table 1. In still other embodiments of the present disclosure the antibody or antigen binding fragment in that specifically binds FXI and/or FXIa comprises a heavy chain variable region CDR1, a heavy chain variable region CDR2, a heavy chain variable region CDR3, a light chain variable region CDR1, a light chain variable region CDR2, and a light chain variable region CDR3 as defined by IMGT and described in Table 1.

In a specific embodiment for use in the methods described herein, the present disclosure includes an antibody that specifically binds to FXI and/or FXIa comprising a heavy chain variable region CDR1 of SEQ ID NO: 3; a heavy chain variable region CDR2 of SEQ ID NO: 4; a heavy chain variable region CDR3 of SEQ ID NO: 5; a light chain variable region CDR1 of SEQ ID NO: 13; a light chain variable region CDR2 of SEQ ID NO: 14; and a light chain variable region CDR3 of SEQ ID NO: 15.

In a specific embodiment, the present disclosure includes an antibody that specifically binds to FXI and/or FXIa comprising a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35.

In a specific embodiment, the present disclosure includes an antibody that specifically binds to FXI and/or FXIa comprising a heavy chain variable region CDR1 of SEQ ID NO: 6; a heavy chain variable region CDR2 of SEQ ID NO: 7; a heavy chain variable region CDR3 of SEQ ID NO: 8; a light chain variable region CDR1 of SEQ ID NO: 16; a light chain variable region CDR2 of SEQ ID NO: 17; and a light chain variable region CDR3 of SEQ ID NO: 18.

In a specific embodiment, the present disclosure includes an antibody that specifically binds to FXI and/or FXIa comprising a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37; and a light chain variable region CDR3 of SEQ ID NO: 38.

In a specific embodiment, provided herein is an antibody that specifically binds to FXI and/or FXIa comprising a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37 and a light chain variable region CDR3 of SEQ ID NO: 15.

In a specific embodiment, provided herein is an antibody that specifically binds to FXI and/or FXIa comprising a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14 and a light chain variable region CDR3 of SEQ ID NO: 15.

In certain embodiments, the present disclosure includes antibodies or antigen binding fragments that specifically bind to FXI and/or FXIa as described in Table 1. In a specific embodiment for use in the methods described herein, the antibody, or antigen binding fragment, that binds FXI and/or FXIa is Antibody 2 and Antibody 1.

As used herein, a human antibody comprises heavy or light chain variable regions or full length heavy or light chains that are “the product of” or “derived from” a particular germline sequence if the variable regions or full length chains of the antibody are obtained from a system that uses human germline immunoglobulin genes. Such systems include immunizing a transgenic mouse carrying human immunoglobulin genes with the antigen of interest or screening a human immunoglobulin gene library displayed on phage with the antigen of interest. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody.

A human antibody that is “the product of” or “derived from” a particular human germline immunoglobulin sequence may contain amino acid differences as compared to the germline sequence, due to, for example, naturally occurring somatic mutations or intentional introduction of site-directed mutations. However, in the VH or VL framework regions, a selected human antibody typically is at least 90% identical in amino acids sequence to an amino acid sequence encoded by a human germline immunoglobulin gene and contains amino acid residues that identify the human antibody as being human when compared to the germline immunoglobulin amino acid sequences of other species (e.g., murine germline sequences). In certain cases, a human antibody may be at least 60%, 70%, 80%, 90%, or at least 95%, or even at least 96%, 97%, 98%, or 99% identical in amino acid sequence to the amino acid sequence encoded by the germline immunoglobulin gene.

Typically, a recombinant human antibody will display no more than 10 amino acid differences from the amino acid sequence encoded by the human germline immunoglobulin gene in the VH or VL framework regions. In certain cases, the human antibody may display no more than 5, or even no more than 4, 3, 2, or 1 amino acid difference from the amino acid sequence encoded by the germline immunoglobulin gene. Examples of human germline immunoglobulin genes include, but are not limited to the variable domain germline fragments described below, as well as DP47 and DPK9.

### Homologous Antibodies

In yet other embodiments for use in the methods described herein (e.g., methods for treating a subject afflicted with or at risk of developing a thromboembolic disorder), the present disclosure provides an antibody, or an antigen binding fragment thereof, comprising amino acid sequences that are homologous to the sequences described in Table 1 (e.g., SEQ ID NOs: 29, 31, 39, or 41), and the antibody binds to a FXI and/or FXIa protein (e.g., human, rabbit, cynomolgus monkey, and baboon FXIa), and retains the desired functional properties of those antibodies described in Table 1 such as Antibody 2 and Antibody 1. In specific aspects, such homologous antibodies retain the CDR amino acid sequences described in Table 1 (e.g., Kabat CDRs, Chothia CDRs, IMGT CDRs, or Combined CDRs).

For example, in some embodiments the present disclosure provides an isolated antibody, or a functional antigen binding fragment thereof, comprising a heavy chain variable domain and a light chain variable domain, wherein the heavy chain variable domain comprises an amino acid sequence that is at least 80%, at least 90%, or at least 95% identical to an amino acid sequence selected from the group consisting of SEQ ID NOs: 9 and 29; the light chain variable domain comprises an amino acid sequence that is at least 80%, at least 90%, or at least 95% identical to an amino acid sequence selected from the group consisting of SEQ ID NOs: 19 and 39; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 9; the light chain variable domain comprises an amino acid sequence that is at least 80%, at least 90%, or at least 95% identical to the amino acid sequence of SEQ ID NO: 19; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain variable domain and a light chain variable domain, wherein the heavy chain variable domain comprises an amino acid sequence that is at least 80%, at least 90%, or at least 95% identical to the amino acid sequence of SEQ ID NO: 29; the light chain variable domain comprises an amino acid sequence that is at least 80%, at least 90%, or at least 95% identical to the amino acid sequence of SEQ ID NO: 39; and the antibody specifically binds to FXI and/or FXIa (e.g., human, rabbit, cynomolgus monkey, and baboon FXIa). In certain aspects of the present disclosure the heavy and light chain sequences further comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain other aspects of the present disclosure the heavy and light chain sequences further comprise HCDR1, HCDR2, HCDR3, LCDR1, LCDR2, and LCDR3 sequences as defined by Chothia, for example SEQ ID NOs: 6, 7, 8, 16, 17, and 18, respectively. In certain other aspects, the heavy and light chain sequences further comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain other aspects, the heavy and light chain sequences further comprise HCDR1, HCDR2, HCDR3, LCDR1, LCDR2, and LCDR3 sequences as defined by IMGT, for example SEQ ID NOs: 43, 44, 45, 47, 37, and 15, respectively.

In other embodiments for use in the methods described herein, the VH and/or VL amino acid sequences may be 50%, 60%, 70%, 80%, 90%, 95%, 96%, 97%, 98% or 99% identical to the sequences set forth in Table 1. In other embodiments for use in the formulations described herein, the VH and/or VL amino acid sequences may be 50%, 60%, 70%, 80%, 90%, 95%, 96%, 97%, 98% or 99% identical to the sequences set forth in Table 1. In other embodiments, the VH and/or VL amino acid sequences may be identical except for an amino acid substitution in no more than 1, 2, 3, 4 or 5 amino acid positions. An antibody having VH and VL regions having high (i.e., 80% or greater) identity to the VH and VL regions of those described in Table 1 can be obtained by mutagenesis (e.g., site-directed or PCR-mediated mutagenesis) of nucleic acid molecules encoding SEQ ID NOs: 10 or 30 and SEQ ID NOs: 20 and 40, respectively, followed by testing of the encoded altered antibody for retained function using the functional assays described herein.

In other embodiments for use in the methods described herein, the full length heavy chain and/or full length light chain amino acid sequences may be 50% 60%, 70%, 80%, 90%, 95%, 96%, 97%, 98% or 99% identical to the sequences set forth in Table 1 (e.g., SEQ ID NOs: 11 and/or 21, or 31 and/or 41). In other embodiments for use in the formulations described herein, the full length heavy chain and/or full length light chain amino acid sequences may be 50% 60%, 70%, 80%, 90%, 95%, 96%, 97%, 98% or 99% identical to the sequences set forth in Table 1 (e.g., SEQ ID NOs: 11 and/or 21, or 31 and/or 41). An antibody having a full length heavy chain and full length light chain having high (e.g., 80% or greater) identity to the full length heavy chains of any of SEQ ID NOs: 11 or 31, and full length light chains of any of SEQ ID NOs: 21 or 41, can be obtained by mutagenesis (e.g., site-directed or PCR-mediated mutagenesis) of nucleic acid molecules encoding such polypeptides, followed by testing of the encoded altered antibody for retained function using the functional assays described herein.

In one aspect, provided herein is an isolated antibody, or a functional antigen binding fragment thereof, comprising a heavy chain and a light chain, wherein the heavy chain comprises an amino acid sequence that is at least 80%, at least 90%, or at least 95% identical to an amino acid sequence selected from the group consisting of SEQ ID NOs: 11 and 31; the light chain comprises an amino acid sequence that is at least 80%, at least 90%, or at least 95% identical to an amino acid sequence selected from the group consisting of SEQ ID NOs: 21 and 41; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain and a light chain, wherein the heavy chain comprises an amino acid sequence that is at least 80%, at least 90%, or at least 95% identical to the amino acid sequence of SEQ ID NO: 11; the light chain comprises an amino acid sequence that is at least 80%, at least 90%, or at least 95% identical to the amino acid sequence of SEQ ID NO: 21; and the antibody specifically binds to FXI and/or FXIa (e.g., human, rabbit, cynomolgus monkey, and baboon FXIa). In one embodiment, an isolated antibody, or a functional antigen binding fragment thereof, comprises a heavy chain and a light chain, wherein the heavy chain comprises an amino acid sequence that is at least 80%, at least 90%, or at least 95% identical to the amino acid sequence of SEQ ID NO: 31; the light chain comprises an amino acid sequence that is at least 80%, at least 90%, or at least 95% identical to the amino acid sequence of SEQ ID NO: 41; and the antibody specifically binds to FXI and/or FXIa (e.g., human, rabbit, cynomolgus monkey, and baboon FXIa). In certain aspects of the present disclosure the heavy and light chain sequences further comprise HCDR1, HCDR2, HCDR3, LCDR1, LCDR2, and LCDR3 sequences as defined by Kabat, for example SEQ ID NOs: 3, 4, 5, 13, 14, and 15, respectively. In certain other aspects of the present disclosure the heavy and light chain sequences further comprise HCDR1, HCDR2, HCDR3, LCDR1, LCDR2, and LCDR3 sequences as defined by Chothia, for example SEQ ID NOs: 6, 7, 8, 16, 17, and 18, respectively. In certain other aspects, the heavy and light chain sequences further comprise HCDR1, HCDR2, HCDR3, LCDR1, LCDR2, and LCDR3 sequences as defined by the Combined system, for example SEQ ID NOs: 46, 4, 5, 33, 14, and 15, respectively. In certain other aspects, the heavy and light chain sequences further comprise HCDR1, HCDR2, HCDR3, LCDR1, LCDR2, and LCDR3 sequences as defined by IMGT, for example SEQ ID NOs: 43, 44, 45, 47, 37, and 15, respectively.

In other embodiments for use in the methods described herein, the full length heavy chain and/or full length light chain nucleotide sequences may be 60%, 70%, 80%, 90%, 95%, 96%, 97%, 98% or 99% identical to the sequences set forth in Table 1 (e.g., SEQ ID NOs: 12 and/or 22, or 32 and/or 42).

In other embodiments for use in the methods described herein, the variable regions of heavy chain and/or the variable regions of light chain nucleotide sequences may be 60%, 70%, 80%, 90%, 95%, 96%, 97%, 98% or 99% identical to the sequences set forth in Table 1 (e.g., SEQ ID NOs: 10 and/or 20, or 30 and/or 40). In other embodiments for use in the formulations described herein, the variable regions of heavy chain and/or the variable regions of light chain nucleotide sequences may be 60%, 70%, 80%, 90%, 95%, 96%, 97%, 98% or 99% identical to the sequences set forth in Table 1 (e.g., SEQ ID NOs: 10 and/or 20, or 30 and/or 40).

As used herein, the percent identity between the two sequences is a function of the number of identical positions shared by the sequences (i.e., % identity equals number of identical positions/total number of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The isolated anti-FXI and/or FXIa antibodies, or antigen binding fragments thereof, as described herein can be monoclonal antibodies, human or humanized antibodies, chimeric antibodies, single chain antibodies, Fab fragments, Fv fragments, F(ab′)2 fragments, or scFv fragments, and/or IgG isotypes (e.g., IgG1 such as human IgG1). In specific embodiments, anti-FXI and/or anti-FXIa antibodies described herein are recombinant human antibodies. In specific embodiments, anti-FXI and/or anti-FXIa antibodies described herein are human IgG1/lambda (λ) antibodies. In specific embodiments, anti-FXI and/or anti-FXIa antibodies described herein are human IgG1/lambda (λ) antibodies comprising an Fc domain engineered to reduce the potential for effector function (e.g., ADCC and/or CDC), for example a human Fc domain comprising D265A and/or P329A substitutions.

Additionally or alternatively, the protein sequences of the present disclosure can further be used as a “query sequence” to perform a search against public databases to, for example, identify related sequences. For example, such searches can be performed using the BLAST program (version 2.0) of Altschul, et al., 1990 J. Mol. Biol. 215:403-10.

Antibodies with Conservative Modifications

In certain other embodiments, an antibody of the present disclosure for use in the methods described herein (e.g., methods for treating a subject afflicted with or at risk of developing a thromboembolic disorder) has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the FXIa-binding antibodies of the present disclosure. In certain other embodiments, an antibody of the present disclosure for use in the formulations described herein (e.g., the formulation in the vial, the intravenous drug delivery formulation) has a heavy chain variable region comprising CDR1, CDR2, and CDR3 sequences and a light chain variable region comprising CDR1, CDR2, and CDR3 sequences, wherein one or more of these CDR sequences have specified amino acid sequences based on the antibodies described herein or conservative modifications thereof, and wherein the antibodies retain the desired functional properties of the FXIa-binding antibodies of the present disclosure.

Accordingly, for use in the methods described herein, in some embodiments the present disclosure provides an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 3 and 23, and conservative modifications thereof; the heavy chain variable region CDR2 amino acid sequences are selected from the group consisting of SEQ ID NOs: 4 and 24, and conservative modifications thereof; the heavy chain variable region CDR3 amino acid sequences are selected from the group consisting of SEQ ID NOs: 5 and 25, and conservative modifications thereof; the light chain variable regions CDR1 amino acid sequences are selected from the group consisting of SEQ ID NOs: 13 and 33, and conservative modifications thereof; the light chain variable regions CDR2 amino acid sequences are selected from the group consisting of SEQ ID NOs: 14 and 34, and conservative modifications thereof; the light chain variable regions of CDR3 amino acid sequences are selected from the group consisting of SEQ ID NOs: 15 and 35, and conservative modifications thereof; and the antibody or antigen binding fragments thereof specifically binds to FXIa.

For use in the formulations described herein, in some embodiments the present disclosure provides an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of SEQ ID NOs: 3 and 23, and conservative modifications thereof; the heavy chain variable region CDR2 amino acid sequences are selected from the group consisting of SEQ ID NOs: 4 and 24, and conservative modifications thereof; the heavy chain variable region CDR3 amino acid sequences are selected from the group consisting of SEQ ID NOs: 5 and 25, and conservative modifications thereof; the light chain variable regions CDR1 amino acid sequences are selected from the group consisting of SEQ ID NOs: 13 and 33, and conservative modifications thereof; the light chain variable regions CDR2 amino acid sequences are selected from the group consisting of SEQ ID NOs: 14 and 34, and conservative modifications thereof; the light chain variable regions of CDR3 amino acid sequences are selected from the group consisting of SEQ ID NOs: 15 and 35, and conservative modifications thereof; and the antibody or antigen binding fragments thereof specifically binds to FXIa.

In one aspect, provided herein is an isolated antibody, or an antigen binding fragment thereof, consisting of a heavy chain variable region comprising CDR1, CDR2, and CDR3 sequences and a light chain variable region comprising CDR1, CDR2, and CDR3 sequences, wherein: the heavy chain variable region CDR1 amino acid sequences are selected from the group consisting of those described in Table 1, and conservative modifications thereof; the heavy chain variable region CDR2 amino acid sequences are selected from the group consisting of those described in Table 1, and conservative modifications thereof; the heavy chain variable region CDR3 amino acid sequences are selected from the group consisting of those described in Table 1, and conservative modifications thereof; the light chain variable regions CDR1 amino acid sequences are selected from the group consisting of those described in Table 1, and conservative modifications thereof; the light chain variable regions CDR2 amino acid sequences are selected from the group consisting of those described in Table 1, and conservative modifications thereof; the light chain variable regions of CDR3 amino acid sequences are selected from the group consisting of those described in Table 1, and conservative modifications thereof; and the antibody or antigen binding fragments thereof specifically binds to FXIa.

In other embodiments for use in the methods described herein, the antibody of the present disclosure is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the FXIa binding antibodies of the present disclosure. In other embodiments for use in the formulations described herein, the antibody of the present disclosure is optimized for expression in a mammalian cell has a full length heavy chain sequence and a full length light chain sequence, wherein one or more of these sequences have specified amino acid sequences based on the antibodies described herein or conservative modifications thereof, and wherein the antibodies retain the desired functional properties of the FXIa binding antibodies of the present disclosure. Accordingly, the present disclosure provides an isolated antibody optimized for expression in a mammalian cell consisting of a full length heavy chain and a full length light chain wherein the full length heavy chain has amino acid sequences selected from the group of SEQ ID NOs: 11 or 31, and conservative modifications thereof; and the full length light chain has amino acid sequences selected from the group of SEQ ID NOs: 21 or 41, and conservative modifications thereof; and the antibody specifically binds to FXI and/or FXIa (e.g., human, rabbit, cynomolgus monkey, and baboon FXIa).

Antibodies that Bind to the Same Epitope

In some embodiments, the present disclosure provides antibodies that compete for the same epitope as the FXI and/or FXIa binding antibodies described in Table 1, for use in the methods described herein (e.g., methods for treating a subject afflicted with or at risk of developing a thromboembolic disorder). In some embodiments, the present disclosure provides antibodies that compete for the same epitope as the FXI and/or FXIa binding antibodies described in Table 1, for use in the formulations described herein (e.g., the formulation in the vial, the intravenous drug delivery formulation). Additional antibodies can therefore be identified based on their ability to compete (e.g., to competitively inhibit the binding of, in a statistically significant manner, by binding to the same or overlapping epitope) with other antibodies of the present disclosure in FXI and/or FXIa binding assays (such as those described in the Examples Section). The ability of a test antibody to inhibit the binding of antibodies of the present disclosure to a FXI and/or FXIa protein demonstrates that the test antibody can compete with that antibody for binding to FXI and/or FXIa; such an antibody may, according to non-limiting theory, bind to the same or a related (e.g., a structurally similar or spatially proximal) epitope on the FXI and/or FXIa protein as the antibody with which it competes. In a certain embodiment, the antibody that binds to the same epitope on FXI and/or FXIa as the antibodies of the present disclosure is a human monoclonal antibody. Such human monoclonal antibodies can be prepared and isolated as described herein.

As used herein, an antibody “competes” for binding when the competing antibody binds to the same FXI and/or FXIa epitope as an antibody or antigen binding fragment of the present disclosure (e.g., Antibody 1 or Antibody 2) and inhibits FXI and/or FXIa binding of an antibody or antigen binding fragment of the present disclosure by more than 50% (for example, 80%, 85%, 90%, 95%, 98% or 99%) in the presence of an equimolar concentration of competing antibody. This may be determined, for instance, in a competitive binding assay, by any of the methods well known to those of skill in the art.

As used herein, an antibody or antigen binding fragment thereof does not “compete” with a FXI and/or FXIa antibody or antigen binding fragment of the present disclosure (e.g., Antibody 1 or Antibody 2) unless said competing antibody or antigen binding fragment thereof binds the same FXI and/or FXIa epitope, or an overlapping FXI and/or FXIa epitope, as an antibody or antigen binding fragment of the present disclosure. As used herein, a competing antibody or antigen binding fragment thereof does not include one which (i) sterically blocks an antibody or antigen binding fragment of the present disclosure from binding its target (e.g., if said competing antibody binds to a nearby, non-overlapping FXI and/or FXIa epitope and physically prevents an antibody or antigen binding fragment of the present disclosure from binding its target); and/or (ii) binds to a different, non-overlapping FXI and/or FXIa epitope and induces a conformational change to the FXI and/or FXIa protein such that said protein can no longer be bound by a FXI and/or FXIa antibody or antigen binding fragment of the present disclosure in a way that would occur absent said conformational change.

### Engineered and Modified Antibodies

In some embodiments, an antibody of the present disclosure, for use in the methods described herein, further can be prepared using an antibody having one or more of the VH and/or VL sequences shown herein as starting material to engineer a modified antibody, which modified antibody may have altered properties from the starting antibody. In some embodiments, an antibody of the present disclosure, for use in the formulations described herein, further can be prepared using an antibody having one or more of the VH and/or VL sequences shown herein as starting material to engineer a modified antibody, which modified antibody may have altered properties from the starting antibody. An antibody can be engineered by modifying one or more residues within one or both variable regions (i.e., VH and/or VL), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann, L. et al., 1998 Nature 332:323-327; Jones, P. et al., 1986 Nature 321:522-525; Queen, C. et al., 1989 Proc. Natl. Acad., U.S.A. 86:10029-10033; U.S. Pat. No. 5,225,539 to Winter, and U.S. Pat. Nos. 5,530,101; 5,585,089; 5,693,762 and 6,180,370 to Queen et al.)

Accordingly, another embodiment of the present disclosure pertains to an isolated antibody, or an antigen binding fragment thereof, comprising a heavy chain variable region comprising CDR1 sequences having an amino acid sequence selected from the group consisting of SEQ ID NOs: 3 and 23; CDR2 sequences having an amino acid sequence selected from the group consisting of SEQ ID NOs: 4 and 24; CDR3 sequences having an amino acid sequence selected from the group consisting of SEQ ID NOs: 5 and 25, respectively; and a light chain variable region having CDR1 sequences having an amino acid sequence selected from the group consisting of SEQ ID NOs: 13 and 33; CDR2 sequences having an amino acid sequence selected from the group consisting of SEQ ID NOs: 14 and 34; and CDR3 sequences consisting of an amino acid sequence selected from the group consisting of SEQ ID NOs: 15 and 35, respectively. Thus, such antibodies contain the VH and VL CDR sequences of monoclonal antibodies, yet may contain different framework sequences from these antibodies.

Such framework sequences can be obtained from public DNA databases or published references that include germline antibody gene sequences. For example, germline DNA sequences for human heavy and light chain variable region genes can be found in the “VBase” human germline sequence database, as well as in Kabat, E. A., et al., 1991 Sequences of Proteins of Immunological Interest, Fifth Edition, U.S. Department of Health and Human Services, NIH Publication No. 91-3242; Tomlinson, I. M., et al., 1992 J. Mol. Biol. 227:776-798; and Cox, J. P. L. et al., 1994 Eur. J Immunol. 24:827-836; the contents of each of which are expressly incorporated herein by reference.

An example of framework sequences for use in the antibodies of the present disclosure are those that are structurally similar to the framework sequences used by selected antibodies of the present disclosure, e.g., consensus sequences and/or framework sequences used by monoclonal antibodies of the present disclosure. The VH CDR1, 2 and 3 sequences, and the VL CDR1, 2 and 3 sequences, can be grafted onto framework regions that have the identical sequence as that found in the germline immunoglobulin gene from which the framework sequence derive, or the CDR sequences can be grafted onto framework regions that contain one or more mutations as compared to the germline sequences. For example, it has been found that in certain instances it is beneficial to mutate residues within the framework regions to maintain or enhance the antigen binding ability of the antibody (see e.g., U.S. Pat. Nos. 5,530,101; 5,585,089; 5,693,762 and 6,180,370 to Queen et al). Frameworks that can be utilized as scaffolds on which to build the antibodies and antigen binding fragments described herein include, but are not limited to VH1A, VH1B, VH3, Vk1, V12, and Vk2.

Accordingly, for use in the methods described herein, another embodiment of the present disclosure relates to isolated FXIa binding antibodies, or antigen binding fragments thereof, comprising a heavy chain variable region comprising an amino acid sequence selected from the group consisting of SEQ ID NOs: 9 and 29, or an amino acid sequence having one, two, three, four or five amino acid substitutions, deletions or additions in the framework region of such sequences, and further comprising a light chain variable region having an amino acid sequence selected from the group consisting of SEQ ID NOs: 19 and 39, or an amino acid sequence having one, two, three, four or five amino acid substitutions, deletions or additions in the framework region of such sequences.

Accordingly, for use in the formulations described herein, another embodiment of the present disclosure relates to isolated FXIa binding antibodies, or antigen binding fragments thereof, comprising a heavy chain variable region comprising an amino acid sequence selected from the group consisting of SEQ ID NOs: 9 and 29, or an amino acid sequence having one, two, three, four or five amino acid substitutions, deletions or additions in the framework region of such sequences, and further comprising a light chain variable region having an amino acid sequence selected from the group consisting of SEQ ID NOs: 19 and 39, or an amino acid sequence having one, two, three, four or five amino acid substitutions, deletions or additions in the framework region of such sequences.

Another type of variable region modification is mutation of amino acid residues within the VH and/or VL CDR1, CDR2 and/or CDR3 regions to thereby improve one or more binding properties (e.g., affinity) of the antibody of interest, known as “affinity maturation.” Site-directed mutagenesis or PCR-mediated mutagenesis can be performed to introduce the mutation(s) and the effect on antibody binding, or other functional property of interest, can be evaluated in in vitro or in vivo assays as described herein and provided in the Examples Section. Conservative modifications (as discussed above) can be introduced. The mutations may be amino acid substitutions, additions or deletions. Moreover, typically no more than one, two, three, four or five residues within a CDR region are altered.

Accordingly, in another embodiment for use in the methods described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 3 and 23 or an amino acid sequence having one, two, three, four or five amino acid substitutions, deletions or additions as compared to SEQ ID NOs: 3 and 23; a VH CDR2 region having an amino acid sequence selected from the group consisting of SEQ ID NOs: 4 and 24 or an amino acid sequence having one, two, three, four or five amino acid substitutions, deletions or additions as compared to SEQ ID NOs: 4 and 24; a VH CDR3 region having an amino acid sequence selected from the group consisting of SEQ ID NOs: 5 and 25, or an amino acid sequence having one, two, three, four or five amino acid substitutions, deletions or additions as compared to SEQ ID NOs: 5 and 25; a VL CDR1 region having an amino acid sequence selected from the group consisting of SEQ ID NOs: 13 and 33, or an amino acid sequence having one, two, three, four or five amino acid substitutions, deletions or additions as compared to SEQ ID NOs: 13 and 33; a VL CDR2 region having an amino acid sequence selected from the group consisting of SEQ ID NOs: 14 and 34, or an amino acid sequence having one, two, three, four or five amino acid substitutions, deletions or additions as compared to SEQ ID NOs: 14 and 34; and a VL CDR3 region having an amino acid sequence selected from the group consisting of SEQ ID NOs: 15 and 35, or an amino acid sequence having one, two, three, four or five amino acid substitutions, deletions or additions as compared to SEQ ID NOs: 15 and 35.

Accordingly, in another embodiment for use in the methods described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 6 and 26 or an amino acid sequence having one, two, three, four or five amino acid substitutions, deletions or additions as compared to SEQ ID NOs: 6 and 26; a VH CDR2 region having an amino acid sequence selected from the group consisting of SEQ ID NOs: 7 and 27 or an amino acid sequence having one, two, three, four or five amino acid substitutions, deletions or additions as compared to SEQ ID NOs: 7 and 27; a VH CDR3 region having an amino acid sequence selected from the group consisting of SEQ ID NOs: 8 and 28, or an amino acid sequence having one, two, three, four or five amino acid substitutions, deletions or additions as compared to SEQ ID NOs: 8 and 28; a VL CDR1 region having an amino acid sequence selected from the group consisting of SEQ ID NOs: 16 and 36, or an amino acid sequence having one, two, three, four or five amino acid substitutions, deletions or additions as compared to SEQ ID NOs: 16 and 36; a VL CDR2 region having an amino acid sequence selected from the group consisting of SEQ ID NOs: 17 and 37, or an amino acid sequence having one, two, three, four or five amino acid substitutions, deletions or additions as compared to SEQ ID NOs: 17 and 37; and a VL CDR3 region having an amino acid sequence selected from the group consisting of SEQ ID NOs: 18 and 38, or an amino acid sequence having one, two, three, four or five amino acid substitutions, deletions or additions as compared to SEQ ID NOs: 18 and 38.

Accordingly, in another embodiment for use in the formulations described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 3 and 23 or an amino acid sequence having one, two, three, four or five amino acid substitutions, deletions or additions as compared to SEQ ID NOs: 3 and 23; a VH CDR2 region having an amino acid sequence selected from the group consisting of SEQ ID NOs: 4 and 24 or an amino acid sequence having one, two, three, four or five amino acid substitutions, deletions or additions as compared to SEQ ID NOs: 4 and 24; a VH CDR3 region having an amino acid sequence selected from the group consisting of SEQ ID NOs: 5 and 25, or an amino acid sequence having one, two, three, four or five amino acid substitutions, deletions or additions as compared to SEQ ID NOs: 5 and 25; a VL CDR1 region having an amino acid sequence selected from the group consisting of SEQ ID NOs: 13 and 33, or an amino acid sequence having one, two, three, four or five amino acid substitutions, deletions or additions as compared to SEQ ID NOs: 13 and 33; a VL CDR2 region having an amino acid sequence selected from the group consisting of SEQ ID NOs: 14 and 34, or an amino acid sequence having one, two, three, four or five amino acid substitutions, deletions or additions as compared to SEQ ID NOs: 14 and 34; and a VL CDR3 region having an amino acid sequence selected from the group consisting of SEQ ID NOs: 15 and 35, or an amino acid sequence having one, two, three, four or five amino acid substitutions, deletions or additions as compared to SEQ ID NOs: 15 and 35.

Accordingly, in another embodiment for use in the formulations described herein, the present disclosure provides isolated FXIa-binding antibodies, or antigen binding fragments thereof, consisting of a heavy chain variable region having a VH CDR1 region consisting of an amino acid sequence selected from the group having SEQ ID NOs: 6 and 26 or an amino acid sequence having one, two, three, four or five amino acid substitutions, deletions or additions as compared to SEQ ID NOs: 6 and 26; a VH CDR2 region having an amino acid sequence selected from the group consisting of SEQ ID NOs: 7 and 27 or an amino acid sequence having one, two, three, four or five amino acid substitutions, deletions or additions as compared to SEQ ID NOs: 7 and 27; a VH CDR3 region having an amino acid sequence selected from the group consisting of SEQ ID NOs: 8 and 28, or an amino acid sequence having one, two, three, four or five amino acid substitutions, deletions or additions as compared to SEQ ID NOs: 8 and 28; a VL CDR1 region having an amino acid sequence selected from the group consisting of SEQ ID NOs: 16 and 36, or an amino acid sequence having one, two, three, four or five amino acid substitutions, deletions or additions as compared to SEQ ID NOs: 16 and 36; a VL CDR2 region having an amino acid sequence selected from the group consisting of SEQ ID NOs: 17 and 37, or an amino acid sequence having one, two, three, four or five amino acid substitutions, deletions or additions as compared to SEQ ID NOs: 17 and 37; and a VL CDR3 region having an amino acid sequence selected from the group consisting of SEQ ID NOs: 18 and 38, or an amino acid sequence having one, two, three, four or five amino acid substitutions, deletions or additions as compared to SEQ ID NOs: 18 and 38.

Antibodies with Extended Half Life

In some embodiments, the present disclosure provides for antibodies that specifically bind to FXIa protein which have an extended half-life in vivo, for use in the methods or formulations described herein.

Many factors may affect a protein's half-life in vivo. For examples, kidney filtration, metabolism in the liver, degradation by proteolytic enzymes (proteases), and immunogenic responses (e.g., protein neutralization by antibodies and uptake by macrophages and dendritic cells). A variety of strategies can be used to extend the half-life of the antibodies of the present disclosure. For example, by chemical linkage to polyethyleneglycol (PEG), reCODE PEG, antibody scaffold, polysialic acid (PSA), hydroxyethyl starch (HES), albumin-binding ligands, and carbohydrate shields; by genetic fusion to proteins binding to serum proteins, such as albumin, IgG, FcRn, and transferring; by coupling (genetically or chemically) to other binding moieties that bind to serum proteins, such as nanobodies, Fabs, DARPins, avimers, affibodies, and anticalins; by genetic fusion to rPEG, albumin, domain of albumin, albumin-binding proteins, and Fc; or by incorporation into nanocarriers, slow release formulations, or medical devices.

To prolong the serum circulation of antibodies in vivo, inert polymer molecules such as high molecular weight PEG can be attached to the antibodies or a fragment thereof with or without a multifunctional linker either through site-specific conjugation of the PEG to the N- or C-terminus of the antibodies or via epsilon-amino groups present on lysine residues. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. The pegylation can be carried out by an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein. Methods for pegylating proteins are known in the art and can be applied to the antibodies of the present disclosure. See for example, EP 0 154 316 by Nishimura et al. and EP 0 401 384 by Ishikawa et al.

Other modified pegylation technologies include reconstituting chemically orthogonal directed engineering technology (ReCODE PEG), which incorporates chemically specified side chains into biosynthetic proteins via a reconstituted system that includes tRNA synthetase and tRNA. This technology enables incorporation of more than 30 new amino acids into biosynthetic proteins in E. coli, yeast, and mammalian cells. The tRNA incorporates a nonnative amino acid any place an amber codon is positioned, converting the amber from a stop codon to one that signals incorporation of the chemically specified amino acid.

Recombinant pegylation technology (rPEG) can also be used for serum halflife extension. This technology involves genetically fusing a 300-600 amino acid unstructured protein tail to an existing pharmaceutical protein. Because the apparent molecular weight of such an unstructured protein chain is about 15-fold larger than its actual molecular weight, the serum half-life of the protein is greatly increased. In contrast to traditional PEGylation, which requires chemical conjugation and repurification, the manufacturing process is greatly simplified and the product is homogeneous.

Polysialytion is another technology, which uses the natural polymer polysialic acid (PSA) to prolong the active life and improve the stability of therapeutic peptides and proteins. PSA is a polymer of sialic acid (a sugar). When used for protein and therapeutic peptide drug delivery, polysialic acid provides a protective microenvironment on conjugation. This increases the active life of the therapeutic protein in the circulation and prevents it from being recognized by the immune system. The PSA polymer is naturally found in the human body. It was adopted by certain bacteria which evolved over millions of years to coat their walls with it. These naturally polysialylated bacteria were then able, by virtue of molecular mimicry, to foil the body's defense system. PSA, nature's ultimate stealth technology, can be easily produced from such bacteria in large quantities and with predetermined physical characteristics. Bacterial PSA is completely non-immunogenic, even when coupled to proteins, as it is chemically identical to PSA in the human body.

Another technology includes the use of hydroxyethyl starch (“HES”) derivatives linked to antibodies. HES is a modified natural polymer derived from waxy maize starch and can be metabolized by the body's enzymes. HES solutions are usually administered to substitute deficient blood volume and to improve the rheological properties of the blood. Hesylation of an antibody enables the prolongation of the circulation half-life by increasing the stability of the molecule, as well as by reducing renal clearance, resulting in an increased biological activity. By varying different parameters, such as the molecular weight of HES, a wide range of HES antibody conjugates can be customized.

Antibodies having an increased half-life in vivo can also be generated introducing one or more amino acid modifications (i.e., substitutions, insertions or deletions) into an IgG constant domain, or FcRn binding fragment thereof (preferably a Fc or hinge Fc domain fragment). See, e.g., International Publication No. WO 98/23289; International Publication No. WO 97/34631; and U.S. Pat. No. 6,277,375.

Further, antibodies can be conjugated to albumin (e.g., human serum albumin; HSA) in order to make the antibody or antibody fragment more stable in vivo or have a longer half-life in vivo. The techniques are well-known in the art, see, e.g., International Publication Nos. WO 93/15199, WO 93/15200, and WO 01/77137; and European Patent No. EP 413,622. In addition, in the context of a bispecific antibody as described above, the specificities of the antibody can be designed such that one binding domain of the antibody binds to FXIa while a second binding domain of the antibody binds to serum albumin, preferably HSA.

The strategies for increasing half-life is especially useful in nanobodies, fibronectin-based binders, and other antibodies or proteins for which increased in vivo half-life is desired.

### Antibody Conjugates

In some embodiments, the present disclosure provides antibodies or fragments thereof, for use in the methods or formulations described herein, that specifically bind to an FXIa protein recombinantly fused or chemically conjugated (including both covalent and non-covalent conjugations) to a heterologous protein or polypeptide (or fragment thereof, preferably to a polypeptide of at least 10, at least 20, at least 30, at least 40, at least 50, at least 60, at least 70, at least 80, at least 90 or at least 100 amino acids) to generate fusion proteins. In particular, the present disclosure provides fusion proteins comprising an antigen-binding fragment of an antibody described herein (e.g., a Fab fragment, Fd fragment, Fv fragment, F(ab)2 fragment, a VH domain, a VH CDR, a VL domain or a VL CDR) and a heterologous protein, polypeptide, or peptide. Methods for fusing or conjugating proteins, polypeptides, or peptides to an antibody or an antibody fragment are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341.

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the present disclosure or fragments thereof (e.g., antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies or fragments thereof, or the encoded antibodies or fragments thereof, may be altered by being subjected to random mutagenesis by error-prone PCR, random nucleotide insertion or other methods prior to recombination. A polynucleotide encoding an antibody or fragment thereof that specifically binds to an FXIa protein may be recombined with one or more components, motifs, sections, parts, domains, fragments, etc. of one or more heterologous molecules.

Moreover, the antibodies or fragments thereof can be fused to marker sequences, such as a peptide to facilitate purification. In certain embodiments, the marker amino acid sequence is a hexa-histidine peptide (SEQ ID NO: 48), such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine (SEQ ID NO: 48) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present disclosure or fragments thereof conjugated to a diagnostic or detectable agent. Such antibodies can be useful for monitoring or prognosing the onset, development, progression and/or severity of a disease or disorder as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidinlbiotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (131I, 125I, 123I, and 121I), carbon (14C), sulfur (35S), tritium (3H), indium (115In, 113In, 112In, and 111In,), technetium (99Tc), thallium (201Ti), gallium (68Ga, 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142 Pr, 105Rh, 97Ru, 68Ge, 57Co, 65Zn, 85Sr, 32P, 153Gd, 169Yb, 51Cr, 54Mn, 75Se, 113Sn, and 117Tin; and positron emitting metals using various positron emission tomographies, and noradioactive paramagnetic metal ions.

In some embodiments, the present disclosure further encompasses uses of antibodies or fragments thereof conjugated to a therapeutic moiety. An antibody or fragment thereof may be conjugated to a therapeutic moiety such as a cytotoxin, e.g., a cytostatic or cytocidal agent, a therapeutic agent or a radioactive metal ion, e.g., alpha-emitters. A cytotoxin or cytotoxic agent includes any agent that is detrimental to cells.

Further, an antibody or fragment thereof may be conjugated to a therapeutic moiety or drug moiety that modifies a given biological response. Therapeutic moieties or drug moieties are not to be construed as limited to classical chemical therapeutic agents. For example, the drug moiety may be a protein, peptide, or polypeptide possessing a desired biological activity. Such proteins may include, for example, a toxin such as abrin, ricin A, pseudomonas exotoxin, cholera toxin, or diphtheria toxin; a protein such as tumor necrosis factor, α-interferon, β-interferon, nerve growth factor, platelet derived growth factor, tissue plasminogen activator, an apoptotic agent, an anti-angiogenic agent; or, a biological response modifier such as, for example, a lymphokine.

Moreover, an antibody can be conjugated to therapeutic moieties such as a radioactive metal ion, such as alph-emiters such as 213Bi or macrocyclic chelators useful for conjugating radiometal ions, including but not limited to, 131In, 131LU, 131Y, 131Ho, 131Sm, to polypeptides. In certain embodiments, the macrocyclic chelator is 1,4,7,10-tetraazacyclododecane-N,N′,N″,N′″-tetraacetic acid (DOTA) which can be attached to the antibody via a linker molecule. Such linker molecules are commonly known in the art and described in Denardo et al., 1998, Clin Cancer Res. 4(10):2483-90; Peterson et al., 1999, Bioconjug. Chem. 10(4):553-7; and Zimmerman et al., 1999, Nucl. Med. Biol. 26(8):943-50, each incorporated by reference in their entireties.

Techniques for conjugating therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies 84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985), and Thorpe et al., 1982, Immunol. Rev. 62:119-58.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### Pharmaceutical Formulations

In some embodiments, the present disclosure also provides pharmaceutical formulations that contain a therapeutically effective amount of a Factor XI and/or Factor XIa antibody disclosed herein (e.g., Antibody 1). The pharmaceutical formulation comprises one or more excipients and is maintained at a certain pH. Non-limiting examples of an “excipient,” as used herein, include any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

### Drug Substance

Antibody 1 is a high-affinity, anti-human Factor XI monoclonal antibody. It is expressed in a Chinese hamster ovary cell line (CHO-C8TD). In some embodiments, the Antibody 1 drug substance is fully formulated for subcutaneous administration (i.e., no further excipients are added), and thus is identical in composition to the Antibody 1 drug product. In some embodiments, for intravenous administration, the Antibody 1 drug product is further diluted in an appropriate carrier. In some embodiments, the Antibody 1 drug product is diluted in a solution comprising dextrose, e.g., dextrose 5% in water (D5W).

### Excipients and pH

In some embodiments the excipients contained in the Antibody 1 drug product are pharmacopoeial grade excipients. In some embodiments, the excipients in the Antibody 1 drug product comprise a histidine, a histidine salt, a sugar, and a polysorbate. In some embodiments, the excipients in the Antibody 1 drug product include L-histidine and L-histidine hydrochloride monohydrate (histidine buffer), sucrose, and polysorbate 20. Excipients may be selected for their suitability for intravenous and subcutaneous administration, providing the necessary stabilizing, buffering capacity, and tonicity. The formulation maximizes the stability of the monoclonal antibody product, and may provide a sterile solution suitable for subcutaneous or intravenous administration. In some embodiments, a sugar (e.g., sucrose) acts as a stabilizer. In some embodiments, a histidine (e.g., L-histidine, L-Histidine HCl monohydrate) acts as a buffering agent. In some embodiments, a polysorbate (e.g., polysorbate 20), acts as a stabilizer. In some embodiments, the formulation is adjusted to final volume in water for injection (WFI).

The one or more excipients in the pharmaceutical formulation of the present invention comprises a buffering agent. The term “buffering agent,” as used herein, refers to one or more components that when added to an aqueous solution is able to protect the solution against variations in pH when adding acid or alkali, or upon dilution with a solvent. In addition to phosphate buffers, glycinate, carbonate, citrate, histidine buffers and the like can be used, in which case, sodium, potassium or ammonium ions can serve as counterion.

In certain embodiments, the buffer or buffer system comprises at least one buffer that has a buffering range that overlaps fully or in part with the range of pH 5.0-7.4. In certain embodiments, the buffer has a pH of about 5.5±0.5. In certain embodiments, the buffer comprises a histidine buffer. In certain embodiments, the histidine buffer is present at a concentration of 0.05-10 mM, 0.1-10 mM, 0.2-10 mM, 0.5-10 mM, 1-10 mM, 5-10 mM, 5 to 100 mM, 10 to 100 mM, 15 to 100 mM, 20 to 100 mM, 30 to 100 mM, 40 to 100 mM, 50 to 100 mM, 60 to 100 mM, 70 to 100 mM, 80 to 100 mM, 90 to 100 mM, 5 to 90 mM, 5 to 80 mM, 5 to 70 mM, 5 to 60 mM, 5 to 50 mM, 5 to 40 mM, 5 to 30 mM, 5 to 20 mM, 10 to 50 mM, 10 to 40 mM, 10 to 30 mM, 10 to 20 mM, 5 to 25 mM, 10 to 25 mM, 15 to 25 mM, 20 to 25 mM, 5 to 20 mM, 10 to 20 mM, or 15 to 20 mM. In certain embodiments, the histidine is present at a concentration of about 0.1 mM, 0.2 mM, 0.5 mM, 1 mM, 5 mM, about 10 mM, about 15 mM, about 20 mM, about 25 mM, or about 50 mM. In certain embodiments, the histidine buffer is present at a concentration of about 20 mM. In certain embodiments, the histidine buffer is present at a concentration of about 0.20 mM. In certain embodiments, the histidine buffer has a pH of about 5.0, about 5.5, about 6.0, about 6.5, or about 7.0. In a particular embodiment, the histidine buffer has a pH of about 5.5.

The pharmaceutical formulation of the present invention may have a pH of 5.0 to 6.0. For example, in certain embodiments, the pharmaceutical formulation has a pH of 5.0 to 6.0 (i.e., 5.5±0.5), 5.1 to 5.9 (i.e., 5.5±0.4), 5.2 to 5.8 (i.e., 5.5±0.3), 5.3 to 5.7 (i.e., 5.5±0.2), 5.4 to 5.6 (i.e., 5.5±0.1), or 5.45 to 5.55 (i.e., 5.5±0.05). In certain embodiments, the pharmaceutical formulation has a pH of about 5.5, about 5.6, about 5.7, about 5.8, about 5.9, about 6.0, about 6.1, about 6.2, about 6.3, about 6.4, or about 6.5. In certain embodiments, the pharmaceutical formulation has a pH of about 5.5. Under the rules of scientific rounding, a pH greater than or equal to 5.45 and smaller than or equal to 5.55 is rounded as 5.5.

In certain embodiments, the buffer system of the pharmaceutical formulation comprises histidine at 10 to 30 mM, at a pH of 5.5±0.2. In certain embodiments, the buffer system of the pharmaceutical formulation comprises histidine at about 20 mM, at a pH of 5.5±0.2. In certain embodiments, the buffer system of the pharmaceutical formulation comprises histidine at 10 to 30 mM, at a pH of 5.5±0.05. In certain embodiments, the buffer system of the pharmaceutical formulation comprises histidine at about 20 mM, at a pH of 5.5±0.05.

In certain embodiments, the buffer system of the pharmaceutical formulation comprises histidine at 0.10 to 0.30 mM, at a pH of 5.5±0.2. In certain embodiments, the buffer system of the pharmaceutical formulation comprises histidine at about 0.20 mM, at a pH of 5.5±0.2. In certain embodiments, the buffer system of the pharmaceutical formulation comprises histidine at 0.10 to 0.30 mM, at a pH of 5.5±0.05. In certain embodiments, the buffer system of the pharmaceutical formulation comprises histidine at about 0.20 mM, at a pH of 5.5±0.05.

The one or more excipients in the pharmaceutical formulation of the present invention further comprises a sugar or sugar alcohol. Sugars and sugar alcohols are useful in pharmaceutical formulations as a thermal stabilizer. In certain embodiments, the pharmaceutical formulation comprises a sugar, for example, a monosaccharide (glucose, xylose, or erythritol), a disaccharide (e.g., sucrose, trehalose, maltose, or galactose), or an oligosaccharide (e.g., stachyose). In specific embodiments, the pharmaceutical formulation comprises sucrose. In certain embodiments, the pharmaceutical composition comprises a sugar alcohol, for example, a sugar alcohol derived from a monosaccharide (e.g., mannitol, sorbitol, or xylitol), a sugar alcohol derived from a disaccharide (e.g., lactitol or maltitol), or a sugar alcohol derived from an oligosaccharide. In specific embodiments, the pharmaceutical formulation comprises sucrose.

The amount of the sugar or sugar alcohol contained within the formulation can vary depending on the specific circumstances and intended purposes for which the formulation is used. In certain embodiments, the pharmaceutical formulation comprises 50 to 300 mM, 50 to 250 mM, 100 to 300 mM, 100 to 250 mM, 150 to 300 mM, 150 to 250 mM, 200 to 300 mM, 200 to 250 mM, or 250 to 300 mM of the sugar or sugar alcohol. In certain embodiments, the pharmaceutical formulation comprises about 50 mM, about 75 mM, about 100 mM, about 125 mM, about 150 mM, about 200 mM, about 220 mM, about 250 mM, or about 300 mM of the sugar or sugar alcohol. In specific embodiments, the pharmaceutical formulation comprises about 220 mM of the sugar or sugar alcohol (e.g., sucrose).

The amount of the sugar or sugar alcohol contained within the formulation can vary depending on the specific circumstances and intended purposes for which the formulation is used. In certain embodiments, the pharmaceutical formulation comprises 0.50 to 3.00 mM, 0.50 to 2.50 mM, 1.00 to 3.00 mM, 1.00 to 2.50 mM, 1.50 to 3.00 mM, 1.50 to 2.50 mM, 2.00 to 3.00 mM, 2.00 to 2.50 mM, or 2.50 to 3.00 mM of the sugar or sugar alcohol. In certain embodiments, the pharmaceutical formulation comprises about 0.50 mM, about 0.75 mM, about 1.00 mM, about 1.25 mM, about 1.50 mM, about 2.00 mM, about 2.20 mM, about 2.50 mM, or about 3.00 mM of the sugar or sugar alcohol. In specific embodiments, the pharmaceutical formulation comprises about 2.20 mM of the sugar or sugar alcohol (e.g., sucrose).

The one or more excipients in the pharmaceutical formulation disclosed herein further comprises a surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical formulations for reducing aggregation of a therapeutic protein. Surfactants suitable for use in the pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 20.

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.02% to 0.06%, 0.03% to 0.05%, or 0.035% to 0.045% of the non-ionic surfactant (e.g., polysorbate 20). In certain embodiments, the pharmaceutical formulation comprises about 0.005%, about 0.01%, about 0.02%, about 0.03%, about 0.04%, about 0.05%, about 0.06%, about 0.07%, about 0.08%, about 0.09%, or about 0.1% of the non-ionic surfactant (e.g., polysorbate 20).

The amount of a non-ionic surfactant contained within the pharmaceutical formulation of the present invention may vary depending on the specific properties desired of the formulation, as well as the particular circumstances and purposes for which the formulations are intended to be used. In certain embodiments, the pharmaceutical formulation comprises 0.0002% to 0.0006%, 0.0003% to 0.0005%, or 0.00035% to 0.00045% of the non-ionic surfactant (e.g., polysorbate 20). In certain embodiments, the pharmaceutical formulation comprises about 0.00005%, about 0.0001%, about 0.0002%, about 0.0003%, about 0.0004%, about 0.0005%, about 0.0006%, about 0.0007%, about 0.0008%, about 0.0009%, or about 0.001% of the non-ionic surfactant (e.g., polysorbate 20).

In certain embodiments, the drug product is diluted in an aqueous carrier suitable for the route of administration, e.g., intravenous administration. Exemplary carriers include sterile water for injection (SWFI), bacteriostatic water for injection (BWFI), a pH buffered solution (e.g., phosphate-buffered saline), sterile saline solution, Ringer's solution, or dextrose solution. In one embodiment, when the pharmaceutical formulation is prepared for intravenous administration, the pharmaceutical formulation can be diluted in a 5% dextrose solution (D5W).

### Exemplary Formulations

In certain embodiments, the pharmaceutical formulation of the present invention comprises an Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), histidine buffer, a sugar or sugar alcohol (e.g., sucrose), and a polysorbate (e.g., polysorbate 20), at pH 5.5 to 6.5.

In certain embodiments, the pharmaceutical formulation comprises 100 to 200 mg/mL of an Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 10 to 30 mM of histidine buffer, 200 to 300 mM of a sugar or sugar alcohol (e.g., sucrose), and 0.02% to 0.06% of a polysorbate (e.g., polysorbate 20), at pH 5.0 to 6.0. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of a sugar or sugar alcohol (e.g., sucrose), and about 0.04% of a polysorbate (e.g., polysorbate 20), at pH 5.0 to 6.0. In certain embodiments, the pharmaceutical formulation comprises 100 to 200 mg/mL of the Factor XI and/or Factor XIa antibody, about 20 mM of histidine buffer, about 220 mM of a sugar or sugar alcohol (e.g., sucrose), and about 0.04% of a polysorbate (e.g., polysorbate 20), at pH 5.2 to 5.8. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of a sugar or sugar alcohol (e.g., sucrose), and about 0.04% of a polysorbate (e.g., polysorbate 20), at pH 5.45 to 5.55.

In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 0.10 to 0.30 mM of histidine buffer, 2.00 to 3.00 mM of a sugar or sugar alcohol (e.g., sucrose), and 0.0002% to 0.0006% of a polysorbate (e.g., polysorbate 20), at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.2 to 5.8. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of a sugar or sugar alcohol (e.g., sucrose), and about 0.0004% of a polysorbate (e.g., polysorbate 20), at pH 5.45 to 5.55.

In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 10 to 30 mM of histidine buffer, 200 to 300 mM of sucrose, and 0.02% to 0.06% of polysorbate 20, at pH 5.0 to 6.0. In certain embodiments, the pharmaceutical formulation comprises 100 to 200 mg/mL of the Factor XI and/or Factor XIa antibody, about 20 mM of histidine buffer, about 220 mM of sucrose, and about 0.04% of polysorbate 20, at pH 5.0 to 6.0. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of sucrose, and about 0.04% of polysorbate 20, at pH 5.3 to 5.7. In certain embodiments, the pharmaceutical formulation comprises 100 to 200 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 20 mM of histidine buffer, about 220 mM of sucrose, and about 0.04% of polysorbate 20, at pH 5.45 to 5.55.

In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), 0.10 to 0.30 mM of histidine buffer, 2.00 to 3.00 mM of sucrose, and 0.0002% to 0.0006% of polysorbate 20, at pH 5.0 to 6.0. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of sucrose, and about 0.0004% of polysorbate 20, at pH 5.0 to 6.0. In certain embodiments, the pharmaceutical formulation comprises 1.00 to 2.00 mg/mL of the Factor XI and/or Factor XIa antibody, 20 mM of histidine buffer, about 2.20 mM of sucrose, and about 0.0004% of polysorbate 20, at pH 5.3 to 5.7. In certain embodiments, the pharmaceutical formulation comprises 1.00 to 2.00 mg/mL of the Factor XI and/or Factor XIa antibody (e.g., an antibody that has a heavy chain variable domain (VH) having an amino acid sequence of SEQ ID NOs: 9 or 29, and a light chain variable domain (VL) having an amino acid sequence of SEQ ID NOs: 19 or 39), about 0.20 mM of histidine buffer, about 2.20 mM of sucrose, and about 0.0004% of polysorbate 20, at pH 5.45 to 5.55.

In embodiments, the present disclosure provides that a pharmaceutical formulation comprising an antibody that binds FXI and/or FXIa protein, or the antigen-binding fragment thereof, is contained in a vial in which the formulation includes an overfill volume for complete withdrawal of a therapeutically effective amount of the anti-FXI and/or anti-FXIa antibody or the antigen-binding fragment thereof. In certain embodiments, the vial contains a pharmaceutical formulation comprising about 150 mg of an antibody that binds FXI and/or FXIa protein (e.g., human, rabbit, cynomolgus monkey, and baboon FXI and/or FXIa), which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 20 mM; sucrose at a concentration of about 220 mM; and polysorbate-20 at a concentration of about 0.04% (v/v); and the pH of the formulation is about pH 5.5.

In embodiments, the present disclosure provides an intravenous delivery pharmaceutical formulation comprising about 1.5 mg of an antibody that binds FXI and/or FXIa protein (e.g., human, rabbit, cynomolgus monkey, and baboon FXI and/or FXIa), or the antigen-binding fragment thereof, which antibody has a heavy chain variable domain (VH) having an amino acid sequence of SEQ ID NOs: 9 or 29, and a light chain variable domain (VL) having an amino acid sequence of SEQ ID NOs: 19 or 39; a histidine buffer at a concentration of about 0.20 mM; sucrose at a concentration of about 2.20 mM; a polysorbate-20 at a concentration of about 0.0004% (v/v), and a diluent (e.g., dextrose 5% in water (D5W)); and the pH of the formulations is about pH 5.5.

Stability of the Factor XI and/or Factor XIa Antibody

The pharmaceutical formulations of the present invention exhibit high levels of stability. A pharmaceutical formulation is stable when the Factor XI and/or Factor XIa antibody within the formulation retains an acceptable degree of physical property, chemical structure, and/or biological function after storage under defined conditions.

Exemplary methods to determine stability of the Factor XI and/or Factor XIa antibody in the pharmaceutical formulation are described in Example 1 of the present disclosure. Additionally, stability of the protein can be assessed by measuring the binding affinity of the Factor XI and/or Factor XIa antibody to its targets or the biological activity of the Factor XI and/or Factor XIa antibody in certain in vitro assays, such as the aPTT and FXI activity assays described in WO 2016/207858.

The pharmaceutical formulation can be prepared and stored as a liquid formulation. In certain embodiments, the pharmaceutical formulation is a liquid formulation for storage at 2-8° C. (e.g., 4° C.). In certain embodiments, the pharmaceutical formulation is a liquid formulation for storage at 4° C. and protected from light.

Stability studies have found Antibody 1 150 mg/mL concentrate for solution for injection to be compatible with its excipients and primary packaging materials. Antibody 1 150 mg/mL concentrate for injection is suitable for subcutaneous administration with disposable syringes, without dilution or with dilution in a carrier buffer, e.g., 5% dextrose (D5W). Concentrate for injection with commercially available disposable syringes has been demonstrated for a dose range from 0.5 mg/subject to 600 mg/subject. Materials found to be compatible with Antibody 1 comprise injection syringes composed of polypropylene or polycarbonate, and needles for injection composed of stainless steel. Compatibility of Antibody 1 concentrate for solution for injection has been demonstrated with 1 mL syringes for Antibody 1 concentrations from 0.5 mg/mL to 150 mg/mL. Compatibility of Antibody 1 concentrate for solution for injection has been demonstrated with 3 mL syringes filled up to approximately 2 mL for an Antibody 1 concentration of 150 mg/mL, covering in total a dose range from 0.5 mg up to 150 mg for the 1 mL syringe and a dose of about 300 mg for the 3 mL syringe (filled with approximately 2 mL) per injection.

### Dosage Forms

Prior to pharmaceutical use, the pharmaceutical formulation can be diluted in an aqueous carrier if suitable for the route of administration. For intravenous administration, suitable carriers include sterile water for injection (SWFI), bacteriostatic water for injection (BWFI), a pH buffered solution (e.g., phosphate-buffered saline), sterile saline solution, Ringer's solution, or dextrose solution. For example, when the pharmaceutical formulation is prepared for intravenous administration, the pharmaceutical formulation comprises a 5% dextrose solution (D5W). In certain embodiments, the diluted pharmaceutical formulation is isotonic and suitable for administration by intravenous infusion, e.g., D5W. In certain embodiments, the formulation is diluted in about 50 mL D5W, 100 mL D5W, 150 mL D5W, 200 mL D5W, 250 mL D5W, 300 mL D5W, 350 mL D5W, 400 mL D5W, 450 mL D5W, 500 mL D5W, or 1 L D5W.

The pharmaceutical formulation comprises the Factor XI and/or Factor XIa antibody at a concentration suitable for storage. In certain embodiments, the pharmaceutical formulation comprises the Factor XI and/or Factor XIa antibody at a concentration of 100-200 mg/mL, 100-190 mg/mL, 100-180 mg/mL, 100-170 mg/mL, 100-160 mg/mL, 110-150 mg/mL, 120-150 mg/mL, 130-150 mg/mL, 140-150 mg/mL, 140-160 mg/mL, 140-170 mg/mL, 140-180 mg/mL, 140-190 mg/mL, 150-190 mg/mL, 150-180 mg/mL, 150-170 mg/mL, or 150-160 mg/mL. In certain embodiments, the pharmaceutical formulation comprises the Factor XI and/or Factor XIa antibody at a concentration of about 10 mg/mL, about 15 mg/mL, about 25 mg/mL, about 50 mg/mL, about 75 mg/mL, about 100 mg/mL, about 120 mg/mL, about 125 mg/mL, about 130 mg/mL, about 135 mg/mL, about 140 mg/mL, about 145 mg/mL, about 150 mg/mL, about 155 mg/mL, about 160 mg/mL, about 165 mg/mL, about 170 mg/mL, about 175 mg/mL, about 180 mg/mL, about 185 mg/mL, about 190 mg/mL, about 195 mg/mL, or about 200 mg/mL.

The pharmaceutical formulation comprises the Factor XI and/or Factor XIa antibody at a concentration suitable for storage. In certain embodiments, the pharmaceutical formulation comprises the Factor XI and/or Factor XIa antibody at a concentration of 1.00-2.00 mg/mL, 1.00-1.90 mg/mL, 1.00-1.80 mg/mL, 1.00-1.70 mg/mL, 1.00-1.60 mg/mL, 1.10-1.50 mg/mL, 1.20-1.50 mg/mL, 1.30-1.50 mg/mL, 1.40-1.50 mg/mL, 1.40-1.60 mg/mL, 1.40-1.70 mg/mL, 1.40-1.80 mg/mL, 1.40-1.90 mg/mL, 1.50-1.90 mg/mL, 1.50-1.80 mg/mL, 1.50-1.70 mg/mL, or 1.50-1.60 mg/mL. In certain embodiments, the pharmaceutical formulation comprises the Factor XI and/or Factor XIa antibody at a concentration of about 0.10 mg/mL, about 0.15 mg/mL, about 0.25 mg/mL, about 0.50 mg/mL, about 0.75 mg/mL, about 1.00 mg/mL, about 1.20 mg/mL, about 1.25 mg/mL, about 1.30 mg/mL, about 1.35 mg/mL, about 1.40 mg/mL, about 1.45 mg/mL, about 1.50 mg/mL, about 1.55 mg/mL, about 1.60 mg/mL, about 1.65 mg/mL, about 1.70 mg/mL, about 1.75 mg/mL, about 1.80 mg/mL, about 1.85 mg/mL, about 1.90 mg/mL, about 1.95 mg/mL, or about 2.00 mg/mL.

In certain embodiments, the pharmaceutical formulation is packaged in a vial (e.g., a vial, bag, pen, or syringe). In certain embodiments, the vial comprises an overfill to allow for complete removal of the intended dose. In certain embodiments, the vial comprises an overfill of 5 to 35%, 10 to 30%, 15 to 25%, or 10 to 20%. In a particular embodiment, the vial comprises an overfill of about 20%.

In certain embodiments, the formulation may be a liquid formulation. In certain embodiments, the amount of Factor XI and/or Factor XIa antibody in the container is suitable for administration as a single dose. In certain embodiments, the amount of Factor XI and/or Factor XIa antibody in the container is suitable for administration in multiple doses. In certain embodiments, the pharmaceutical formulation comprises the Factor XI and/or Factor XIa antibody at an amount of 0.1 to 200 mg. In certain embodiments, the pharmaceutical formulation comprises the Factor XI and/or Factor XIa antibody at an amount of 1 to 200 mg, 10 to 200 mg, 20 to 200 mg, 50 to 200 mg, 100 to 200 mg, 200 to 200 mg, 500 to 2000 mg, 1000 to 2000 mg, 0.1 to 1000 mg, 1 to 1000 mg, 10 to 1000 mg, 20 to 1000 mg, 50 to 1000 mg, 100 to 1000 mg, 200 to 1000 mg, 500 to 1000 mg, 0.1 to 500 mg, 1 to 500 mg, 10 to 500 mg, 20 to 500 mg, 50 to 500 mg, 100 to 500 mg, 200 to 500 mg, 0.1 to 200 mg, 1 to 200 mg, 10 to 200 mg, 20 to 200 mg, 50 to 200 mg, 100 to 200 mg, 0.1 to 100 mg, 1 to 100 mg, 10 to 100 mg, 20 to 100 mg, 50 to 100 mg, 0.1 to 50 mg, 1 to 50 mg, 10 to 50 mg, 20 to 50 mg, 0.1 to 20 mg, 1 to 20 mg, 10 to 20 mg, 0.1 to 10 mg, 1 to 10 mg, or 0.1 to 1 mg. In certain embodiments, the pharmaceutical formulation comprises the Factor XI and/or Factor XIa antibody at an amount of about 0.1 mg, about 0.5 mg, about 1 mg, about 1.5 mg, about 2 mg, about 2.5 mg, about 5 mg, about 10 mg, about 20 mg, about 30 mg, about 40 mg, about 50 mg, about 60 mg, about 70 mg, about 80 mg, about 90 mg, about 100 mg, about 150 mg, about 200 mg, about 250 mg, about 300 mg, about 400 mg, about 450 mg, about 500 mg, about 600 mg, about 700 mg, about 800 mg, about 900 mg, about 1000 mg, about 1500 mg, or about 2000 mg in the therapeutically effective amount.

### Dosage Regimens and Therapeutic Uses

In another aspect, the present disclosure provides a method for treating cancer, the method comprising administering to a subject in need thereof a Factor XI and/or Factor XIa antibody disclosed herein (e.g., Antibody 1) once a month.

In certain embodiments, the method further comprises administering to the subject, after the initial treatment cycle, the Factor XI and/or Factor XIa antibody in one or more monthly treatment cycles, e.g., for a period of 3-months, wherein the Factor XI and/or Factor XIa antibody is administered on Day 1, Day 31, and Day 61. The subsequent treatment cycles, in which the subject receives administration of the Factor XI and/or Factor XIa antibody once month, are designed to maintain a certain level of the Factor XI and/or Factor XIa antibody in the subject. In certain embodiments, the subject receives at least 1, 2, 3, 4, 5, 6, 7, 8, 9, 10, 11, 12, 13, 14, or 15 subsequent treatment cycles. In some embodiments, the subject remains on the treatment for life.

In some embodiments, the subject afflicted with or at risk of developing a thromboembolic disorder and who is undergoing a surgical procedure is administered the intravenous drug delivery formulation on the same day as the surgical procedure. In some embodiments, the intravenous drug delivery formulation is administered between 2 to 10 hours after surgery. In some embodiments, the intravenous drug delivery formulation is administered between 4 to 8 hours after surgery. In some embodiments, the intravenous drug delivery formulation is administered about 1 hour, about 2 hours, about 3 hours, about 4 hours, about 5 hours, about 6 hours, about 7 hours, about 8 hours, about 9 hours, or about 10 hours after surgery.

In certain embodiments, the one or more doses in the initial and subsequent treatment cycles comprise the Factor XI and/or Factor XIa antibody administered subcutaneously at a dose about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the one or more doses in the initial and subsequent treatment cycles comprise the Factor XI and/or Factor XIa antibody (e.g., Antibody 1) are administered subcutaneously at a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 185 mg, about 190 mg, about 195 mg, or about 200 mg. In some embodiments, the Factor XI and/or Factor XIa antibody is administered subcutaneously at a dose of about 90 mg. In some embodiments, the Factor XI and/or Factor XIa antibody is administered subcutaneously at a dose of about 120 mg. In some embodiments, the Factor XI and/or Factor XIa antibody is administered subcutaneously at a dose of about 150 mg. In some embodiments, the Factor XI and/or Factor XIa antibody is administered subcutaneously at a dose of about 180 mg. In any of the above embodiments, the Factor XI and/or Factor XIa antibody is administered subcutaneously monthly.

In some embodiments, the therapeutically effective dose range for the Factor XI and/or Factor XIa antibody (e.g., Antibody 1) following subcutaneous administration is about 75 mg to about 165 mg, about 80 mg to about 160 mg, about 85 mg to about 155 mg, or about 90 mg to about 160 mg. In certain embodiments, the therapeutically effective dose range for the Factor XI and/or Factor XIa antibody (e.g., Antibody 1) following subcutaneous administration is about 90 mg to about 160 mg.

In certain embodiments, the one or more doses in the initial and subsequent treatment cycles comprise the Factor XI and/or Factor XIa antibody (e.g., Antibody 1) are administered intravenously at a dose about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the one or more doses in the initial and subsequent treatment cycles comprise the Factor XI and/or Factor XIa antibody (e.g., Antibody 1) are administered intravenously at a dose of about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 100 mg, about 110 mg, about 120 mg, about 130 mg, about 140 mg, about 150 mg, about 160 mg, about 170 mg, about 180 mg, about 185 mg, about 190 mg, about 195 mg, or about 200 mg. In some embodiments, the Factor XI and/or Factor XIa antibody is administered intravenously at a dose of about 30 mg. In some embodiments, the Factor XI and/or Factor XIa antibody is administered intravenously at a dose of about 60 mg. In some embodiments, the Factor XI and/or Factor XIa antibody is administered intravenously at a dose of about 75 mg. In some embodiments, the Factor XI and/or Factor XIa antibody is administered intravenously at a dose of about 150 mg. In some embodiments, the Factor XI and/or Factor XIa antibody is administered intravenously in a single dose (e.g., to a subject undergoing a medical surgery, e.g., to a subject undergoing unilateral total knee arthroplasty (TKA), e.g., on the same day as surgery).

A physician can start doses of the antibodies of the present disclosure (e.g., Antibody 1) employed in the pharmaceutical composition at levels lower than that required to achieve the desired therapeutic effect and gradually increase the dosage until the desired effect is achieved. In general, effective doses of the compositions of the present disclosure, for the treatment of thromboembolic disorders described herein vary depending upon many different factors, including means of administration, target site, physiological state of the patient, other medications administered, and whether treatment is prophylactic or therapeutic. Treatment dosages may be titrated to optimize safety and efficacy. For systemic administration with an antibody, the dosage ranges from about 0.01 to 15 mg/kg of the host body weight. For administration (e.g., subcutaneous or intravenous administration) with an antibody, the dosage may range from 0.1 mg to 5 mg or from 1 mg to 600 mg. For example, an anti-FXI/FXIa antibody described herein (e.g., Antibody 1) can be administered at a dose of about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 2.1 mg/kg, about 2.2 mg/kg, about 2.3 mg/kg, about 2.4 mg/kg, about 2.5 mg/kg, about 2.6 mg/kg, about 2.7 mg/kg, about 2.8 mg/kg, about 2.9 mg/kg, about 3.0 mg/kg, about 3.1 mg/kg, about 3.2 mg/kg, about 3.3 mg/kg, about 3.4 mg/kg, about 3.5 mg/kg, about 3.6 mg/kg, about 3.7 mg/kg, about 3.8 mg/kg, about 3.9 mg/kg, about 4.0 mg/kg, about 4.1 mg/kg, about 4.2 mg/kg, about 4.3 mg/kg, about 4.4 mg/kg, about 4.5 mg/kg, about 4.6 mg/kg, about 4.7 mg/kg, about 4.8 mg/kg, about 4.9 mg/kg, or about 5.0 mg/kg.

In certain embodiments, the Factor XI and/or Factor XIa antibody is administered intravenously. For example, in certain embodiments, the Factor XI and/or Factor XIa antibody is administered by intravenous infusion, e.g., with a prefilled bag, a prefilled pen, or a prefilled syringe. In certain embodiments, the Factor XI and/or Factor XIa antibody, in a pharmaceutical formulation disclosed herein, is diluted prior to administration. For example, in certain embodiments, the pharmaceutical formulation is diluted with dextrose 5% in water (D5W) and is administered intravenously from a bag. The intravenous infusion may be for about one hour (e.g., 50 to 80 minutes). In certain embodiments, the bag is connected to a channel comprising a tube and/or a needle.

The types of thromboembolic disorders that can be treated with the Factor XI and/or Factor XIa antibody or pharmaceutical formulation disclosed herein include but are not limited to A “thromboembolic,” or similar terms as used herein, can also refer to any number of the following, which the anti-FXI and/or FXIa antibodies or antigen binding fragments thereof of the present disclosure can be used to prevent or treat: thromboembolism in subjects with suspected or confirmed cardiac arrhythmia such as paroxysmal, persistent or permanent atrial fibrillation or atrial flutter; stroke prevention in atrial fibrillation (SPAF), a subpopulation of which is AF patients undergoing percutaneous coronary interventions (PCI); acute venous thromboembolic events (VTE) treatment and extended secondary VTE prevention in patients at high risk for bleeding; cerebral and cardiovascular events in secondary prevention after transient ischemic attack (TIA) or non-disabling stroke and prevention of thromboembolic events in heart failure with sinus rhythm; venous thromboembolism in pediatric subjects (pediatric VTE); clot formation in left atrium and thromboembolism in subjects undergoing cardioversion for cardiac arrhythmia; thrombosis before, during and after ablation procedure for cardiac arrhythmia; venous thrombosis, this includes but not exclusively, treatment and secondary prevention of deep or superficial veins thrombosis in the lower members or upper member, thrombosis in the abdominal and thoracic veins, sinus thrombosis and thrombosis of jugular veins; thrombosis on any artificial surface in the veins or arteries like catheter, pacemaker wires, synthetic arterial grafts; mechanical or biological heart valves or left ventricular assist device; pulmonary embolism in patients with or without venous thrombosis; Chronic Thromboembolic Pulmonary Hypertension (CTEPH); arterial thrombosis on ruptured atherosclerotic plaque, thrombosis on intra-arterial prosthesis or catheter and thrombosis in apparently normal arteries, this includes but not limited to acute coronary syndromes, ST elevation myocardial infarction, non ST elevation myocardial infarction, unstable angina, stent thrombosis, thrombosis of any artificial surface in the arterial system and thrombosis of pulmonary arteries in subjects with or without pulmonary hypertension; thrombosis and thromboembolism in patients undergoing percutaneous coronary interventions (PCI); cardioembolic and cryptogenic strokes; non-central nervous systemic embolism (non-CNS systemic embolism); hemorrhagic stroke; thrombosis in patients with invasive and non-invasive cancer malignancies; thrombosis over an indwelling catheter; thrombosis and thromboembolism in severely ill patients; cardiac thrombosis and thromboembolism, this includes but not exclusively cardiac thrombosis after myocardial infarction, cardiac thrombosis related to condition such as cardiac aneurysm, myocardial fibrosis, cardiac enlargement and insufficiency, myocarditis and artificial surface in the heart; thromboembolism in patients with valvular heart disease with or without atrial fibrillation; thromboembolism over valvular mechanic or biologic prostheses; thromboembolism in patients who had native or artificial cardiac patches, arterial or venous conduit tubes after heart repair of simple or complex cardiac malformations; venous thrombosis and thromboembolism after knee replacement surgery, hip replacement surgery, and orthopedic surgery, thoracic or abdominal surgery; arterial or venous thrombosis after neurosurgery including intracranial and spinal cord interventions; congenital or acquired thrombophilia including but not exclusively factor V Leiden, prothrombin mutation, antithrombin III, protein C and protein S deficiencies, factor XIII mutation, familial dysfibrinogenemia, congenital deficiency of plasminogen, increased levels of factor XI, sickle cell disease, antiphospholipid syndrome, autoimmune disease, chronic bowel disease, nephrotic syndrome, hemolytic uremia, myeloproliferative disease, disseminated intra vascular coagulation, paroxysmal nocturnal hemoglobinuria and heparin induced thrombopenia; thrombosis and thromboembolism in chronic kidney disease; and thrombosis and thromboembolism in patients undergoing hemodialysis and in patients undergoing extra-corporal membrane oxygenation. In certain embodiments, the subject treated with the Factor XI and/or Factor XIa antibody or pharmaceutical formulation disclosed herein is obese (e.g., severely obese, e.g., with body-mass index (BMI)≥35 kg/m²). In certain embodiments, the subject treated with the Factor XI and/or Factor XIa antibody or pharmaceutical formulation disclosed herein is not obese. In certain embodiments, the obese subject is associated with lower exposure following administration of the same dose of the Factor XI and/or Factor XIa antibody (e.g., Antibody 1), as the non-obese subject. In certain embodiments, the exposure is about 10%, about 20%, about 30%, about 40%, about 50%, about 60%, about 70%, about 80%, or about 90% less for the obese subject following administration of the same dose of the Factor XI and/or Factor XIa antibody (e.g., Antibody 1), as the non-obese subject. In certain embodiments, the obese subject is associated with shorter duration of aPTT prolongation following administration of the same dose of the Factor XI and/or Factor XIa antibody (e.g., Antibody 1), as the non-obese subject. In certain embodiments, the aPTT prolongation is about 10%, about 20%, about 30%, about 40%, about 50%, about 60%, about 70%, about 80%, or about 90% shorter for the obese subject following administration of the same dose of the Factor XI and/or Factor XIa antibody (e.g., Antibody 1), as the non-obese subject.

The CHA2DS2-VASc risk score is a validated and widely used stratification tool to predict thromboembolic risk in AF patients and to identify patients who should benefit from anticoagulation therapy (LIP 2011; Camm, et al. (2012) Eur Heart J 2012; 33: 2719-2747); the accumulated evidence shows that CHA2DS2-VASc is at least as accurate as or possibly better than, scores such as CHADS2 in identifying patients who develop stroke and thromboembolism and definitively better at identifying ‘truly low-risk’ patients with AF. The CHA2DS2-VASc risk score ranges from 0 to a maximum score of 9. In certain embodiments, the subject treated with the Factor XI and/or Factor XIa antibody or pharmaceutical formulation disclosed herein has a CHA2DS2-VASc risk score of 0-1 for men and 1-2 for women. In certain embodiments, the subject treated with the Factor XI and/or Factor XIa antibody or pharmaceutical formulation disclosed herein has a CHA2DS2-VASc risk score ≥2 for men and >3 for women. In certain embodiments, the subject treated with the Factor XI and/or Factor XIa antibody or pharmaceutical formulation disclosed herein has a CHA2DS2-VASc risk score ≥4 or ≥3 with at least 1 of planned concomitant use of anti-platelet medication (e.g., aspirin and/or P2Y12 inhibitor) or CrCl≤50 ml/min by the Cockcroft-Gault equation.

The Factor XI and/or Factor XIa antibody disclosed herein (e.g., Antibody 1) can be used as a monotherapy or in combination with one or more therapies. Such combination therapies may be useful for treating thromboembolic disorders, such as, ischemic stroke (cardioembolic, thrombotic) or systemic embolism, AF, stroke prevention in AF (SPAF), deep vein thrombosis, venous thromboembolism, pulmonary embolism, acute coronary syndromes (ACS), acute limb ischemia, chronic thromboembolic pulmonary hypertension, or systemic embolism). In certain embodiments, the Factor XI and/or Factor XIa antibody is used as a monotherapy in accordance with the dosage regimen disclosed herein. In other embodiments, the Factor XI and/or Factor XIa antibody is used in combination with one or more therapies, wherein the Factor XI and/or Factor XIa antibody is administered in accordance with the dosage regimen disclosed herein and the one or more therapies are administered in accordance with a dosage regimen known to be suitable for treating the particular subject with the particular disorder.

In some aspects, statin therapies may be used in combination with the FXI/FXIa antibodies and antigen binding fragments, or formulations comprising said FXI/FXIa antibodies and antigen binding fragments (e.g., Antibody 1), described in the present disclosure for the treatment of patients with thrombotic and/or thromboembolic disorders. In particular aspects, non-limiting examples of therapeutic active agents suitable for use in combination with an anti-FXI/FXIa antibody described herein (e.g., Antibody 1) include thromboxane inhibitors (e.g., aspirin), adenosine diphosphate receptor antagonists (or P2Y12 inhibitors) such as thienopyridines (e.g., clopidogrel and prasugrel) and nonthienopyridines (e.g., ticagrelor and cangrelor), protease-activated receptor-1 (PAR1) antagonists (e.g., vorapaxar and atopaxar), and proton pump inhibitors (PPIs) (e.g., omeprazole, diazepam, phenytoin, lansoprazole, dexlansoprazole, rabeprazole, pantoprazole, esomeprazole, and naproxen). The use of PPIs in combination therapy may be suitable in cases where a subject has or has a history of a GI disorder, such as previous GI bleed or antecedent of peptic ulcer. In one aspect, the subject is being treated with non-steroidal anti-inflammatory drugs (NSAIDs), and is administered an anti-FXI/FXIa antibody described herein (e.g., Antibody 1) in combination with a proton pump inhibitor (e.g., omeprazole, diazepam, phenytoin, lansoprazole, dexlansoprazole, rabeprazole, pantoprazole, esomeprazole, and naproxen). In certain embodiments, a subject treated with the FXI/FXIa antibodies and antigen binding fragments, or formulations comprising said FXI/FXIa antibodies and antigen binding fragments (e.g., Antibody 1), are administered a direct oral anticoagulant (DOAC) following the duration of treatment (e.g., on the same day as end of treatment). In certain embodiments, a subject treated with the FXI/FXIa antibodies and antigen binding fragments, or formulations comprising said FXI/FXIa antibodies and antigen binding fragments (e.g., Antibody 1), are administered a Vitamin K Antagonist (VKA) following the duration of treatment (e.g., about 5 days before end of treatment, or about 3 days before end of treatment).

In certain embodiments, the method of treatment disclosed herein results in a disease response or improved survival of the subject or patient. For example, in certain embodiments, the disease response is a complete response, a partial response, or a stable disease. In certain embodiments, the improved survival is improved progression-free survival (PFS) or overall survival. Improvement (e.g., in PFS) can be determined relative to a period prior to initiation of the treatment of the present disclosure. Methods of determining disease response (e.g., complete response, partial response, or stable disease) and patient survival (e.g., PFS, overall survival) for BTC (e.g., advanced BTC, metastatic BTC), or biliary tract tumor therapy, are routine in the art and are contemplated herein. In some embodiments, disease response is evaluated according to RECIST 1.1 after subjecting the treated patient to contrast-enhanced computed tomography (CT) or magnetic resonance imaging (MM) of the affected area (e.g., chest/abdomen and pelvis covering the area from the superior extent of the thoracic inlet to the symphysis pubis).

### Examples

The disclosure now being generally described, will be more readily understood by reference to the following examples, which are included merely for purposes of illustration of certain aspects and embodiments of the present disclosure, and are not intended to limit the scope of the disclosure in any way.

### Example 1: Formulation, Packaging, and Storage of Antibody 1

### Study Design

The study was comprised of the following experimental parts:
Substage A: Compatibility feasibility testing—
Assess quantification limits for SE-HPLC assay for appropriate quantification of the diluted drug product in 5% dextrose (include linearity, reproducibility, LoQ and autosampler stability).
Substage B: Simulated administration compatibility study
Bag administration
Part 1: Short-term in-use stability in three (3) IV infusion bag administration system after adding drug product (low, intermediate and high dose) to the infusion bag. The drug product was diluted with 5% dextrose to obtain the target concentration, the dilution will be performed directly in the infusion bag (high dose), or via a pre-dilution (intermediate dose, low dose).
Part 2: Simulated intravenous (IV) administration testing was performed with commercially available administration materials for clinical use. Infusion bags in (3) materials (PE, PVC, PP) and infusion lines of two (2) material (PE, PVC), plus two (2) in-line filter and catheter were tested using a bracketing design with low, intermediate and high dose concentration levels.
Drug product stability and recovery from the infusion system was determined.

### Materials and Methods

### Test Material

The active drug product (DP), Antibody 1 150 mg/1 mL concentrate for solution for injection was presented in the following formulation: 20 mM L-histidine/histidine hydrochloride (histidine buffer) at pH 5.5, 220 mM sucrose, 0.04% (w/v) polysorbate 20, pH 5.5, at a nominal concentration of 150 mg/ml. The nominal DP concentration of 150 mg/ml was used for all calculations. The nominal fill volume was 1 mL in 6R glass vials (Type I).

### Analytical Methods

All equipment utilized for the analytical methods was documented in the study data file using the iLAB and Laboratory Information and Management System (LIMS) by the unique identification number or serial number, with the current version of software identified as appropriate. All equipment used was appropriately qualified and calibrated. If not stated otherwise, one sample was analysed or one injection per sample was performed for each time point of testing. Test methods utilized are summarized in Table 2.

TABLE 2
Analytical Test Methods

| Number | Test method |
|---|---|
| 1 | Test method for visible particles (PhEur 2.9.20) |
| 2 | Clarity/opalescence of solution |
| 3 | Determination of color using the LICO 690 Colorimeter |
| 4 | Sub-visible particles by LO according to USP <787> (small |
| | volume) |
| 5 | pH |
| 6 | Density |
| 7 | Assay by SEC-HPLC |
| 8 | Purity by SEC-HPLC |

### Calculation of Recovery (%)

For the low, intermediate, and high doses, the recovery was calculated using the peak area and concentration according to the formulae in Table 3.

TABLE 3
Recovery calculation formulae

| Calculation | Formula |
|---|---|
| Calculation of the concentration by SE-HPLC | $\begin{matrix}Concentration = \\ Dilution ( mg / mL )\end{matrix} \begin{matrix}( ( Peak area of the test sample ) \times ) \times \\ \frac{( Concentration of the control DP mg / mL )}{( Peak area of the control DP in respective diluent )}\end{matrix}$ |
| Recovery from infusion bags | $\begin{matrix}Recovery = \\ 100 ( \% )\end{matrix} \frac{( Concentration of the test sample ) \times }{( Theoretical concentration of the solution for dosing * )}$ |

*determined by weight and nominal concentration of DP, 150 mg/mL

### Experimental Setup

### Analytical Assessment of SE-HPLC

Depending on the recommended dosage levels and diluents, the drug product was serially diluted to a concentration range in diverse diluents used for compatibility testing. Table 4 illustrates dilution concentrations for the study.

TABLE 4
Target concentrations for the standard curve
for SEC assessment

| | Dilutions |
|---|---|
| Type of diluent | [mg/mL] |
| Dextrose 5% | ^{a}0.48 |
| | ^{a}0.75 |
| | 0.20 |
| | 0.30 |
| | 0.50 |
| | ^{c}0.75 |
| | 0.90 |
| SEC Mobile phase/^{b}Apn 1/1 (v/v) | ^{a}0.48 |
| (additional as control) | ^{a}0.75 |
| | 0.20 |
| | 0.30 |
| | 0.50 |
| | ^{c}0.75 |
| | 0.90 |

^{a}6 preparations and injected once from each preparation (repeatability assessment). Reinjection after 24 hours of the first repeatability sample to assess autosampler stability.
^{b}Apn Aprotinin,
^{c}test concentration in test method

### Concentration Bracketing

The concentration bracketing range was defined, based on the dosing provided by the Customer, with 0.50 mg/mL as minimum concentration (low dose) and 3.0 mg/mL as maximum concentration (high dose) (Table 5). An intermediate dose was assessed at 1.5 mg/mL. Dosing solutions were prepared as described in Table 5 and Table 7.

TABLE 5
Concentration bracketing for infusion setup substage B

| | Target cone. | Testing conc. | Flow rate |
|---|---|---|---|
| Dose | [mg/mL] | [mg/mL] | [mL/Hour] |
| Low dose (LD) | 0.50 | 0.48^{a} | ^{d}P: 100 |
| | | | ^{e}I: 60 |
| Intermediate dose | 1.5 | 1.5 ^{b} | ^{d}P: 100 |
| (ID) | | | ^{e}I: 100 |
| High dose (HD) | 3.0 | 3.15 ^{c} | ^{d}P: 100 |
| | | | ^{e}I: 100 |

^{a}95% of target concentration,
^{b} 100% of target concentration,
^{c} 105% of target concentration,
^{d}P: Priming,
^{e}I: Infusion

### Preparation of Dosing Solutions

An appropriate number of DP vials were removed from refrigerated storage and equilibrated at ambient temperature until no longer cold to the touch (should not be longer than 1 hour). Pre-dilutions and samples for substage A were prepared by pipetting according to Table 6.

For substage B, the simulated clinical administration compatibility testing, IV infusion bags were prepared according to Table 7 using disposable syringes of appropriate size. Pre-dilutions were prepared in PETG bottles. The final dosing solutions were prepared directly in the infusion bags.

TABLE 6
Dilution scheme for preparation of sample solutions for substage A -
SE-HPLC assessment

| Dilution Step 1 | | | | Dilution Step 2 | | | |
|---|---|---|---|---|---|---|---|
| V_{DP} | V_{Dil} | V_{Total} | Concentration | V_{DP} | V_{Dil} | V_{Total} | Concentration |
| [mL] | [mL] | [mL] | D1 [mg/mL] | [mL] | [mL] | [mL] | D1 [mg/mL] |
| 0.020 | 0.280 | 0.300 | 10 | 0.020 | 0.980 | 1.0 | 0.20 |
| | | | | 0.030 | 0.970 | 1.0 | 0.30 |
| | | | | 0.050 | 0.950 | 1.0 | 0.50 |
| | | | | 0.075 | 0.925 | 1.0 | 0.75* |
| | | | | 0.090 | 0.910 | 1.0 | 0.90 |
| | | | | 0.048 | 0.952 | 1.0 | 0.48* |

V_{DP} - volume DP,
V_{Dil} - volume diluent,
V_{Total} - volume Dilution 1
*Prepared six times independently for repeatability asssessment

For the low dose, DP was diluted in dextrose 5% to 15 mg/mL in a suitable container. 3.2 mL of diluent were removed from the bag then 3.2 mL of pre-diluted DP were injected using a B. Braun Omnifix® 5 mL syringe via a 21G SS needle. The bag with the injection solution was mixed.

For the intermediate dose, DP was diluted in dextrose 5% to 15 mg/mL in a suitable container. 10.0 mL of diluent were removed from the bag then 10.0 mL of pre-diluted DP were injected using a 10 mL BD Plastipak syringe via a 21G SS needle. The bag with the injection solution was mixed.

For the high dose, 2.1 mL of diluent were removed from the bag then 2.1 mL of undiluted DP were injected using a 3 mL BD Plastipak syringe via a 21G SS needle. The bag with the injection solution was mixed.

TABLE 7
Dilution scheme for preparation of dosing solutions for substage B

| | | | | | | | Injection into the | | |
|---|---|---|---|---|---|---|---|---|---|
| | Dose | | Dilution Step 1 | | | | IV bag | | |
| | | Target | | | | Concentration | | | |
| | | conc. | V_{DP} | V_{Dil} | V_{Total} | D1 | V_{D1} | V_{Dil} | V_{Total} |
| | Level | [mg/mL] | [mL] | [mL] | [mL] | [mg/mL] | [mL] | [mL] | [mL] |
| Setup - Bag | Low | 0.48 | 1.5 | 13.5 | 15.0 | 15.0 | 3.2 | 96.8^{a} | 100 |
| Adminstration | dose | | | | | | | | |
| | Intermediate | 1.5 | 3.5 | 31.5 | 35.0 | 15.0 | 10.0 | 90.02 | 100 |
| | dose | | | | | | | | |
| | High | 3.15 | N/A | N/A | N/A | 150.0^{b} | 2.1 | 97.9^{a} | 100 |
| | dose | | | | | | (V_{DP}) | | |

N/A - not applicable;
V_{DP} - volume DP,
V_{Dil} - volume diluent,
V_{Total} - volume Dilution 1
^{a}Calculated as (Theoretical Volume of the bag − Volume of diluent withdrawn (= Vol of D1 injected)),
^{b}undiluted DP

### Substage A: SE-HPLC Assessment

### SE-HPLC Assessment

The prepared samples (according to Table 6) were used to assess linearity, repeatability, limit of quantification and autosampler stability.

### Substage B: Simulated Administration Compatibility Study

### In-Use Stability

The prepared infusion bags (according to Table 7) were incubated:
For 3 hours at room temperature with ambient room light exposure,
For 20 hours at 2-8° C.,
For 1 hour at room temperature with ambient room light exposure.

### Simulated Administration Testing

At the end of the storage time (a cumulative 24 hours), the infusion bags were used for the simulated administration testing.

The infusion line, extension set with in-line filter and catheter were connected to the infusion bag. The bags were prepared with solution at a target concentration as described in Table 5. The bag system was primed until the first drop at a flow rate of 100 mL per hour. After priming of the infusion systems (bag, line, inline-filter and catheter), the simulated administration was performed with the remaining solution in the infusion bag using the infusion pump at a flow rate as described in Table 5, infusion was performed until the infusion bag was empt. After administration, the in-line filter was disconnected to allow collection of a sample from the part of the line and bag immediately prior to the filter.

For LD samples, a partial infusion was performed using the infusion pump at a flow rate as described in Table 5 for a total of 60 mL. After administration, the remaining solution in the bag was collected separately, and the in-line filter was disconnected to allow collection of a sample from the part of the line and bag immediately prior to the filter.

The total contact time of the dosing solution with the infusion set was approximately 1 hour. At the end of the simulated administration, the cumulative contact time with the infusion material was approximately 25 hours. The cumulative time the dosing solution was under room temperature and ambient light conditions was approximately 5 hours. The simulated administration was performed under ambient temperature and light conditions in a biosafety cabinet (Class II).

### Simulated Administration Testing

Per combination and dose level, the following samples were collected in sterile PETG bottles and tested according to Table 8.
Sample 1 (T0): Initial solution (4 mL sample).
Sample 2 (T24): Aliquot from bag after incubation for 4 hours at ambient temperature and 20 hours at 2-8° C. (4 mL sample).
Sample 3 (Post-filter): Solution for infusion (after in-line filter) from the total administration (4 mL of sample from total administered volume).
Sample 4 (Pre-filter): Solution for infusion prior to in line filter (4 mL sample from line and bag).

TABLE 8
Analytical testing scheme for Stage B - Simulated Administration Compatibility testing

| | | | | Analytical assays | | | | | |
|---|---|---|---|---|---|---|---|---|---|
| | Testing | Vol. | | | | | | | SE-HPLC |
| | dose | approx. | Time | Visible | Clarity/ | | | Subvisible | Purity/ |
| Dose | [mg/mL] | (mL) | (h) | particles | opalescence | Color | pH | particles | Recovery |
| Low dose (LD) | 0.48 | 4 | 0 | X | X | X | X | X | X |
| | | | 24 | X | X | X | X | X | X |
| | | | Post | X | X | X | X | X | X |
| | | | filter | | | | | | |
| | | | Pre | X | X | X | X | X | X |
| | | | filter | | | | | | |
| Intermediate dose | 1.5 | 4 | 0 | X | X | X | X | X | X |
| (LD) | | | 24 | X | X | X | X | X | X |
| | | | Post | X | X | X | X | X | X |
| | | | filter | | | | | | |
| | | | Pre | X | X | X | X | X | X |
| | | | filter | | | | | | |
| High dose (HD) | 3.15 | 4 | 0 | X | X | X | X | X | X |
| | | | 24 | X | X | X | X | X | X |
| | | | Post | X | X | X | X | X | X |
| | | | filter | | | | | | |
| | | | Pre | X | X | X | X | X | X |
| | | | filter | | | | | | |

### Results and Discussion

Substage a— Analytical Assessment of the SE-HPLC Method

Based on the recommended dosage levels, the drug product was diluted to a concentration range from 0.20-0.90 mg/mL in dextrose (D5W) and an APN mixture containing mobile phase (MP) and aprotinin (APN) (MP:APN=1:1). Assessing concentrations above 0.90 mg/mL offered no an additional benefit, as samples are diluted to 0.75 mg/mL during SE-HPLC sample preparation.

### Linearity

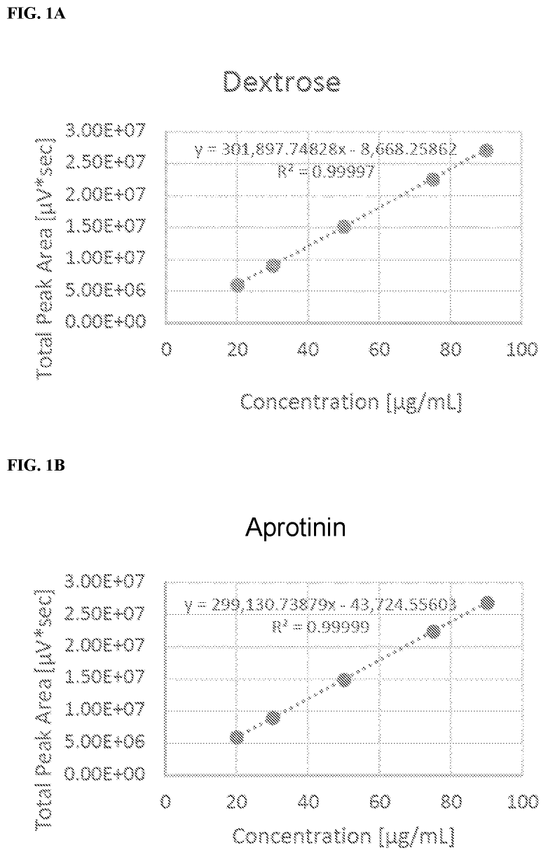

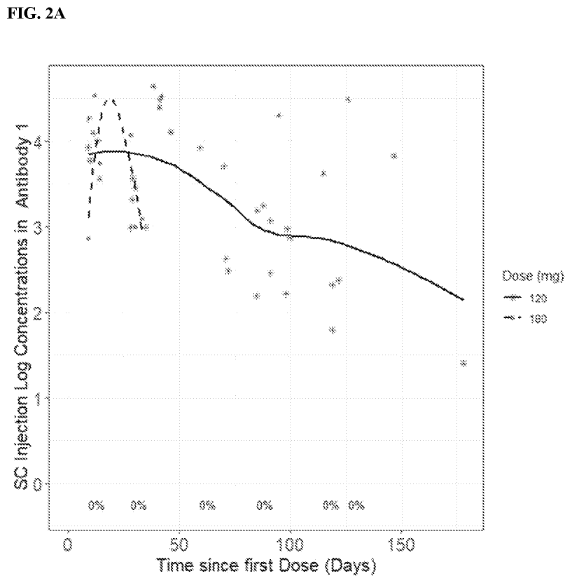

The dilution scheme as shown in Table 4 was performed with dextrose (D5W) or an APN mixture. The APN mixture served as a positive control by minimizing non-specific binding of product protein to contact surfaces. Linearity was evaluated. Linearity was established in dextrose in a range between 0.20-0.90 mg/mL (dextrose shown in FIG. 1A, APN shown in FIG. 1B). For subsequent studies, a one-point assessment was done to quantify the recovery against the standard in the respective diluent.

### Repeatability and Autosampler Stability

The drug product was diluted to 0.48 and 0.75 mg/mL, each concentration was prepared 6 times separately, and each preparation was injected once. The RSD of the repeatability assessment are shown in Table 9 (repeatability [RSD %]). The first preparation of each concentration of the repeatability assessment was reinjected after 24 hours standing at 5° C. in HPLC vials to assess autosampler stability, as shown in Table 9 (Absolute peak % difference [T24 compared to T0]).

TABLE 9
Analytical assessment of SE-HPLC method-repeatability
and autosampler stability

| | 0.48 mg/mL | | 0.75 mg/mL | |
|---|---|---|---|---|
| | | Absolute | | Absolute |
| | | peak % | | peak % |
| | | difference | | difference |
| | | (T24 | | (T24 |
| Diluent: | Repeatability | compared | Repeatability | compared |
| Dextrose 5% | (RSD %) | to T0) | (RSD %) | to T0) |
| % Main | 0.04 | 0.0039 | 0.03 | 0.0165 |
| % HMWS | 2.70 | 0.0142 | 1.39 | −0.003 |
| % LMWS | 8.67 | −0.0181 | 7.05 | −0.0135 |
| Total Peak | | 2.0 | | 0.3 |
| Area | | | | |

Based on the assessment, the SE-HPLC test method (CHVI-260130) was considered suitable for further studies. The sample concentration (mg/mL) was calculated from SE-HPLC data. For the high dose the DP was used as a reference (diluted to the final concentration as required by the test method), for the intermediate and low dose the intermediate dilution was used as a reference (diluted to the final concentration as required by the test method).

### LoQ Suitability Assessment

The drug product was diluted to 0.00075 mg/mL (0.1% of nominal target concentration). The sample obtained was injected once in the HPLC system and the relative S/N ratio calculated using Empower custom field “USP s/n”. The ratio obtained was 21.085, stating that the method is suitable for the determination of concentration values at the 0.1% of the nominal concentration from the test method.

### Substage B— Simulated Administration Compatibility Study

Table 10 and Table 11 summarize the results of the simulated administration compatibility testing.

At all dose levels, the initial, incubated and administered solutions of the bag administration setups showed no relevant changes in visible particles, colour, turbidity and purity by SE-HPLC. The pH values were between 4.5-5.9 for all dose levels and materials throughout the study. The pH shift can be explained by buffer dilution.

Subvisible particles were measured using the light obscuration method. The samples collected post-filter showed effective reduction in subvisible particle counts. For the sample post-filter of all combinations tested, subvisible particles by light obscuration were within the acceptance limits of USP <787> considering volumes below or equal to 100 mL, with cumulative subvisible particle counts ≥25 μm being less than or equal to 600 counts per container and cumulative subvisible particle counts ≥10 μm being less than or equal to 6000 counts per container.

The recovery was ≥93% for all samples (low dose, intermediate dose and high dose) in all the material combinations (PE, PVC, and PP).

TABLE 10
Summary of results for simulated administration assessment

| | | | | | | | | | HIAC: sub-visible particles | | | |
|---|---|---|---|---|---|---|---|---|---|---|---|---|
| | | Testing | | | | Clarity/ | | | (cumulative counts/mL) | | | |
| | Container | dose | Vol. | | Visible | opalescence | Color | | ≥2 | ≥5 | ≥10 | ≥25 |
| Setup | Type | (mg/mL) | (mL) | Time (h) | particies | (NTU) | (by series) | pH | μm | μm | μm | μm |
| Bag | PE Bag | 0.48 | 60 | 0 | PVFP | 0 | Colorless | 4.7 | 348 | 88 | 9 | 0 |
| | Ecoflac | | | 24 | PVFP | 0 | Colorless | 4.8 | 268 | 55 | 4 | 0 |
| | plus ® | | | Post filter | PVFP | 0 | ≥BY7 | 5.9 | 92 | 31 | 2 | 0 |
| | | | | Pre filter | PVFP | 0 | Colorless | 4.8 | 353 | 59 | 3 | 0 |
| | | 1.5 | 100 | 0 | PVFP | 0 | ≥BY7 | 5.2 | 530 | 182 | 25 | 0 |
| | | | | 24 | PVFP | 1 | Colorless | 5.2 | 221 | 41 | 12 | 8 |
| | | | | Post filter | PVFP | 0 | Colorless | 5.3 | 56 | 9 | 0 | 0 |
| | | | | Pre filter | PVFP | 0 | Colorless | 5.1 | 225 | 28 | 4 | 0 |
| | | 3.15 | 100 | 0 | PVFP | 0 | ≥BY7 | 5.3 | 7624 | 3582 | 723 | 0 |
| | | | | 24 | PVFP | 0 | Colorless | 5.4 | 225 | 59 | 8 | 0 |
| | | | | Post filter | PVFP | 0 | Colorless | 5.5 | 561 | 109 | 2 | 0 |
| | | | | Pre filter | PVFP | 1 | Colorless | 5.4 | 250 | 86 | 21 | 0 |
| Bag | PVC Bag | 0.48 | 60 | 0 | PVFP | 0 | Colorless | 4.7 | 991 | 148 | 7 | 0 |
| | Viaflex ® | | | 24 | PVFP | 0 | Colorless | 4.7 | 764 | 63 | 2 | 0 |
| | | | | Post filter | PVFP | 1 | Colorless | 5.1 | 234 | 73 | 28 | 1 |
| | | | | Pre filter | PVFP | 1 | Colorless | 4.8 | 1188 | 142 | 8 | 0 |
| | | 1.5 | 100 | 0 | PVFP | 0 | ≥BY7 | 5.1 | 1367 | 416 | 78 | 0 |
| | | | | 24 | PVFP | 1 | Colorless | 5.0 | 593 | 39 | 11 | 7 |
| | | | | Post filter | PVFP | 0 | Colorless | 5.2 | 23 | 3 | 1 | 0 |
| | | | | Pre filter | PVFP | 0 | Colorless | 5.1 | 473 | 36 | 4 | 0 |
| | | 3.15 | 100 | 0 | PVFP | 0 | ≥BY7 | 5.5 | 9788 | 2624 | 285 | 0 |
| | | | | 24 | PVFP | 0 | ≥BY7 | 5.3 | 649 | 41 | 0 | 0 |
| | | | | Post filter | PVFP | 0 | Colorless | 5.4 | 33 | 1 | 0 | 0 |
| | | | | Pre filter | PVFP | 0 | Colorless | 5.4 | 608 | 55 | 3 | 0 |
| Bag | PP Bag | 0.48 | 60 | 0 | PVFP | 0 | Colorless | 4.6 | 223 | 44 | 3 | 0 |
| | Ecoflac | | | 24 | PVFP | 0 | Colorless | 4.6 | 159 | 27 | 3 | 0 |
| | plus ® | | | Post filter | PVFP | 0 | Colorless | 5.0 | 23 | 3 | 3 | 0 |
| | | | | Pre filter | PVFP | 0 | Colorless | 4.6 | 331 | 93 | 13 | 0 |
| | | 1.5 | 100 | 0 | PVFP | 0 | ≥BY7 | 5.2 | 448 | 154 | 13 | 0 |
| | | | | 24 | PVFP | 1 | Colorless | 5.0 | 96 | 14 | 3 | 1 |
| | | | | Post filter | PVFP | 0 | Colorless | 5.2 | 40 | 9 | 3 | 0 |
| | | | | Pre filter | PVFP | 0 | Colorless | 5.0 | 303 | 77 | 24 | 3 |
| | | 3.15 | 100 | 0 | PVFP | 0 | Colorless | 5.2 | 3621 | 1260 | 166 | 0 |
| | | | | 24 | PVFP | 0 | Colorless | 5.3 | 144 | 23 | 1 | 0 |
| | | | | Post filter | PVFP | 0 | Colorless | 5.4 | 54 | 7 | 0 | 0 |
| | | | | Pre filter | PVFP | 1 | Colorless | 5.3 | 941 | 108 | 18 | 0 |

PVFP—practically free of visible particles (0 particles per sample),
BY—brown yellow

TABLE 11
Summary of SE-HPLC results for simulated administration assessment

| | | Testing | | | Protein | | Purity by SE-HPLC | | |
|---|---|---|---|---|---|---|---|---|---|
| | Container | dose | Vol. | | content | Recovery | Main | HMWS | LMWS |
| Setup | Type | (mg/mL) | (mL) | Time (h) | (mg/mL) | (%) | (%) | (%) | (%) |
| Bag | PP Bag | 0.48 | 60 | 0 | 0.45 | 102 | 98.4 | 1.5* | 0.2* |
| | Ecoflac | | | 24 | 0.45 | 101 | 98.4 | 1.5* | 0.2* |
| | plus ® | | | Post filter | 0.44 | 101 | 98.4 | 1.5* | 0.1* |
| | | | | Pre filter | 0.45 | 102 | 98.4 | 1.4* | 0.1* |
| | | 1.5 | 100 | 0 | 1.28 | 96 | 98.4 | 1.4 | 0.2 |
| | | | | 24 | 1.29 | 96 | 98.4 | 1.4 | 0.2 |
| | | | | Post filter | 1.28 | 95 | 98.3 | 1.5 | 0.2 |
| | | | | Pre filter | 1.30 | 97 | 98.3 | 1.5 | 0.2 |
| | | 3.15 | 100 | 0 | 2.76 | 96 | 98.3 | 1.5 | 0.2 |
| | | | | 24 | 2.77 | 96 | 98.3 | 1.5 | 0.2 |
| | | | | Post filter | 2.68 | 93 | 98.4 | 1.4 | 0.2 |
| | | | | Pre filter | 2.74 | 95 | 98.3 | 1.5 | 0.2 |
| Bag | PVC Bag | 0.48 | 60 | 0 | 0.44 | 102 | 98.3 | 1.5* | 0.2* |
| | Viaflex ® | | | 24 | 0.44 | 102 | 98.3 | 1.5* | 0.2* |
| | | | | Post filter | 0.41 | 94 | 98.3 | 1.5* | 0.2* |
| | | | | Pre filter | 0.44 | 102 | 98.3 | 1.5* | 0.2* |
| | | 1.5 | 100 | 0 | 1.30 | 97 | 98.4 | 1.4 | 0.2 |
| | | | | 24 | 1.30 | 97 | 98.4 | 1.4 | 0.2 |
| | | | | Post filter | 1.25 | 93 | 98.3 | 1.4 | 0.2 |
| | | | | Pre filter | 1.31 | 97 | 98.3 | 1.4 | 0.3 |
| | | 3.15 | 100 | 0 | 2.76 | 96 | 98.4 | 1.4 | 0.2 |
| | | | | 24 | 2.72 | 94 | 98.4 | 1.4 | 0.2 |
| | | | | Post filter | 2.70 | 94 | 98.4 | 1.4 | 0.2 |
| | | | | Pre filter | 2.75 | 95 | 98.4 | 1.4 | 0.2 |
| Bag | PP Bag | 0.48 | 60 | 0 | 0.46 | 101 | 98.3 | 1.4* | 0.2* |
| | Ecoflac | | | 24 | 0.45 | 100 | 98.3 | 1.5* | 0.3* |
| | plus ® | | | Post filter | 0.43 | 95 | 98.1 | 1.4* | 0.4* |
| | | | | Pre filter | 0.46 | 102 | 98.3 | 1.4* | 0.3* |
| | | 1.5 | 100 | 0 | 1.33 | 97 | 98.4 | 1.4 | 0.2 |
| | | | | 24 | 1.34 | 98 | 98.4 | 1.4 | 0.2 |
| | | | | Post filter | 1.29 | 94 | 98.4 | 1.4 | 0.2 |
| | | | | Pre filter | 1.32 | 97 | 98.4 | 1.4 | 0.2 |
| | | 3.15 | 100 | 0 | 2.77 | 97 | 98.4 | 1.4 | 0.2 |
| | | | | 24 | 2.74 | 96 | 98.3 | 1.4 | 0.2 |
| | | | | Post filter | 2.71 | 95 | 98.3 | 1.5 | 0.2 |
| | | | | Pre filter | 2.74 | 96 | 98.3 | 1.4 | 0.2 |

HMWS—High Molecular Weight Species,
LMWS—Low Molecular Weight Species,
*no LoQ assessed for the low dose LMW and HMW

### Conclusions and Recommendations

The compatibility of the diluted drug product (DP) was tested with contact surfaces of three infusion bags, two different infusion tubings and two different in-line filters to qualify representative clinical administration materials.

The physicochemical stability of the DP solution for infusion in the three infusion bag types (PE, PVC and PP) for up to 24 hours with exposure to both ambient and cold storage conditions (20 hours at 2-8° C. in the dark and 4 hours at ambient conditions with exposure to light) is supported by physicochemical analytical data. Bag infusion was performed over approximately 60 minutes with each infusion set (infusion bag, infusion line, in-line filter, stopcock and catheter).

For the infusion bag combination tested (PE bag, PE-line, PES positively charged filter; PVC bag, PVC line, PES neutral filter; PP bag, PVC line, PES neutral filter), no major changes were observed in the physicochemical analytical tests [appearance of the solution (turbidity, color), pH, purity by SE-HPLC] indicating good compatibility with the selected materials. All doses tested were practically free from visible particles. Subvisible particles were within the pharmacopoeial requirements after the simulated administration using an in-line filter. The in-line filter showed effective reduction in subvisible particles with all samples being within acceptance limits for infusion. Subvisible particle counts ≥25 μm were less than or equal to 600 counts per container and subvisible particle counts ≥10 μm were less than or equal to 6000 counts per container considering an infusion volume equal to 100 mL. The use of an in-line filter is highly recommended in the clinical setting.

The study covers a target concentration range from 0.5 mg/mL up to 3.0 mg/mL. A recovery (≥93%) of the concentration in Pre-filter and Post filter samples as compared to the theoretical initial concentration was obtained for all the dosage levels: low dose, intermediate dose and high dose (0.5 mg/mL, 1.5 mg/mL and 3.0 mg/mL) for all the material combinations tested (PE bag, PE-line, PES positively charged filter; PVC bag, PVC line, PES neutral filter; PP bag, PVC line, PES neutral filter).

DP is considered compatible with clinical components in the target concentration range of 0.5 mg/mL to 3.0 mg/mL in 5% dextrose in each of the following combinations:
Combination 1:
   Infusion Bag (PE contact material)
   Infusion line (PE contact material)
   In-line filter (PES, positively charged)
   a 3-way stopcock (PA contact) material
   Catheter (PUR contact material)
Combination 2:
   Infusion Bag (PVC contact material)
   Infusion line (PVC contact material)
   In-line filter (PES, neutral)
   3-way stopcock (PA contact material)
   Catheter (PUR contact material)
Combination 3:
   Infusion Bag (PP contact material)
   Infusion line (PVC contact material)
   In-line filter (PES neutral)
   3-way stopcock (PA contact material)
   Catheter (PUR contact material)

### Example 2: Treatment of Patients with Atrial Fibrillation with Antibody 1

### Brief Summary

This study was a multicenter, randomized subject and investigator-blinded, placebo-controlled, parallel=group, multiple ascending dose-ranging study to evaluate the safety, tolerability, pharmacokinetics (PK), and pharmacodynamics (PD) effects of Antibody 1 in patients with atrial fibrillation (AF) or atrial flutter at low risk of thromboembolic stroke or peripheral embolism. The trial evaluated the effects of up to three different doses of Antibody 1 on Factor XI (FXI) inhibition, indices of coagulation, and thrombogenesis biomarkers compared to placebo. The incidence of injection site reactions, bleeding events, immunogenicity, and systemic arterial and venous thromboembolic events were also assessed. Results from this study assist with dose-selection of Antibody 1 for a phase 3 trial in patients with AF.

### Detailed Description

This was a randomized, subject- and investigator-blinded, placebo controlled, dose-ranging study in patients with atrial fibrillation (AF) or atrial flutter at low risk for stroke. Patients were enrolled in up to 3 cohorts of approximately 16 patients each. After a Screening Period of up to 4 weeks, patients were randomized in a 3:1 ratio (Antibody 1:placebo) to receive 3 monthly subcutaneous (s.c.) injections and followed for pharmacokinetics, pharmacodynamic efficacy, and safety events over the 90-day Treatment Period. Patients were followed up to Day 170 during the Washout/Follow-up period.

### Inclusion Criteria

The following describes the inclusion criteria for patients enrolled in the clinical study described in this example. Patients:
have given written informed consent before any assessment is obtained;
are male and female patients ≥18 and <85 years old, with paroxysmal atrial fibrillation (PAF) or atrial flutter on 12 lead electrocardiography at screening; or have a history of PAF or atrial flutter, as documented by (telemetry, 12 lead electrocardiography or ambulatory [e.g., Holter] monitor) and not due to a reversible condition (e.g., alcohol binge drinking) even if they do not have PAF at Screening (there is no time limit for this);
have a CHA2DS2-VASc risk score (tool as a predictor for estimating the risk of stroke in patients with AF, Lip et al. 2010) of 0-1 for men and 1-2 for women and in whom, in the investigator's judgment, the use of an anticoagulant for stroke prevention is not indicated;
have a body weight between 50 and 130 kg, inclusive.

### Exclusion Criteria

The following describes the criteria that excluded patients from enrolling in the clinical study described in this example. Patients:
Using other investigational drugs at the time of enrollment, or within 5 half-lives of enrollment, or until the expected PD effect has returned to baseline, whichever is longer; or longer if required by local regulations;
have a history of stroke, transient ischemic attack, or systemic embolism;
have a history of major bleeding during treatment with an anticoagulant or antiplatelet therapy (patients who have had major bleeding on anticoagulants or antiplatelet therapy more than a year ago may be enrolled only if the bleeding was due to a reversible cause, e.g. a gastroduodenal ulcer that was successfully treated);
have a history of traumatic or non-traumatic intracranial, intraspinal, or intraocular bleeding;
have known bleeding diathesis or any known active bleeding events;
have had a myocardial infarction, unstable angina pectoris, or coronary artery bypass graft (CABG) surgery within 12 months prior to the Screening period;
have known clinically significant valvular heart disease including moderate or severe mitral stenosis (with valve area <1.5 cm²);
have a prosthetic heart valve;
have uncontrolled hypertension, defined as SBP/DBP≥160/100 mmHg at the screening visit
have New York Heart Association (NYHA) Class III-IV heart failure;
currently being treated with anticoagulant therapy or have been on anticoagulants in the previous 12 months; potential patients who have been on anticoagulation more than 12 months ago require discussion with the sponsor before enrolling;
receiving treatment with antiplatelet therapy such as a P2Y12 inhibitor or aspirin (a low dose of aspirin, defined as ≤100 mg/day, is allowed);
have severe renal impairment as defined as an estimated glomerular filtration rate ≤45 mL/min/1.73 m² by the Modification of Diet in Renal Disease (MDRD) equation at the Screening Visit
who test positive for human immunodeficiency virus (HIV), hepatitis B (hepatitis B surface antigen (HBsAg)), or hepatitis C (anti-hepatitis C antibody (Anti-HCV)) at Screening;
with significant illness, per Investigator judgment, which has not resolved within four week prior to dosing;
who are women of child-bearing potential, defined as all women physiologically capable of becoming pregnant, unless they are using highly effective methods of contraception during their time in the study. Highly effective contraception methods include:
   total abstinence (when this is in line with the preferred and usual lifestyle of the subject). Periodic abstincence (e.g., calendar, ovulation, symptothermal, post-ovulation methods) and withdrawal are not acceptable methods of contraception;
   female sterilization (surfical bilateral oophorectomy with or without hysterectomy), total hysterectomy, or tubal ligation at least six weeks before taking investigational drug. In the case of oophorectomy alone, only when the reproductive status of the woman has been confirmed by follow-up hormone level assessment;
   male sterilization of sexual partner (at least 6 months prior to screening). For female subjects on the study, the vasectomized male partner should be the sole partner for that subject;
   use of oral (estrogen or progesterone), injected or implanted hormonal methods of contraception or placement of an intrauterine device (IUD) or intrauterine system (IUS) or other forms of hormonal contraception that have comparable efficacy (failure rate <1%), for example, hormone vaginal ring or transdermal hormone contraception. In the case of oral contraception, women should have been stable on the same pill for a minimum of 3 months before taking the investigational drug.
Women are considered post-menopausal and not of child-bearing potential if they have had 12 months of natural (spontaneous) amenorrhea with an appropriate clinical profile (e.g. age appropriate, history of vasomotor symptoms) or have had surgical bilateral oophorectomy (with or without hysterectomy), total hysterectomy, or tubal ligation at least six weeks ago. In the case of oophorectomy alone, only when the reproductive status of the woman has been confirmed by follow-up hormone level assessment with follicle stimulating hormone (FSH) is she considered not of child-bearing potential. Male subjects must also agree to use highly effective methods of contraception during their time in the study and should not father a child or donate sperm in this period;
be pregnant or nursing (lactating) women, where pregnancy is defined as the state of a female after contraception and until the termination of gestation, confirmed by a positive human chorionic gonadotropin (hCG) laboratory test;
have a psychiatric disease or substance abuse history, which in the opinion of the Investigator could interfere with protocol compliance; or
have any surgical or medical condition, which in the opinion of the Investigator, may place the patient at higher risk from his/her participation in the study, or is likely to prevent the patient from compleying with the requirements of the study or completing the study.

### Dosage and Administration

A dose level was randomly assigned to each patient at trial entry. In certain embodiments, patients received 120 mg Antibody 1 via subcutaneous injection once every month, on Day 1 with two subsequent monthly injections. In certain embodiments, patients received 180 mg Antibody 1 via subcutaneous injection once every month, on Day 1 with two subsequent monthly injections. Antibody 1 via subcutaneous injection once every month, on Day 1 with two subsequent monthly injections. In certain embodiments, patients received placebo via subcutaneous injection once every month, on Day 1 with two subsequent monthly injections.

### Outcome Measures

The primary outcome measure was inhibition of FXI at trough (defined as the lowest concentration reached by Antibody 1) after the third dose (Day 91) for each dose cohort of Antibody 1, determined as the occurrence of achieving ≥50%, ≥80%, or ≥90% inhibition of FXI (<50%, <20%, or <10% free FXI) at trough on Day 91 for each dose cohort of Antibody 1. This was assessed at Day 91 of the study.

Secondary outcome measures included inhibition of FXI at trough (defined as the lowest concentration reached by Antibody 1 prior to administration of the next dose) after the first and second doses (Day 31 and Day 61, respectively) for each dose cohort of Antibody 1, determined as the occurrence of achieving ≥50%, ≥80%, or ≥90% inhibition of FXI (<50%, <20%, or <10% free FXI) at trough on Day 31 and Day 61 for each dose cohort of Antibody 1. This was assessed at Day 31 and Day 61 of the study.

Additional pre-specified outcome measures included analysis of occurrence of major cardiovascular, cerebrovascular, systemic arterial, and venous thromboembolic events (VTEs), to evaluate the effect of Antibody 1 compared to placebo on the incidence of major cardiovascular, cerebrovascular, systemic arterial, and venous thromboembolic events. Such events were recorded if they occurred on-treatment, from first dose of the study to the last dose of the study drug, plus 30 days if the subject permanently discontinued the study drug prior to the third dose on Day 91. Concentrations of D-dimer and other exploratory thrombogenesis biomarkers were also evaluated to determine change from baseline in D-dimer and other thrombogenesis biomarkers with Antibody 1 relative to placebo during the treatment period. These biomarkers were assessed at Screening and Days 1, 11, 31, 61, 71, 91, and 121.

### Safety Assessments

Physical examination, including assessment of general appearance, skin, lymph nodes, head, eyes, ears, nose and throat (HEENT), neck, thorax/lungs, cardiovascular, abdomen, musculoskeletal, and neurological systems were determined at Screening and Days 1, 11, 31, 61, 91, 121, and 170. Pre-dose values, post-dose values, and the change from baseline in vital sign measurements including sitting diastolic and systolic blood pressure, pulse, temperature, and body weight were determined at Screening and Days 1, 11, 31, 41, 61, 71, 91, 101, 121, and 170.

12-lead electrocardiogram (ECG) parameters were measured (QTc-Frederica, QT, RR, ventricular rate, PR, and QRS), listed, and summarized with descriptive statistics (n, mean, SD, median, minimum, and maximum) at each timepoint by treatment. Listings of subjects with abnormal ECG results, as judged by the Investigator, were displayed and calculated. The baseline was the value just prior to first administration of the study medication. These parameters were determined at Screening and Days 1, 91, and 170.

Safety laboratory analyses for hematology, clinical chemistry, and urine analysis were performed at Screening and Days 1, 11, 31, 41, 61, 91, 121, and 170.

Hypersensitivity reactions and injection site reactions were monitored by assessing symptoms or signs consistent with an injection site reaction or hypersensitivity reaction at Days 1, 11, 31, 41, 61, and 71.

Adverse Events (AEs) including Serious Adverse Events (SAEs), including incidence, severity, relationship, duration, and determination if the event is an SAE, were recorded. Any AE or SAE occurring from Screening up to the end of the study (Day 170) was be recorded.

Occurrence of confirmed major bleeding events, clinically relevant non-major (CRNM) bleeding events, and total bleeding with Antibody 1 relative to placebo during the treatment period was recorded. Such bleeding events were recorded if they occur on-treatment, from first dose of the study to the last dose of the study drug, plus 30 days if the subject permanently discontinued the study drug prior to the third dose on Day 91.

Screening and confirmation of anti-drug (anti-Antibody 1) antibodies (ADA) were assessed to evaluate the immunogenicity of Antibody 1 compared to placebo. Screening was performed at Days 1, 31, 61, 71, 91, 121, and 170.

### Biomarker Assessment

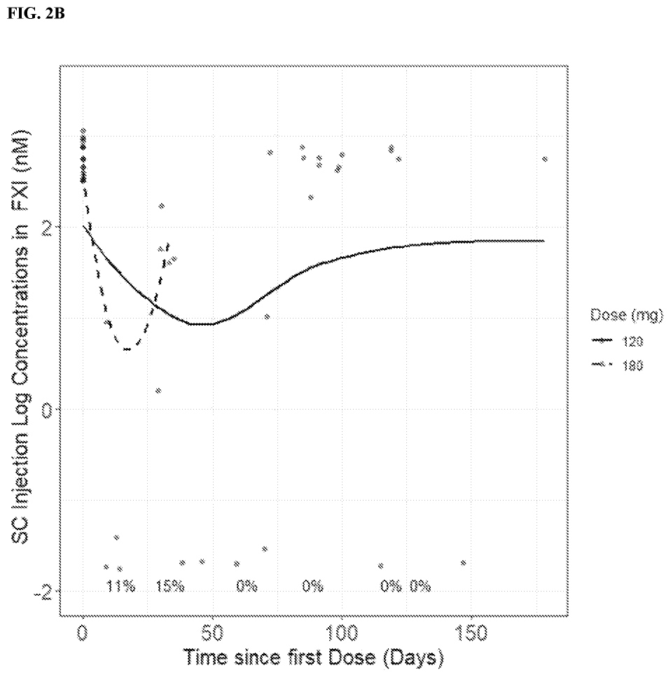

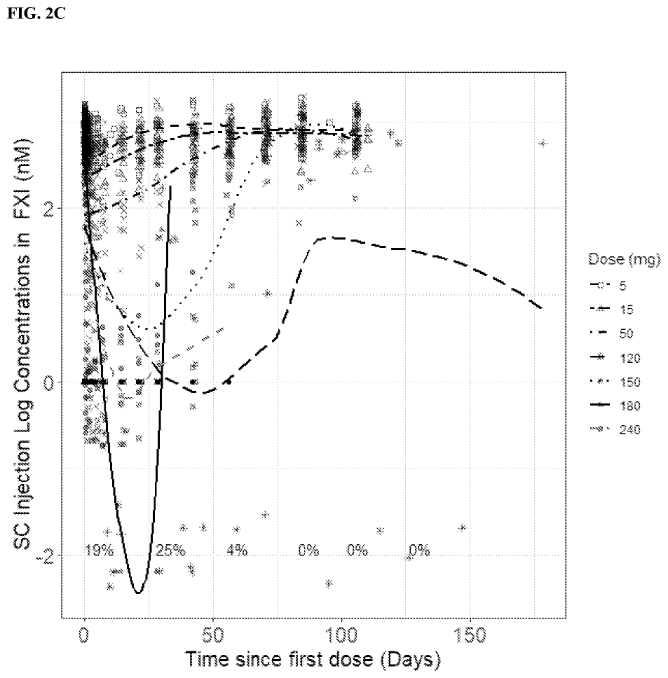

Results of this study demonstrated high efficacy of Antibody 1. As shown in FIG. 2A, peak levels of Antibody 1 in plasma demonstrated a gradual decline at the 120 mg dose, highlighting the long-half life of the antibody. Similarly, free Factor XI observed in serum showed a prolonged decrease for the 120 mg dose (FIG. 2B). When Antibody 1 was administered in multiple doses (i.e., 120 mg and 180 mg), a significantly longer reduction of plasma free FXI was observed as compared to single dose (FIG. 2C). For each Figure, dots represent the observed data, lines represent the smoothing “loess” curve (indicating the relationship between 2 variables), and the numbers at the bottom represent percentage of BLQ records at each time group.

### Example 3: Treatment of Patients Undergoing Elective Unilateral Total Knee Arthroplasty (TKA) with Antibody 1

### Purpose and Rationale

The purpose of this study was to provide an early indication of antithrombotic efficacy of Antibody 1 versus (vs.) standard of care (enoxaparin) for prevention of postoperative venous thromboembolism (VTE) in patients undergoing elective unilateral total knee arthroplasty (TKA).

In two Phase 1 single ascending dose studies, Antibody 1 appeared to be safe and well tolerated, producing robust and sustained Factor XI (FXI) inhibition and prolongation of the activated partial thromboplastin time (aPTT) for 4 weeks or longer at relevant doses.

3 doses of Antibody 1 intravenously administered (i.v.) were studied (30 mg, 75 mg, and 150 mg). These doses were predicted to have ≥43%, ≥97% and >99% reductions of free FXI from baseline in 90% of subjects at Day 10, and >7%, ≥18%, and >93% reductions in 90% of subjects at Day 30.

All doses were expected to provide efficacy in terms of prevention of VTEs.

### Objectives

The primary objective of this study was to assess if at least one dose of Antibody 1 is non-inferior to enoxaparin 40 mg through Day 10 after randomization in terms of incidence of adjudicated total VTE in patients undergoing unilateral TKA. Non-inferiority (NI) was met, and superiority was subsequently tested.

The secondary objectives of this clinical study were:
to evaluate the effect of Antibody 1 relative to enoxaparin in terms of incidence of adjudicated major and clinically relevant non-major (CRNM) bleeding through Day 10 and through Day 30 after randomization; and
to assess if at least one dose of Antibody 1 is non-inferior to enoxaparin 40 mg through Day 30 after randomization and end of study (EoS) visit in terms of incidence of adjudicated total VTE in patients undergoing unilateral TKA. If non-inferiority is met, superiority will be tested. Non-inferiority will be tested by assessment of occurrence of confirmed composite endpoint of asymptomatic DVT, confirmed symptomatic VIE, fatal and non-fatal PE, or unexplained death for which PE could not be ruled-out during treatment through Day 30 and Day 110.

The exploratory objectives of this clinical study were:
to evaluate the effect of Antibody 1 relative to enoxaparin in terms of incidence of major and CRNM bleeding events through Day 10 and Day 30, to be assessed by occurrence of confirmed composite endpoint of major and CRNM bleeding through Day 10 and Day 30.
to evaluate the effect of Antibody 1 relative to enoxaparin in terms of incidence of major bleeding events, CRNM bleeding events, and total bleeding events through Day 10 and Day 30, to be assessed by occurrence of confirmed composite endpoint of major and CRNM bleeding through Day 10 and Day 30.
to evaluate the effect of Antibody 1 relative to enoxaparin in terms of incidence of major bleeding events, CRNM bleeding events, and total bleeding events through Day 10 and Day 30, to be assessed by occurrence of confirmed composite endpoint of major and CRNM bleeding through Day 110.
to evaluate the effect of Antibody 1 relative to enoxaparin in terms of incidence of major bleeding events, CRNM bleeding events, and total bleeding events through Day 10 and Day 30, to be assessed by the proportion of patients requiring transfusion, through Day 30 and EoS visit, and the number of blood units transfused through Day 30 and the Day 110/EoS visit.

### Study Design

This was a randomized, open-label, blinded endpoint adjudication study with a screening period of up to 30 days, a treatment period of 10±2 days and a follow-up period to Day 110. Each randomized patient underwent TKA surgery on Day 1 of the study. During the treatment period, patients received Antibody 1 on Day 1, after surgery or daily subcutaneous (s.c.) injections of enoxaparin up to the Day 10 (±2 days) visit. Unilateral venography of the operated leg was completed on Study Day 10 (±2 days). Completion of venography concluded the Treatment Period of the study. After the Treatment Period, patients entered the Follow-up Period of the study.

### Antibody 1

Antibody 1 was provided as a liquid in vial concentrate (150 mg/mL). Antibody 1 was stored as per the information provided on the label, at 2-8° C., and should not be frozen. Antibody 1 was prepared by the Pharmacist or qualified pharmacy delegate for i.v. infusion.

### Enoxaparin

Enoxaparin (enox) 40 mg s.c. was used as comparator, in accordance to the local country requirements and regulations. Storage conditions described in the prescribing information were followed.

### Population

600 male and female patients aged ≥18 years who need elective unilateral knee arthroplasty were included in this study. Approximately 20% of randomized patients were anticipated to have a non-evaluable venography; consequently, approximately 600 patients (150 patients per group) were randomized to ensure that 480 patients were evaluable for the primary study endpoint. If the rate of non-evaluable patients appears to be different from the anticipated 20%, the number of randomized patients will be adjusted to ensure that 480 patients will be evaluable for the study primary endpoint.

### Inclusion Criteria

The following describes the inclusion criteria for patients enrolled in the clinical study described in this example. Patients:
are male and female patients (aged ≥18 years and <80 years old)
are scheduled to undergo elective unilateral TKA;
are able to comprehend and willing to give written informed consent;
are willing to comply with study requirements including venography at Day 10±2 days;
have body weight between 50 and 130 kg, inclusive; and
have aPTT and PT/INR within the upper limits of normal (ULN) at Screening.

### Exclusion Criteria

The following describes the criteria that excluded patients from enrolling in the clinical study described in this example. Patients:
who have used other investigational drugs within 5 half-lives of enrollment, or until the expected pharmacodynamic effect has returned to baseline, whichever is longer;
with a history of hypersensitivity to any of the study drugs (including enoxaparin) or its excipients, to drugs of similar chemical classes, or to drugs issued from the same biologic origin or any contraindication listed in the label for enoxaparin;
with an indication for full-dose anticoagulation or dual antiplatelet therapy or anticipated concomitant use of anticoagulant/antiplatelet agents that have the potential to affect study outcome or any other drug influencing coagulation (except low dose aspirin and short-acting NSAIDs) at least 7 days before surgery to the EoS visit;
with known or suspected active bleeding at study entry;
with urine protein or blood persistently positive by dipstick;
at increased risk of bleeding because of a history of increased bleeding tendency (e.g., history of bleeding diathesis, known active gastrointestinal lesions such as angiodysplasia or an endoscopically verified gastrointestinal ulcer or a history of gastrointestinal bleeding within the past year) or any other condition that in the opinion of the Investigator increases risk of bleeding, or patients with a history of intracranial or intraocular bleeding;
who have undergone major surgery including brain, spinal, or ophthalmologic surgery within the past 6 months;
with a history of a traumatic spinal or epidural anesthesia or excessive intra- or direct postoperative bleeding;
who have suffered major trauma within the past 6 months;
with a history of VTE;
who have experience malignancy within the past year, except for basal or squamous cell carcinoma of the skin, or carcinoma in situ of the cervix that has been successfully treated;
who have had a myocardial infarction, stroke, or transient ischemic attack in the past 6 months;
with uncontrolled hypertension as judged by the Investigator;
with estimated glomerular filtration rate (eGFR)<60 mL/min/1.73 m²;
with clinically significant anemia during screening as judged by the Investigator;
with platelet count <150,000 m³ at screening, or a history of heparin-induced thrombocytopenia;
who are unable to undergo venography due to a known allergy to the contrast agent, anticipated poor venous access, impaired renal function, or any other reason identified and specified by the Investigator;
with anticipated use of intermittent pneumatic compression devices post TKA procedure;
with liver dysfunction (ALT/AST >3× ULN or total bilirubin >2× ULN), a diagnosis of liver cirrhosis, history of hepatic encephalopathy, esophageal varices, or portocaval shunt;
who test positive for human immunodeficiency virus (HIV), positive hepatitis B (hepatitis B surface antigen [HBsAg]) or hepatitis C (anti-hepatitis C antibody [Anti-HCV]) at Screening;
with clinically significant abnormal ECG at Screening as judged by the Investigator;
with recent or current history of alcoholism or drug addiction;
who are pregnant or nursing (lactating) women;
who are women of child-bearing potential, defined as all women physiologically capable of becoming pregnant, unless they are using highly effective methods of contraception;
who have experienced significant illness which has not resolved within two (2) weeks prior to the start of the study drug;
who have experienced any illnesses or other medical conditions which may preclude patients from complying with study requirements; or
with anticipated elective surgery (e.g. contralateral TKA) during the study period.

### Dosage and Administration

A dose level was randomly assigned to each patient at trial entry. In certain embodiments, patients received 30 mg Antibody 1 intravenously (i.v.) once every month. In certain embodiments, patients received 75 mg Antibody 1 i.v. once every month. In certain embodiments, patients received 150 mg Antibody 1 i.v. once every month. In certain embodiments, patients received 40 mg enoxaparin subcutaneously (s.c.) once every day. Antibody 1 was administered approximately 4 to 8 hours after TKA surgery. Enoxaparin was administered starting approximately 12 hours after TKA surgery, followed by daily s.c. injections of 40 mg enoxaparin up to the Day 10 visit venography. A single initial 40 mg s.c. enoxaparin dose prior to TKA surgery was given at the discretion of the investigator per local guidelines.

### Efficacy and Safety Assessments

Efficacy assessments: Efficacy was assessed by occurrence of composite endpoint of adjudicated asymptomatic deep vein thrombosis (DVT) as detected by unilateral ascending venography of the operated leg, confirmed symptomatic DVT, fatal and non-fatal pulmonary embolism (PE), or unexplained death for which PE could not be ruled out throughout the treatment period through Day 10 (day of venography). Venography readers and central adjudicators of the composite endpoint were blinded to treatment allocation.

Patients had study visits on Day 3, Day 6, and Day 10 during the Treatment period. The duration of the hospital stay after surgery was at the investigator's discretion and according to local medical practice. If patients were discharged before Day 10, they should return to the investigational site for a clinical evaluation and for the venography on Day 10±2. If a patient was discharged on or prior to Day 3, the assessments scheduled at the Day 3 and Day 6 visits were be collected, but not duplicated. If a patient was discharged on Day 4 (prior to the Day 6±1 study visit, but after Day 3 assessments have already been completed), sites collected the Day 6 assessments prior to discharge.

Patients were evaluated for signs and symptoms of DVT (swelling, localized pain, redness, heat, localized warmth) and non-fatal PE (i.e. unexplained shortness of breath, chest pain that gets worse with a deep breath, coughing or chest movement, or coughing up blood) during the hospital stay and at all visits post discharge.

Every suspected episode of DVT was documented by prompt objective confirmation by compression ultrasound (CUS) or unilateral or bilateral venography. When a symptomatic proximal DVT was objectively confirmed by CUS prior to the scheduled venography (Day 10±2), the venography can be omitted. When a suspected DVT was not confirmed by CUS, the venography should be scheduled and performed. If a DVT was suspected on the day of the venogram, the venogram can be performed as scheduled. Only when a suspected symptomatic DVT was objectively confirmed by CUS, can the venography be omitted.

Every suspected episode of PE was confirmed by ventilation/perfusion lung scintigraphy, spiral computed tomography, or pulmonary angiography.

If the presence of DVT or PE was confirmed using the above techniques during the Treatment Period, no venogram was done on the Day 10 visit. If a DVT or PE was not confirmed using the above techniques, the patient must undergo the venography.

Mandatory venography was performed on the Day 10 visit if not otherwise specified as described above. The venography images or images of the diagnostic test for suspected symptomatic DVT and PE were collected and transferred as soon as possible for review by the Central Independent Adjudication Committee (CIAC) whose members are kept blinded to treatment assignment.

During the Follow-up Period, patients had additional visits on Day 30, Day 50, and Day 110 where additional safety laboratory parameters, pharmacokinetics (PK), and pharmacodynamics (PD) were assessed. The End-of-Study (EoS) visit took place on Day 110.

During the study, venography, all suspected VTE, all suspected bleeding events, and unexplained deaths were adjudicated on an ongoing basis by the CIAC. Aggregated data for total VTE (asymptomatic DVT, symptomatic VTE, and PE related deaths), total bleeding events, and other safety events unblinded to treatment assignment were reviewed at regular intervals by the Steering and Safety Committee (SSC) which has overall clinical and scientific responsibility for the trial. This data was kept confidential from the clinical trial team directly involved in study conduct. The primary purpose of this review was to drop the low dose, or the low and middle doses if the rate of VTE was unacceptable.

Efficacy was assessed by occurrence of confirmed composite endpoint of asymptomatic DVT as detected by the protocol-required unilateral venography of the operated leg at Day 10±2, confirmed symptomatic DVT, fatal and non-fatal PE, or unexplained death for which PE could not be ruled-out through Day 30 after the randomization and the EoS visit.

Key safety assessments: Occurence of adjudicated composite outcome of major bleeding and CRNM bleeding events though the treatment period and through Day 30 was monitored. Adverse event (AE) occurences, physical examinations, hypersensitivity reactions and injection site reactions (ISRs), and laboratory markers in blood and urine were monitored.

Other assessments: Total Antibody 1 was measured at (pre-surgery) Day 1, Day 3, Day 6, Day 10, Day 30, Day 50, and Day 110 (EoS visit) for pharmacokinetics (PK) determination. Post-treatment concentrations of Antibody 1 was used to derive the following PK parameters: Cₘₐₓ, Tₘₐₓ, AUCₗₐₛₜ, Cₗₐₛₜ and Tₗₐₛₜ. If feasible, AUC_{inf}, T_{1/2}, V_{d/F}, and C_{L/F}. The presence of antidrug antibodies was measured at (pre-surgery) Day 1, Day 30, Day 50, and Day 110 (EoS visit).

Coagulation biomarkers, including aPTT and PT/INR was measured at Screening, (pre-surgery) Day 1, Day 3, Day 6, Day 10, Day 30, Day 50 and Day 110 (EoS visit); calibrated aPTT and FXI:C was measured at (pre-surgery) Day 1, Day 3, Day 30 and Day 50; and free and total FXI was measured at (pre-surgery) Day 1, Day 3, Day 6, Day 10, Day 30, Day 50 and Day 110 (EoS visit).

Data analysis: the primary Stratified Cochran-Mantel-Haenszel (CMH) test efficacy analysis was used to test the null hypothesis that differences in incidence rates of adjudicated total VTE events between Antibody 1 low, middle, and high dose and enoxaparin are equal or greater than the pre-specified NI margin (14%) versus the alternative hypothesis that the at least one difference is less than NI margin. The primary analysis was based on a 0.025 overall significance level (one-sided test) for the final analyses together for each comparison. For the non-inferiority test, the confidence interval (CI) approach was employed, and the 95% CI will be presented for comparisons of low, middle, and high dose Antibody 1 with enoxaparin. Non-inferiority was achieved since the upper limit of the confidence interval of incidence difference is less than or equal to the NI margin. The superiority test was performed non-inferiority has been shown. In addition, p-values (based on one-sided test) for both the non-inferiority and superiority tests were presented.

Analysis for efficacy was done in the mITT-set. This population consisted of all patients who have received at least one dose of study medication and who can be evaluated for the primary efficacy outcome (i.e., having an evaluable venography or a confirmed symptomatic DVT, fatal or non-fatal PE or unexplained death). Multiple comparison procedure was not be considered in statistical analyses; however, statistical testing was hierarchical.

### Risks and Adverse Event Monitoring

Risks Based on Prior Clinical Experience with Antibody 1

Antibody 1 has been generally well-tolerated in the two clinical studies completed to date. Adverse events (AEs) have been infrequent, mild in severity, and well-balanced between the treatment and placebo arms. No dose-dependent safety findings have been observed. While fecal occult blood tests have been positive in a few subjects, none of these positive test results remained positive on repeat testing or were associated with any clinical or laboratory signals indicating active bleeding. Thus, no specific risks have been identified based on prior clinical experience for Antibody 1.

### Risks Based on Antibody 1 Preclinical Toxicity Studies

While specific safety pharmacology studies have not been conducted, neither qualitative nor quantitative electrocardiographic changes nor effects on respiratory and central nervous system functions attributable to Antibody 1 administration were observed in the 13-week toxicity study. No mortality occurred and there were no test article-related effects on clinical signs, body weight, food consumption, ophthalmologic and electrocardiographic parameters, hematology, clinical chemistry, urinalysis, macroscopy, or histology and no occult blood was detected in the feces. No adverse findings were identified in this study. Based on the above data, no specific anticipated risk was identified.

### Risk Based on the Biology of Antibody 1

In the 13-week Good Laboratory Practice(GLP)-compliant toxicity study, no bleeding manifestations, venous puncture or injection site bleeding was observed following treatment with Antibody 1 up to a weekly dose of 100 mg/kg s.c. or 50 mg/kg i.v.

Treatment with Antibody 1 is expected to result in a bleeding phenotype comparable to the bleeding phenotype in patients with severe FXI deficiency. Unlike hemophilia A and B, FXI deficiency is rarely associated with spontaneous bleeding manifestations. Bleeding manifestations in subjects with severe FXI deficiency are infrequent, often mild, and injury-induced and preferentially affect tissues with increased fibrinolytic activity such as the oral mucosa, nasal mucosa and urinary tract. Spontaneous muscle or joint bleeding or intracranial bleeding is rare with FXI deficiency (Bolton-Maggs 2000, Duga and Salomon 2013).

An FXI-antisense oligonucleotide (ASO) was also evaluated in a first-in-human (FIH) study in healthy subjects (Liu et al 2011) and in patients undergoing elective unilateral total knee arthroplasty (Buller et al 2015). Robust and sustained reductions of free FXI >80% were achieved for a duration exceeding 6 to 8 weeks at the highest dose (several subjects reaching undetectable levels). No bleeding occurred in the 88 subjects who received FXI-ASO or placebo in the FIH study. Furthermore, administration of FXI-ASO to patients undergoing unilateral TKA at a dose that achieved 80% reduction in FXI: C appeared to be associated with a trend toward a lower incidence of major or CRNM bleeding events compared to low dose enoxaparin. One major bleeding event (1% of patients) occurred in the high dose FXI-ASO group (Buller et al 2015). To mitigate the bleeding risk following treatment with Antibody 1, subjects at increased risk for bleeding events are excluded from this trial.

Potential Risk Associated with Hypersensitivity Reactions

Infusion of therapeutic proteins can result in immediate or delayed hypersensitivity reactions. Immediate reactions appear during the first hours after drug administration. Clinical presentation may include a wide of range of symptoms e.g., fever, chills, nausea, cutaneous symptoms, bronchospasm, dyspnea, dizziness, headaches, myalgia, tachycardia and/or hypotension. Anaphylaxis, urticaria, and angioedema were also reported. Delayed hypersensitivity reactions can appear between 1-2 hours and up to 14 days after administration, often with serum sickness—like symptoms (Corominas et al. 2014). The incidence of hypersensitivity reactions with monoclonal antibodies (mAbs) depends on the degree of humanization, the cell line from which they were obtained and excipients. No hypersensitivity reactions were observed in the 13-week toxicity study and in the FIH study.

Management of hypersensitivity reactions depends on the clinical presentation and severity. Treatment interruption (if applicable); fluids, vasopressors, corticosteroids, antihistamines, bronchodilators, epinephrine and oxygen may be used, as indicated.

Potential Risk Associated with Complement Dependent Cytotoxicity (CDC) or Antibody-Dependent Cell-Mediated Cytotoxicity (ADCC)

IgG1 mAbs bind Fc gamma receptors (FcγRs) and human complement 1 q subcomponent (Clq) and thus have potential for Fc-mediated effector function. In ADCC, mAbs interact directly with FcγR-expressing cells. In CDC, mAbs interact with Clq, leading to activation of the complement system and release anaphylatoxins and opsonins.

Although FXI is a soluble target, it can be cell-surface associated, most notably on the surface of activated platelets where it binds to the platelet glycoprotein Ib α receptor. Two Fc receptor function silencing mutations were introduced in the Fc domain Antibody 1 to mitigate any potential risk of altering platelet function due to possible Antibody 1 clustering via platelet-bound FXI. These two mutations have been shown to reduce binding of IgG1 s to FcγRs and C1q and to reduce ADCC and CDC in vitro (Idusogie et al 2000; Shields et al 2001). Therefore, Antibody 1 is expected to have a low risk of causing ADCC and CDC.

Potential Risks Associated with Study Procedures

Blood samples were collected frequently during the study either via venipuncture or i.v. cannula. Risks associated with blood collection include pain, swelling and/or bruising at the insertion site of the needle. Although rare, localized clot formation, infections and nerve damage may occur. Lightheadedness and/or fainting may also occur during or shortly after the blood draw. No more than 200 mL of blood was collected over a period of approximately 3.5 months from each patient randomized as part of the study. This estimate of blood collection did not include the collection of additional samples at unscheduled visits for monitoring of any safety findings. This volume of collection was not considered to be a risk for this patient population.

Performing a venography takes between 30 and 90 minutes. Local complication such as injection site bleeding, bruising or infection may occur following venography. Side effects of radiographic contrast media range from mild inconvenience, such as itching, to life-threatening emergency. Contrast-induced nephropathy is a well-known adverse reaction associated with the use of i.v. or intra-arterial contrast material. Other forms of adverse reactions include delayed hypersensitivity reactions, anaphylactic reactions, and cutaneous reactions.

To mitigate the risks of the use of contrast media, patients with known history of intolerance to contrast media as well as patients with moderate to severe renal impairment were excluded from study participation. Adequate hydration of the patient was applied during and after performing venography.

If patients experienced post-surgery renal impairment (defined as eGFR<45 mL/min or a 25% drop from baseline at the Day 6 visit), venography on Day 10 was not performed to minimize the risk of contrast induced nephropathy.

Potential Benefits Associated with Antibody 1 Use

Patients with severe FXI deficiency are at lower risk for venous thrombosis and stroke than the general population (Salomon et al 2008, Salomon et al 2011, Preis et al 2017). Furthermore, FXI suppression by 80% in average, using FXI ASO, resulted in superior efficacy to enoxaparin in terms of total VTE in patients undergoing unilateral TKA (Buller et al 2015). FXI ASO suppression was not associated with excessive bleeding during TKA surgery even with undetectable FXI levels in some patients (Buller et al 2015).

Antibody 1 is an investigational drug and the clinical benefits have not been established; however, early clinical data suggest that inhibiting FXI could be beneficial as an antithrombotic agent. Antibody 1 at the 150 mg dose and higher resulted in an average aPTT prolongation >2-fold and an average FXI inhibition >95% for >4 weeks in healthy subjects in the FIH study. The preclinical data and data from the FIH study showed an acceptable safety profile. The extended duration of action of Antibody 1 may provide additional advantage in term of VTE protection compared to enoxaparin (Falck-Ytter et al 2012).

### Treatment Assignment and Randomization

After successful completion of screening as per protocol, the patients were randomized to one of the following 4 treatment arms in a ratio of 1:1:1:1.
Antibody 1 30 mg i.v.×1
Antibody 1 75 mg i.v.×1
Antibody 1 150 mg i.v.×1
Enoxaparin 40 mg s.c. daily until the Day 10 venography

Randomization numbers were assigned in ascending, sequential order to eligible patient. The investigator entered the randomization number on the CRF. Randomization numbers were generated by or under the responsibility of Covance using a validated system that automates the random assignment of treatment arms to randomization numbers in the specified ratio.

The randomization scheme for patients was reviewed and approved by a member of the Covance Randomization Group. The randomization number was used to link the patient to a treatment arm and to specify a unique medication number for the Antibody 1 to be dispensed for the patient. The randomization numbers were generated to ensure that treatment assignment is unbiased.

This is an open-label blinded endpoint evaluation design study. Investigators and patients will have full knowledge of the treatment assignment with regards to Antibody 1 compared to enoxaparin. The study's primary safety and efficacy endpoints were adjudicated by the CIAC whose members remained blinded to treatment assignment. Randomization information was not be released to the clinical trial team prior to database lock.

### Treating the Patient

Antibody 1 was administered to the patient by qualified medical personnel via i.v. infusion (Antibody 1).

Each study site was supplied with Antibody 1 as open-label, single use vials. A pharmacist or pharmacy designee prepared study drug for administration in accordance with the treatment assignment. All Antibody 1 doses were administered in dextrose 5% in water (D5W) over approximately 1 hour.

Enoxaparin for s.c. administration was sourced locally by the individual study site or supplied open-label through the Sponsor, or delegate working on the sponsor's behalf, in accordance with local guidelines.

Antibody 1 was prepared by the Pharmacist or designated site staff and administered approximately 4-8 hr post TKA surgery i.v. as a single infusion over approximately 1 hour.

Enoxaparin 40 mg was administered s.c. starting approximately 12 hr after TKA surgery followed by daily s.c. injections of 40 mg enoxaparin up to the visit Day 10 venography. A single initial 40 mg s.c. enoxaparin dose prior to TKA surgery was given at the discretion of the investigator per local guidelines. Prolongation of enoxaparin treatment beyond the Day 10 venography was at the investigator's discretion and local medical practice. Table 12 describes the administration of Antibody 1 and enoxaparin.

TABLE 12
Administration of Antibody 1 and enoxaparin

| | Antibody 1 30 mg, 75 mg |
|---|---|
| Enoxaparin 40 mg group | or 150 mg dose group |
| First administration starting approximately | Single i.v. administration |
| 12 hours after TKA surgical wound closure, | approximately 4-8 hours |
| then q.d. until the Day 10 venography. (If | after surgical wound |
| consistent with local medical practice, the | closure |
| first dose of enoxaparin can be administered | |
| the day before surgery.) | |

If adjustments to the infusion rate (Antibody 1) or interruptions in dosing (either Antibody 1 or enoxaparin) were required to manage patient adverse reactions or care, details of the changes to the infusion rate/injection were recorded in the CRF. The sponsor should be notified if any of the following occur:
Antibody 1 infusion time <30 mins. or ≥2 hours or infusion interruption for ≥60 minutes
Total drug administered <90% or ≥110% of the allocated dose for either enoxaparin or Antibody 1

For patients in the enoxaparin treatment arm, in the event the patient was unable to tolerate enoxaparin treatment, a temporary interruption or permanent discontinuation of enoxaparin was considered at the investigator's discretion and should be documented in the appropriate CRF.

### Study Completion and Discontinuation

A patient was considered to have completed the study when the patient had completed the last visit planned in the protocol.

Discontinuation of study treatment for a patient occurred when study drug was stopped earlier than the protocol planned duration. Study drug discontinuation was initiated by either the patient or the Investigator.

The Investigator discontinued study treatment for a given patient if, on balance, he/she believed that continuation would negatively impact the risk/benefit of trial participation.

Study treatment was discontinued under the following circumstances: patient request, pregnancy, use of prohibited treatment, any situation in which study participation might result in a safety risk to the patient, or any laboratory abnormalities that in the judgment of the investigator, taking into consideration the patient's overall status, prevented the patient from continuing participation in the study

Patients treated with Antibody 1 received only one dose of the drug during the trial; therefore, discontinuation of treatment was not possible. Given the half-life of Antibody 1, the Investigator closely monitored aPTT levels during the Treatment Period and Follow-up Period.

If a bleeding event occurred in patients treated with enoxaparin, the investigator evaluated the clinical situation, whether drug administration can be delayed, temporarily stopped or permanently discontinued. Management of bleeding events in patients treated with enoxaparin was at the Investigator's discretion and in line with local clinical practice.

Platelets were closely monitored using a local laboratory facility in line with the usual medical practice. Enoxaparin treatment was discontinued if the platelet count fell below 100,000/mm³ or decreased by more than 50%, and assessment for heparin induced thrombocytopenia took place. Continuation of preventive treatment of VTE was at the investigator's discretion and should be in line with local clinical practice.

If a confirmed symptomatic VTE event developed in patients treated with enoxaparin, a therapeutic dose of enoxaparin followed by a therapeutic dose of a direct oral anticoagulant (DOAC) or warfarin for an appropriate duration of time was considered by the Investigator and in line with local clinical practice.

### Procedures and Assessments

The assessment schedule, shown in Table 13, details timing and exams to be assessed during the trial.

### Screening Period (Day −30 to Day −1)

A written informed consent for the study was obtained prior to performing any study-related procedures.

After identifying a potential patient, an informed consent form (ICF) was by the patient before performing any study-related screening assessments. The AE and SAE reporting period began at the time the ICF is signed.

Up to a 4-week period was provided for completing screening assessments and determining patient eligibility for the study. During the screening period, patients underwent a medical history and physical examination including vital signs and 12-lead ECG. Blood and urine samples were taken for clinical laboratory testing and other screening assessments.

Re-screening of patients was allowed if a patient has borderline abnormal laboratory values and/or if the surgery was rescheduled. Re-screening a patient was only allowed once during the study.

TABLE 13
Assessment Schedule

| | | Treatment Period | | | | Follow-up Period | | |
|---|---|---|---|---|---|---|---|---|
| Period | Screening | 1# | | 6§ | 10§ | 30 | 50 | 110 (EoS) |
| Day | Day −30 to −1 | (TKA) | 3§ | (±1) | (±2) | (±3) | (±5)¹ | (±5) |
| Obtain informed consent* | X | | | | | | | |
| Demography/medical history | X | | | | | | | |
| Inclusion/exclusion criteria | X | X | | | | | | |
| Physical exam | Complete | Abbrev. | | | Abbrev. | | | Abbrev. |
| | (S) | (S) | | | (S) | | | (S) |
| Height | X | | | | | | | |
| Weight | X | | | | | | | X |
| Vital signs | X | X | X | X | X | X | X | X |
| 12-lead ECG | X | | | | | | | X |
| HIV, Hepatitis B, Hepatitis C | S | | | | | | | |
| Drug, EtOH | S | | | | | | | |
| aPTT/PT/INR | X | X | X | X | X | X | X | X |
| Free and total FXI | | X | X | X | X | X | X | X |
| Calibrated aPTT and FXI:C | | X | X | | | X | X | |
| Exploratory blood biomarkers | | X | | | X | X | X | |
| PK assessment Antibody 1\hat{ } | | X | X | X | X | X | X | X |
| Antidrug antibodies | | X | | | | X | X | X |
| Hematology and Chemistry | X | X | X | X | X | X | X | X |
| Urine dipstick | X | X | | | X | X | X | X |
| Pregnancy test⋄ | Serum | Urine | | | Urine | Urine | | Urine |
| FSH+ | X | | | | | | | |
| Fecal occult blood testing | X | | | | | | | |
| Check for signs and symptoms of | | X | | | | | | |
| VTE | | | | | | | | |
| Check for any bleeding events | | X | | | | | | |
| Injection/infusion site inspection | | X† | X | X | X | | | |
| Surgical site bleeding assessment | | X† | X | X | X | | | |
| Administer Antibody 1 i.v. once 4-8 | | X† | | | | | | |
| hours after surgery | | | | | | | | |
| Administer enoxaparin s.c. 12 hours | | X† | | | | | | |
| after surgery then daily up to Day 10 | | | | | | | | |
| or longer per PI discretion | | | | | | | | |
| Venography | | | | | X | | | |
| AE assessment | X | | | | | | | |
| Concomitant medications | X | | | | | | | |
| Study completion CRF | | | | | | | | X |

Abbrev. = abbreviated physical exam
X = assessment to be recorded on clinical data base;
S = assessment to be recorded as source data
*ICF obtained prior to any study specific screening procedures.
# Day 1 visit (TKA surgical day) will be considered the reference visit for all study visits during the treatment and follow-up period. Day 1 assessments are performed before TKA surgery unless otherwise indicated with a †. Patients must be seen for all visits on the designated day or as close to it as possible.
§If a patient is discharged on Day 4 (prior to the Day 6 ± 1 study visit, but after Day 3 assessments have already been completed), sites should collect the Day 6 assessments prior to discharge. All patients who are discharged before Day 10 (±2 days) should return to the investigational site for the Day 10 visit for clinical investigation and the venography assessment.
\hat{ }Blood for PK and AD As will only be analyzed for those randomized to Antibody 1
⋄All female study participants, regardless of child-bearing status will have pregnancy testing. Serum pregnancy test will be done at screening. Urine pregnancy test will be done during rest of the visits. On Day 1, before drug administration, a positive urine pregnancy test will require confirmation by serum pregnancy test to determine continuation in the study. A positive serum pregnancy test on Day 1 will lead to immediate study discontinuation and complete pregnancy reporting as per Section 8.7. The same procedure will be followed for all other specified visits where urine pregnancy is tested. If on Day 10 before venography, the patient is confirmed to be pregnant, the venography should not be performed.
+Female patients only: required to confirm post-menopausal status and hormonal status in women who underwent oophorectomy without hysterectomy.
¹Due to the COVID-19 pandemic, the day 50 visit was turned into a telephone visit.

Re-screening assessments were collected up until immediately before the TKA surgery on Day 1. Re-screening lab samples were sent to the local lab for expedited turnaround time in this situation. All results were available prior to the TKA surgery for the patient to be enrolled into the study.

### Treatment Period (Day 1 to Day 10±2)

Patients randomized to Antibody 1 received a single i.v. infusion of study drug approximately 4-8 hours after surgical wound closure. For patients assigned to enoxaparin, 40 mg subcutaneous (s.c.) was administered starting approximately 12 hours after TKA surgery and then every day until the Day 10±2 venography was completed.

Patients were seen for all visits on the designated day or within the designated visit windows. The duration of hospitalization after surgery was at the investigator's discretion and according to the local medical practice. If a patient was discharged on Day 4 (prior to the Day 6±1 study visit, but after Day 3 assessments was already completed), sites collected the Day 6 assessments prior to discharge. All patients who discharged before Day 10 (±2 days) returned to the investigational site for the Day 10 visit for clinical investigation and the venography assessment.

At all visits, investigators will check for and record clinical signs and symptoms of DVT/PE, bleeding events, AEs—including ISRs—and concomitant medication changes.

### Follow-Up Period (Day 10±2 to Day 110)

After venography, patients returned to the Study Center/Physician's office for outpatient visits as indicated on the assessment schedule. Patients were followed until the study center visit on Day 110 (EoS visit).

At all visits, investigators checked for and recorded: clinical signs and symptoms of DVT/PE, bleeding events, AEs, and concomitant medication changes.

### Efficacy

The primary efficacy outcome was the composite of asymptomatic DVT as detected by unilateral ascending venography of the operated leg, confirmed symptomatic DVT, fatal and non-fatal PE, or unexplained death for which PE could not be ruled out through the Treatment Period through Day 10 (day of venography).

The secondary efficacy outcomes was the compositive of asymptomatic DVT as detected by the protocol-required unilateral venography of the operated leg at Day 10±2, confirmed symptomatic DVT, fata and non-fatal PE, or unexplained death for which PE could not be ruled-out through Day 30 and to Day 110 (EoS).

### Venography

Unilateral ascending venography of the operated leg was performed at the Day 10±2 visit (Rabinov and Paulin, 1972). Details regarding the venography procedure and image acquisition were provided in the manual for venography and event reporting which were provided separately to the study sites.

The result of the review were:
No clot
Distal clot
Proximal clot
Distal and proximal clot
Not evaluable distal but no proximal clot
Not evaluable.
   Clinical examination for DVT/PE

Patients were evaluated for signs and symptoms of DVT (e.g., swelling, localized pain, redness, heat, localized warmth) and PE (e.g., unexplained shortness of breath, chest pain that gets worse with a deep breath or coughing, or coughing up of blood) during the post-surgery in-hospital period and at all follow-up contacts after discharge.

Every suspected episode of DVT was documented by CUS or venography. If there is a suspicion of DVT, it is allowed to perform in the first instance a CUS. Only if the outcome of the CUS showed a proximal DVT of the leg, venography was not required.

Every suspected episode of PE was documented by ventilation/perfusion scintigraphy, spiral computed tomography (CT), or pulmonary angiography. If the presence of a PE was confirmed using the above techniques, the Day 10 venography did not need to be done. If a PE was not confirmed using the above techniques, the patient underwent the venography.

### Bleeding

The primary safety outcome was the combination of major bleeding and CRNM bleeding events during the Treatment Period (first study drug administration through to the Day 10 venography), Day 30, and up to the Day 110 EoS study visit (or early study discontinuation/termination).

The Investigator checked for and reported all suspected bleeding events, and additional examination was done if deemed necessary by the investigator (e.g. blood sample for hemoglobin (Hb), hematocrit (Hct), aPTT, PT, INR, and platelet count, or depending on type of bleeding endoscopy and, if applicable, the number of transfusions of packed red blood cells and transfused quantities as documented.

Other bleeding related parameters were recorded during the trial:
Hb level, Hct, and red cell count changes during the Treatment Period
Blood loss (peri-, post-operative) quantified by the routine method in each hospital
Number of transfusions of packed red cells and transfused quantities until 30 days after surgery (homologous and autologous transfusions need to be distinguished)

If the decrease in hemoglobin, the observed blood loss, or the number of transfusions exceeded what was expected following the TKA, a suspected bleeding event was be recorded.

### Pharmacokinetics

PK samples were collected at given timepoints. Instructions outlined in the Central Laboratory Manual regarding sample collection, numbering, processing and shipment were followed.

In order to better define the PK profile, the timing of the PK sample collection was altered based on emerging data. The number of samples/blood draws and total blood volume collected did not exceed those stated in the protocol. Changes to the PK Assessment timing, if any, were communicated to the sites in the dose adjustment minutes.

PK samples were obtained and evaluated in all patients at all dose levels with the exception of patients assigned to the enoxaparin arm.

Concentrations of plasma total Antibody 1 (i.e., Antibody 1 that is bound to FXI or not bound to FXI) were determined by a validated LC-MS/MS method. A detailed description of the method used to quantify the concentration of total Antibody 1 was included in the bioanalytical raw data and in the Bioanalytical Data Report. All concentrations below the LLOQ or missing data were labeled as such in the concentration data listings.

The following PK parameters were determined, where data permit, using the actual recorded sampling times and non-compartmental method(s) with Phoenix WinNonlin (Version 6.2 or higher): C0 (the concentration at the end of infusion), AUClast, AUCinf, C0/D, and AUC/D, based on the plasma concentration data. The linear trapezoidal rule will be used for AUC calculation. The terminal half-life of Antibody 1 (T_{1/2}), volume of distribution (Vss) and clearance (CL) were also estimated, if feasible, based on the data. Additional PK parameters were calculated as appropriate.

Plasma total Antibody 1 concentration data were listed by dose, patient, and visit/sampling time point. Descriptive summary statistics were provided by dose and visit/sampling time point, including the frequency (n, %) of concentrations below the LLOQ and reported as zero.

Summary statistics included mean (arithmetic and geometric), SD, coefficient of variant (CV) (arithmetic and geometric), median, minimum and maximum. Tₘₐₓ will be presented as median, minimum and maximum only. Concentrations below LLOQ were excluded from summary statistics. A geometric mean was not reported if the dataset includes zero values. PK parameters were calculated as described above and listed by dose and patient.

An assessment of dose proportionality of exposure was conducted following single-dose administrations of Antibody 1 using C₀, AUCₗₐₛₜ, and AUC_{inf}. Parameters were log transformed and analyzed using a power model: 1n(PK)=μ+β×1n(Dose). A point estimate for the slope (β), together with its 90% confidence interval (CI), was obtained from this model.

### Pharmacodynamic Assessments

PD samples were collected at the timepoints defined in the assessment schedule. Instructions for biosample processing were contained in the Central Laboratory Manual regarding sample collection, numbering, processing and shipment.

In order to better define the PD profile, the timing of the sample collection was altered based on emergent data. The number of samples/blood draws and total blood volume collected did not exceed those stated in the protocol. PD samples were obtained and evaluated in all patients at all dose levels.

Biomarkers including but not limited to the following were studied:
Free FXI— Free FXI (FXI that is not bound to Antibody 1) as measured in plasma
Total FXI— FXI that is either bound to Antibody 1 or free as measured in plasma.
aPTT calibrated for FXI deficiency
FXI coagulation activity (FXI: C) as measured in plasma

Log-transformed ratio to baseline free FXI was analyzed using a Mixed Model Repeated Measures (MMRM) approach including treatment, visit, treatment*visit interaction, log(baseline) and log(baseline)*visit interaction as effects with an unstructured variance-covariance matrix. Other models may be considered. Dose-response may be explored using appropriate contrasts. Ratio to baseline for each treatment and placebo-adjusted ratio to baseline for each Antibody 1 dose along with their associated confidence intervals will be derived after back transformation. Additionally, ratio to baseline will be compared among dose groups receiving Antibody 1. aPTT and other PD and biomarker parameters will be analyzed using the same approach. The relationship between free FXI, FXI: C and aPTT levels will be explored using graphical and regression methods.

### Immunogenicity

A ligand-binding assay was used to detect anti-Antibody 1 anti-drug antibodies (ADAs).

### Exploratory Biomarkers (Blood)

Blood samples were collected at timepoints defined in the Assessment schedule. These samples were banked for potential future analysis as deemed appropriate. Samples may be tested for, for example but not limited to, D-dimer and other exploratory markers of thrombogenesis and coagulation activity.

### Efficacy

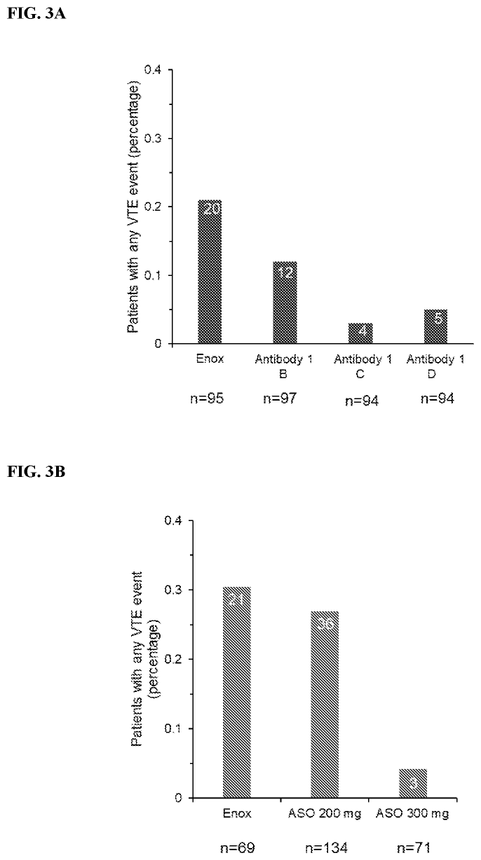

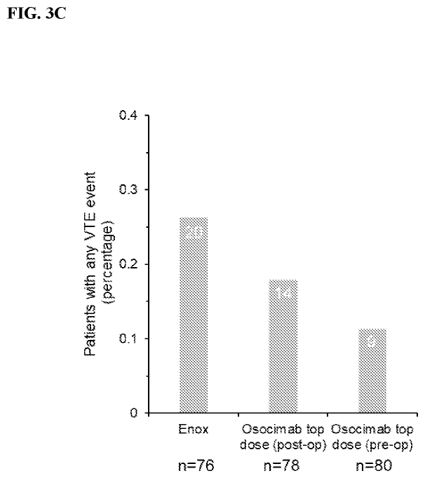

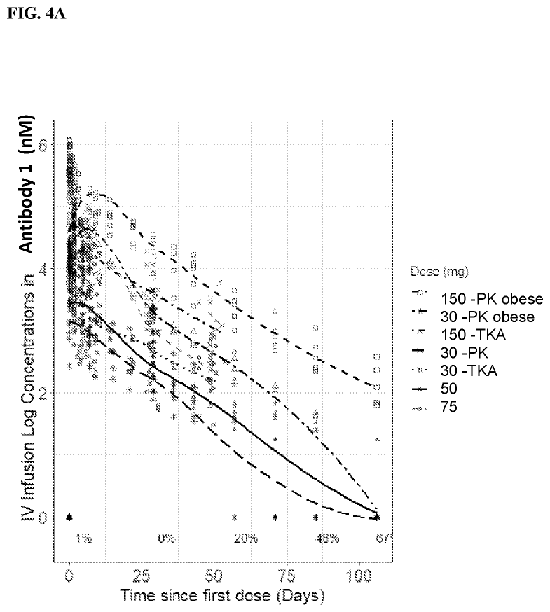

Based on assessment of venous thromboembolism (VTE) events in patients receiving Antibody 1 compared to enoxaparin following total knee arthroplasty (TKA) surgery, as described in Table 12 above, Antibody 1 was shown to be effective in preventing TKA surgery-associated VTE. Results of patients receiving doses of 30 mg (“Group B”), 75 mg (“Group C”), or 150 mg (“Group D”) of Antibody 1 or 40 mg of enoxaparin are shown in FIG. 3A. Maximum efficacy was observed in patients receiving 75 mg of Antibody 1. Results of this trial are compared to published clinical trials assessing VTE events in similar patient populations (subjects undergoing TKA surgery) using Factor XI antisense oligonucleotide (FXI-ASO) from Büller et al. NEJM (2015) (FIG. 3B) and osocimab (FOXTROT) from Weitz et al. JAMA (2020) (FIG. 3C). By comparison, Antibody 1 was found to be more efficacious in preventing TKA surgery-associated VTE than any tested dose of oscimab in the FOXTROT study, and displayed a similar efficacy as the highest dosage of FXI-ASO.

### Biomarker Analysis

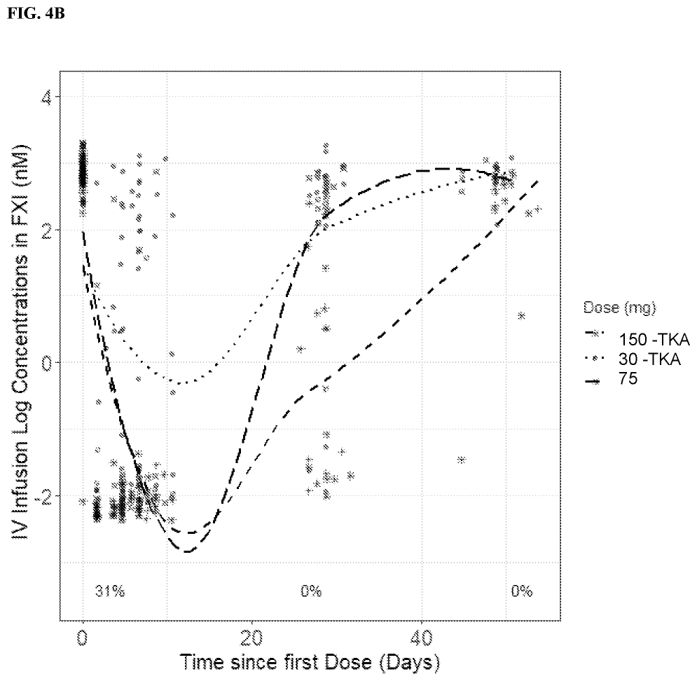

Preliminary results of this study demonstrated high efficacy of Antibody 1. As shown in FIG. 4A, peak levels of Antibody 1 in plasma demonstrated a distinct concentration difference and pattern in plasma for the TKA Trial relative to the PK/PD study conducted in healthy subjects (referred to as “PK” in the Figure). By contrast, free Factor XI observed in plasma showed a significant reduction for treated subjects up to about 57 days, as was observed across studies with Antibody 1 (FIG. 4B). For each Figure, dots represent the observed data, lines represent the smoothing “loess” curve (indicating the relationship between 2 variables), and the numbers at the bottom represent percentage of BLQ records at each time group.

### Safety

Based on analysis of serious adverse events (SAE), including major bleeding events and clinically-relevant non-major (CRNM) bleeding, Antibody 1 was safe and well-tolerated in post-operative TKA surgery patients. Patients receiving Antibody 1 were at slightly increased, although still low, risk of SAE or bleeding events compared to patients receiving enoxaparin. These risks were slightly reduced compared to those seen in patients receiving FXI-ASO or FOXTROT therapies. A quantification of SAEs from studies comparing Antibody 1, FXI-ASO, or FOXTROT to anticoagulant compounds is shown in Table 14.

TABLE 14
Safety Evaluation of Antibody 1, FXI-ASO, and FOXTROT in Patients
Undergoing TKA Surgery

| | | | FXI-ASO | | FOXTROT (Osocimab) | | |
|---|---|---|---|---|---|---|---|
| | Antibody 1 | | Büller et al. NEJM (2015) | | Weitz et al. JAMA (2020) | | |
| | Enox | Ab 1 | Enox | FXI-ASO | Enox | FOXTROT | Apixaban |
| No. patients | 103 | 309 | 69 | 205 | 102 | 585 | 100 |
| treated | | | | | | | |
| No. of SAEs | 0 | 3 (1.0%) | 0 | 4 (2%) | 6 (5.6%) | 18 (3.1%) | 1 (1.0%) |
| (%) | | | | | | | |
| No. of CRNM | 0 | 1 (0.3%) | 0 | 1 (0.5%) | 0 | 1 (0.2%) | 0 |
| Bleeds (%) | | | | | | | |
| No. of CRNM | 0 | 3 (1.0%) | 6 (8.7%) | 5 (2.4%) | 6 (5.6%) | 12 (2.1%) | 2 (2.0%) |
| Bleeds (%) | | | | | | | |

Stratification of safety data in patients receiving Antibody 1 by dosage received showed that the majority of SAEs and bleeding events occurred in group D patients, who received the highest dosage (150 mg). Two SAEs were noted in group D, with one patient experiencing a possible ileus around 4 days post-surgery and another experiencing a periprosthetic joint infection around 12 days post-surgery. The patient who experienced the periprosthetic also experienced a major bleed and a CRNM bleed. The only other SAE noted was a periprosthetic joint infection in a group C (75 mg) patient. CRNM bleeds were noted in two group B (30 mg) patients. Table 15 details safety data for patients receiving Antibody 1 therapy compared to enoxaparin.

TABLE 15
Safety Data for Patients Receiving Differing Dosasges of Anitbody 1

| | | Antibody 1 | | |
|---|---|---|---|---|
| | Enoxaparin | Group B | Group C | Group D |
| No. patients treated | 103 | 104 | 100 | 105 |
| No. of SAEs (%) | 0 | 0 | 1 (1.0%) | 2 (1.9%) |
| No. of CRNM Bleeds (%) | 0 | 0 | 0 | 1 (1.0%) |
| No. of CRNM Bleeds (%) | 0 | 2 (1.9%) | 0 | 1 (1.0%) |

Taken together, these results indicate that Antibody 1 is a safe, efficacious therapy for the prevention of VTE events in patients following TKA surgery. Optimal safety and efficacy results were seen in patients receiving the 75 mg dosage of Antibody 1 following TKA surgery.

### Example 4: Treatment of Patients with Atrial Fibrillation with Antibody 1 Compared to Rivaroxaban

### Purpose and Rationale

The purpose of this study is to evaluate the safety and tolerability of Antibody 1 compared to rivaroxaban in patients with atrial fibrillation (AF).

In a previous study conducted in healthy volunteers, Antibody 1 was safe and well tolerated, and produced robust and sustained inhibition of Factor XI and relevant prolongation of activated partial thromboplastin time (aPTT) for ≥4 weeks with doses ≥150 mg. A robust and sustained inhibition of FXI and relevant aPTT prolongation are expected to occur with the proposed dosing regimens in this study.

The study will evaluate the safety and tolerability of Antibody 1 following multiple dosing in atrial fibrillation patients at medium to high risk for stroke, as well as Antibody 1 PK and PD biomarkers. The study will assist with the dose-selection of Antibody 1 for Phase 3 in atrial fibrillation patients.

### Objectives and Endpoints

The primary objective of this study is to evaluate the effect of Antibody 1 relative to rivaroxaban on the rate of major or clinically relevant non-major (CRNM) bleeding events, with the endpoint being the time to first event of composite of International Society on Thrombosis and Haemostasis (ISTH)-defined major bleeding or CRNM bleeding events.

The secondary objectives of this study are:
to evaluate the effect of Antibody 1 relative to rivaroxaban on the total number of major or CRNM bleeding events, with the endpoint being the time to first event (an International Society on Thrombosis and Haemostasis (ISTH)-defined major bleeding event); and
to evaluate the effect of Antibody 1 relative to rivaroxaban on the rate of major or minor bleeding events, with the endpoint being the time to first event (ISTH-defined major or minor bleeding events).

Exploratory objectives of this study are:
to evaluate the effect of Antibody 1 relative to rivaroxaban on the total number of major or CRNM bleeding events, with the endpoint being the total (i.e., first and recurrent) number of ISTH-defined major or CRNM bleeding events;
to evaluate the effect of Antibody 1 relative to rivaroxaban on the total number of gastrointestinal bleeding events, with the endpoint being the total number of adjudicated ISTH-defined major or CRNM gastrointestinal bleeding events;
to evaluate the efficacy of Antibody 1 relative to rivaroxaban on the rate of stroke or systemic embolism, with the endpoint being the time to first event ischemic stroke or systemic embolic events;
to evaluate the net clinical outcome with Antibody 1 relative to rivaroxaban, with the endpoint being the time to first event of composite of ischemic stroke, systemic embolic events, major or CRNM bleeding events, all-cause mortality;
to evaluate treatment adherence of Antibody 1 relative to rivaroxaban in patients with AF, with the endpoint being the proportion of missed doses of Antibody 1 compared to the proportion of missed doses of rivaroxaban;
to assess the PK of Antibody 1 in patients with AF, with the endpoint being trough Antibody 1 plasma concentrations at indicated time points;
to assess the PD of Antibody 1 in patients with AF, with the endpoint being aPTT, free FXI, and total FXI at indicated time points;
to explore the effects of Antibody 1 on health-related quality-of-life (HRQOL) relative to rivaroxaban, with the endpoints being an EQ-5D-5L questionnaire and Anti-Clot Treatment Scale (ACTS);
to assess the incidence of immunogenicity in patients treated with Antibody 1, with the endpoints being the percentage of treated patients who develop anti-drug antibodies (ADAs) and to assess the impact of ADA development to safety, efficacy, PK and PD responses;
to assess the effects of Antibody 1 relative to rivaroxaban on additional biomarkers of thrombogenesis and coagulation, with the endpoint being exploratory coagulation parameters which may include, but not limited to, D-dimer, thrombin-activatable fibrinolysis inhibitor (TAFI) and clot lysis time; and
to perform exploratory DNA assessments to examine whether individual genetic variation in genes relating to the drug target pathway or other relevant genetic pathways confer differential responses to Antibody 1, with the endpoint being exploratory evaluation of the association between gene polymorphisms and safety, efficacy, PK, and PD response.

### Study Design

This is a randomized, double-blind, active-controlled, [dose-range finding] study. After a screening period of up to 4 weeks, patients will be randomized to 1 of 3 treatment groups (low or high dose Antibody 1 or rivaroxaban) in a 1:1:1 ratio and treated and followed for at least 12 months and until end of study. Randomization will be stratified by country and whether patients are anticoagulant naïve (Yes/No) at screening. Patients will be transitioned to a NOAC (new oral anticoagulant) and/or other standard of care therapy at the investigator's discretion at the end of study (EoS).

### Population

Approximately 900-1200 male and female patients age ≥55 with a history of atrial fibrillation or atrial flutter will be randomized in the study.

### Inclusion Criteria

The following describes the inclusion criteria for patients enrolled in the clinical study described in this example. Patients:
are male and female patients aged ≥18 years with non-valvular atrial fibrillation;
have atrial fibrillation or atrial flutter, as documented by electrocardiography;
have a CHA₂DS₂-VASc risk score ≥4 or ≥3 with at least 1 of planned concomitant use of anti-platelet medication (e.g., aspirin and/or P2Y12 inhibitor) for the duration of the trial, or CrCl≤50 ml/min by the Cockcroft-Gault equation;
have a history of prior ischemic stroke, transient ischemic attack (TIA) or non-central nervous system (CNS) systemic embolism believed to be cardioembolic in origin or has 2 or more of the following risk factors:
   Heart failure and/or left ventricular ejection fraction ≤35%
   Hypertension (defined as use of antihypertensive medication within 6 months, SBP ≥140 mmHg or DBP≥90 mmHg
   Age ≥75 years
   Diabetes mellitus (history of type 1 or type 2 or use of antidiabetic medication within 6 months);
are either anticoagulant-naïve or receiving a stable treatment of a recommended dose of a new oral anticoagulant (NOAC) over the 8 weeks prior to screening; and
when female, must be postmenopausal (≥2 years), surgically sterile, abstinent, or, if sexually active, practicing an effective method of birth control before entry and throughout study; and, for those of child-bearing potential, have a negative serum b-hCG pregnancy test at screening.

### Exclusion Criteria

The following describes the criteria that exclude patients from enrolling in the clinical study described in this example. Patients:
use of other investigational drugs within 5 half-lives prior to enrollment or until the pharmacodynamic effect has returned to baseline, whichever is longer;
have active internal bleeding;
have a history of or a condition associated with increased bleeding risk, including, but not limited to:
   Major surgical procedure or trauma within 30 days
   Clinically significant GI bleeding within 6 months
   History of intracranial, intraocular, spinal, or atraumatic intra-articular bleeding
   Chronic hemorrhagic disorder
   Known intracranial neoplasm, arteriovenous malformation, or aneurysm
have planned invasive procedure with potential for uncontrolled bleeding, including major surgery;
clinically significant mitral stenosis (value area <1.5 cm²);
mechanical heart valve;
known presence of an atrial myxoma or left ventricular thrombus;
history of left atrial appendage closure or removal;
active endocarditis;
have platelet count ≤70,000/μL at the Screening Visit;
hemoglobin <8 g/dL at the Screening Visit;
aPTT or PT >1.5× the upper limit of normal (ULN) at the Screening Visit, if patient is not on an anticoagulant;
have sustained uncontrolled hypertension SBP≥180 mmHg or DBP≥100 mmHg;
have severe disabling stroke (modified Rankin score of 4 to 5) within 3 months or any stroke within 14 days;
have transient ischemic attack (TIA) within 3 days;
have an indication for anticoagulation other than AF;
are undergoing treatment with:
   Aspirin ≥100 mg daily
   Dual antiplatelet therapy within 5 days
   Intravenous antiplatelet therapy within 5 days
   Fibrinolytics within 10 days; or
have anemia (hemoglobin <10 g/dL).

### Dosage and Administration

A dose level is randomly assigned to each patient at trial entry. In certain embodiments, patients receive 90 mg Antibody 1 subcutaneously once every month. In certain embodiments, patients receive 150 mg Antibody 1 subcutaneously once every month. In certain embodiments, patients receive 20 mg rivaroxaban orally once every evening; in certain embodiments, patients with creatinine clearance ≤50 mL/min receive 15 mg rivaroxaban once every evening.

### Efficacy and Safety Assessments

Efficacy assessments: Efficacy will be assessed by incidence of stroke (ischemic or hemorrhagic) or another systemic embolism event.

Key safety assessments: The primary endpoints based on safety are confirmed major bleeding events, clinically relevant non-major bleeding events, and total bleeding events, and/or occurrence of major cardiovascular and/or cerebral events (e.g. stroke, transient ischemic attack, systemic embolism, myocardial infarction, deep vein thrombosis, pulmonary embolism, and cardiovascular death). Adverse events will be monitored by physical examinations, monitoring of laboratory parameters in blood, ECGs, hypersensitivity reactions, injection site reactions, and the assessment of development of anti-drug antibodies.

Other assessments: Pharmacokinetics will be determined. Biomarkers that may be assessed include free Factor XI, total Factor XI, Factor XI coagulation activity, activated partial thromboplastin time, and D-dimer.

Data analysis: Exposure-response analysis across both doses of Antibody 1 relative to rivaroxaban will be analyzed for the cumulative incidence of major and clinically relevant non-major bleeding. Safety data, including bleeding events, will be analyzed as appropriate. [Assuming a control group annualized event rate of 14.2% based on ROCKET-AF and 10% annualized drop-out rate, a total sample size of 800 subjects would provide 80% power to detect an HR=0.60 at 1-sided alpha of 0.025. A total sample size of 1100 subjects would provide 80% power for an HR=0.65. These numbers assumed 1:1 allocation between test: control, 12 month enrollment and 12 months follow-up on last patient enrolled]

Interim analyses: Interim analyses may be incorporated for sample resizing or study duration extension, or stopping dose or study for findings of early efficacy or futility.

### Clinical Endpoints

### Bleeding

All suspected bleeding events either reported by the subject or observed by the Investigator should be recorded.

Overt bleeding events will be adjudicated by an independent and blinded CEC. The CEC will classify bleeding events in accordance with the International Society on Thrombosis and Haemostasis (ISTH) definitions and guidance (Kaatz et al 2015).

The details of all reported bleeding events will be submitted to the CEC as described in the endpoint reporting guidelines. These details may include, but are not limited to,
Location of the bleeding
Duration of the bleeding
Treatment of the bleeding event including notes or summaries of recommendations from a healthcare professional from whom medical treatment was obtained such as otolaryngology consults for ear, nose, or throat bleeds; urology consults for hematuria or urogenital tract bleeds; surgical consults for skin, soft tissue, or internal bleeds; gynecology consults for uterine or vaginal bleeds; neurology or neurosurgical consults for intracranial bleeds; or ophthalmology consults for ocular bleeds
Number of blood product transfusions
Magnitude of the bleeding (e.g. size of skin or subcutaneous hematoma)
Hemoglobin (Hb) levels at the time of the bleeding event, lowest value, pre- and post-transfusion values, and after resolution of the bleeding event
Any diagnostic tests done to evaluate the bleeding such as endoscopy for gastrointestinal (GI) bleeds
Any diagnostic imaging, e.g., x-ray, computed tomography (CT), magnetic resonance imaging (MRI), or ultrasound, performed to evaluate the bleeding
Any other information that could be of help to the CEC in adjudicating the bleeding event.

Endpoint Reporting Guidelines will provide detailed instructions for sites on the documentation, data collection, and reporting required for each suspected bleeding event.

### Efficacy

The independent and blinded CEC will adjudicate and classify the following events:
Ischemic stroke
Transient ischemic attack (TIA)
Non-CNS (systemic) arterial embolic events
Myocardial infarction (MI)
Death

Adjudicated results will be used for the final analysis.

### Other Assessments

### Pharmacokinetics

Blood samples for PK will be collected before administration of study drug on study visits as defined in the Assessment Schedule (as shown in Table 16 and Table 17 below). Concentrations of plasma total Antibody 1 (i.e., Antibody 1 that is bound to FXI or not bound to FXI) will be determined by a validated LC-MS/MS method. A detailed description of the method used to quantify the concentration of total Antibody 1 will be included in the bioanalytical raw data and in the Bioanalytical Data Report. All concentrations below the LLOQ or missing data will be labeled as such in the concentration data listings.

### Pharmacodynamic Assessments

PD samples will be collected at the timepoints defined in the assessment schedule. Instructions for bio-sample processing will be contained in the Central Laboratory Manual regarding sample collection, numbering, processing, and shipment.

PD Biomarkers including but not limited to the following may be studied:
Free FXI— FXI that is not bound to Antibody 1 will be measured in plasma;
Total FXI— FXI that is either bound to Antibody 1 or free will be measured in plasma;
aPTT.

Additionally, FXI coagulation activity (FXI:C) may be collected from a subset of patients enrolled at selected sites, based on the site's access to appropriate −70° C./−80° C. freezers. FXI: C will be measured in plasma.

### Immunogenicity

An immunoassay-based method will be used to detect anti-Antibody 1 anti-drug antibodies (ADAs). The analytical method will be described in detail in the IG Bioanalytical Data Report. IG samples will be collected before administration of study drug on study visits as defined in the Assessment schedule (Tables 16-17).

TABLE 16
Assessment Schedule (Year 1)

| | Screening | Treatment Period (Year 1) | | | | | | | | | | | | | |
|---|---|---|---|---|---|---|---|---|---|---|---|---|---|---|---|
| | Day | | | | | Month | | | | | | | | | |
| Period | −30 to −1 | 1 | 8 | 30 | 60 | 3 | 4 | 5 | 6 | 7 | 8 | 9 | 1 | 11 | 12 |
| Visit Window | | | ±2 | ±3 | ±3 | ±5 | ±5 | ±5 | ±5 | ±5 | ±5 | ±5 | ±5 | ±5 | ±5 |
| (days) | | | | | | | | | | | | | | | |
| Informed | X | | | | | | | | | | | | | | |
| consent/Genetic | | | | | | | | | | | | | | | |
| Consent¹ | | | | | | | | | | | | | | | |
| Inclusion/Exclusion | X | X | | | | | | | | | | | | | |
| criteria | | | | | | | | | | | | | | | |
| Demography/ | X | | | | | | | | | | | | | | |
| Medical History | | | | | | | | | | | | | | | |
| Physical Exam | S | | | | | | | | | | | | | | |
| Height and Weight | X | | | | | | | | | | | | | | |
| Vital Signs | X | X | | X | | | | | X | | | | | | X |
| 12-Lead ECG | X | | | | | | | | | | | | | | |
| HIV, Hepatitis B, | S | | | | | | | | | | | | | | |
| Hepatits C | | | | | | | | | | | | | | | |
| Serum Pregnancy | X | | | | | | | | | | | | | | |
| and FSH | | | | | | | | | | | | | | | |
| Urine Pregnancy- | | S | | S | | S | | | S | | | S | | | S |
| WOCBP only | | | | | | | | | | | | | | | |
| Hematology | X | X | | | | X | | | X | | | X | | | X |

| | | | | | | | | | | | | | | | |
|---|---|---|---|---|---|---|---|---|---|---|---|---|---|---|---|
| Chemistry | X | | | | | | | | X | | | | | | X |
| Urine Dipstick | X | | | | | | | | | | | | | | |
| PT/INR, aPTT | X | X | | X | | X | | | X | | | X | | | X |
| PK² and Free | | X | | | | X | | | X | | | X | | | X |
| FXI/Total FXI² | | | | | | | | | | | | | | | |
| Antidrug | | X | | | | X | | | X | | | X | | | X |
| Antibodies (ADA)² | | | | | | | | | | | | | | | |
| Factor XI | | X | | | | X | | | X | | | X | | | X |
| Coagulation | | | | | | | | | | | | | | | |
| Activity | | | | | | | | | | | | | | | |
| Exploratory | | X | | | | | | | | | | | | | X |
| Biomarkers | | | | | | | | | | | | | | | |
| Genetic Sample¹ | | X | | | | | | | | | | | | | |
| EQ-5D-5L and | | X | | | | | | | X | | | | | | X |
| ACTS PROs³ | | | | | | | | | | | | | | | |
| Telephone Call | | | X | | | | | | | | | | | | |
| Visit Only | | | | | | | | | | | | | | | |
| Study Drug S.C. | | X | | X | X | X | X | X | X | X | X | X | X | X | X |
| Administration⁴ | | | | | | | | | | | | | | | |
| Study Drug P.O. | | | | X | X | X | X | X | X | X | X | X | X | X | X |
| Administation⁵ | | | | | | | | | | | | | | | |
| Injection Site | | X | | X | X | X | X | X | X | X | X | X | X | X | X |
| Insepction⁴ | | | | | | | | | | | | | | | |

| | |
|---|---|
| Concomitant | X |
| Medicaitons | |
| AE Assessment | X |

S- results are documented in source documents only
¹The genetic informed consent is for an optional sub-study; the genetic sample can be collected at any time after the optional genetic informed consent is obtained
²Only collected from patients assigned to Antibody 1
³May be collected in a subset of patients
⁴Patients assigned to Antibody 1 only; Dose can be administered at in-clinic or in-home visits by medically qualified, un-blinded study staff or designees as locally permitted. If administered in-home, assessments for concomitant medications, AEs, and other changes in health status will be conducted by sites via telephone or video call as locally permitted
⁵Patients assigned to rivaroxaban only; Accountability can be assessed at in-clinic visit or via virtual (e.g. video call) visit as appropriate and locally permitted

TABLE 17
Assessment Schedule (Year 2)

| | Period | | | | | | | | | | | | | | EoS |
|---|---|---|---|---|---|---|---|---|---|---|---|---|---|---|---|
| | Treatment Period (Year 1) | | | | | | | | | | | | | | 4-wk |
| | Month | | | | | | | | | | | | | | post- |
| | 13 | 14 | 15 | 16 | 6 | 18 | 19 | 20 | 21 | 22 | 23 | 24 | | | transition |
| | Visit Window (days) | | | | | | | | | | | | PSDD | EoT | visit/ |
| | ±5 | ±5 | ±5 | ±5 | ±5 | ±5 | ±5 | ±5 | ±5 | ±5 | ±5 | ±5 | Visit | Visit | call |
| Physical | | | | | | | | | | | | | S | S | |
| Exam | | | | | | | | | | | | | | | |
| Vital Signs | | | | | | X | | | | | | X | X | X | |
| 12-lead ECG | | | | | | | | | | | | | X | X | |
| Urine- | | | S | | | S | | | S | | | S | S | S | |
| Pregnancy - | | | | | | | | | | | | | | | |
| WOCBP | | | | | | | | | | | | | | | |
| only | | | | | | | | | | | | | | | |
| Hematology | | | X | | | X | | | X | | | X | X | X | |
| Chemistry | | | | | | X | | | | | | X | | | |
| PT/INR, | | | X | | | X | | | X | | | X | X | X | |
| aPTT | | | | | | | | | | | | | | | |
| PK² and Free | | | X | | | X | | | X | | | X | X | X | |
| FX/FXI2 | | | | | | | | | | | | | | | |
| Anti drug | | | X | | | X | | | X | | | X | X | X | |
| antibodies | | | | | | | | | | | | | | | |
| (ADA)² | | | | | | | | | | | | | | | |
| Factor XI | | | X | | | X | | | X | | | X | X | X | |
| Coagulation | | | | | | | | | | | | | | | |
| Activity³ | | | | | | | | | | | | | | | |
| Exploratory | | | | | | | | | | | | X | | X | |
| biomarkers³ | | | | | | | | | | | | | | | |
| EQ-5D-5L | | | | | | X | | | | | | X | | | |
| and ACTS | | | | | | | | | | | | | | | |
| PROs³ | | | | | | | | | | | | | | | |
| Study drug | X | X | X | X | X | X | X | X | X | X | X | X | | | |
| s.c. | | | | | | | | | | | | | | | |
| administration⁴ | | | | | | | | | | | | | | | |
| Study drug | X | X | X | X | X | X | X | X | X | X | X | X | X | X | |
| p.o. | | | | | | | | | | | | | | | |
| accountability⁵ | | | | | | | | | | | | | | | |
| Injection site | X | X | X | X | X | X | X | X | X | X | X | X | X | X | |
| inspection⁴ | | | | | | | | | | | | | | | |

| | | | | |
|---|---|---|---|---|
| Concomitant | X | X | X | X |
| medications | | | | |
| AE | X | X | X | X |
| assessment | | | | |

S = results are documented in source documents only; permanent study drug discontinuation;
EoT = end of treatment period;
EoS = end of study
¹Subsequent treatment years will follow the same assessment schedule as Year 2
²Only collected from patients assigned to Antibody 1
³May be collected in a subset of patient
⁴Patients assigned to Antibody 1 only; dose can be administered at in-clinic or in-home visits by medically qualified, un-blinded study staff or designess as locally permitted. If administered in-home home; assessments for concomitant medications, AEs, and other changes in health status will be conducted by sites via telephone or video call as locally permitted
⁵Patients assigned to rivaroxaban only; Accountability can be assessed at in-clinic visit or via virtual (e.g., video call) visit as appropriate and locally permitted

### Additional Biomarkers (Blood)

Additional blood samples will be collected at timepoints defined in the Assessment schedule and banked for potential future exploratory analysis (Table 16).

Additional biomarkers may include, but are not necessarily limited to:
D-dimer
Thrombin activable fibrinolysis inhibitor (TAFI) activity
Clot lysis time

The list may be changed or expanded further, as it is recognized that more relevant or novel biomarkers may be discovered during the conduct of the study. This may be conducted in a subset of patients.

### Health-Related Quality of Life (HRQOL)

Two patient-reported outcomes (PRO) instruments will be used in this study:
The EQ-5D-5L questionnaire will be used to collect information revealing the impact of the disease and its treatment on the patient's physical, emotional and social well-being. The EQ-5D-5L is a self-administered, validated instrument that measures generic health-related quality of life across 5 dimensions: mobility, self-care, usual activities, pain/discomfort and anxiety/depression. For each dimension, there are five levels of response: In addition, the measure contains a visual analogue scale (VAS) scale that measures the respondent's overall health on a 0-100 scale. This instrument has been widely used in clinical trials across a range of clinical conditions and among the general population (Berg et al 2010).
The Anti-Clot Treatment Scale (ACTS) is a 15-item, validated PRO instrument that assesses patient satisfaction with anticoagulant treatment based on two domains, burden and benefits (Cano et al 2012).

Both PROs will be administered electronically at the timepoints noted in the Assessment schedule. This may be conducted in a subset of patients.

### Study Completion

At the EoT visit, all study drug supplies must be collected from the patient and the Investigator must begin plans to transition the patient to SoC anticoagulation therapy according to the guidelines in Table 18, unless the patient has a contraindication to anticoagulation therapy.

TABLE 18
End of Study Transition Guidelines

| Transitioning to | Instructions |
|---|---|
| Direct oral | Patients should initiate oral DOAC on the same |
| anticoagulant | day of the EOT visit. |
| (DOAC) | |
| Vitamin K antagonist | Patients should be given a supply of VKA and |
| (VKA) | instructed to begin therapy 3 to 5 days before the |
| | EoT visit. At the EOT visit, measure PT.INR and |
| | repeat as frequently as necessary until the PT/INR |
| | is therapeutic |

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

What is claimed is:

1. A vial comprising a drug delivery formulation comprising:
(a) a therapeutically effective amount of an isolated anti-Factor XI (FXI) and/or anti-activated Factor XI (FXIa) antibody, or antigen-binding fragment thereof;
(b) a histidine buffer;
(c) a sugar or sugar alcohol; and
(d) a polysorbate,
at pH 5.0 to 6.0,
wherein the vial comprises an overfill for complete withdrawal of a therapeutically effective amount of the anti-FXI and/or anti-FXIa antibody or the antigen-binding fragment thereof.

2. The vial of claim 1, wherein the therapeutically effective amount of the isolated anti-FXI and/or anti-FXIa antibody or antigen-binding fragment thereof is at a concentration between 120 mg/ml and 180 mg/ml.

3. The vial of claim 1 or 2, wherein the therapeutically effective amount of the isolated anti-FXI and/or anti-FXIa antibody or antigen-binding fragment thereof is at a concentration of about 150 mg/ml.

4. The vial of any one of claims 1-3, wherein the histidine buffer comprises a histidine and a histidine salt.

5. The vial of claim 4, wherein the histidine is L-histidine.

6. The vial of claim 4 or 5, wherein the histidine salt is histidine HCl monohydrate.

7. The vial of any one of claims 4-6, wherein the histidine buffer is at a concentration between 10 mM and 30 mM.

8. The vial of claim 7, wherein the histidine buffer is at a concentration of about 20 mM.

9. The vial of any one of claims 1-8, wherein the sugar or sugar alcohol is a disaccharide.

10. The vial of claim 9, wherein the disaccharide is sucrose.

11. The vial of claim 10, wherein the sucrose is at a concentration between 170 mM to 270 mM.

12. The vial of claim 11, wherein the sucrose is at a concentration of about 220 mM.

13. The vial of any one of claims 1-12, wherein the polysorbate is polysorbate 20.

14. The vial of claim 13, wherein the polysorbate 20 is at a concentration between 0.02% (v/v) to 0.06% (v/v).

15. The vial of claim 14, wherein the polysorbate 20 is at a concentration of about 0.04% (v/v).

16. The vial of any one of claims 1-15, wherein the pH is 5.3 to 5.7.

17. The vial of any one of claims 1-16, wherein the pH is about 5.5.

18. The vial of any one of claims 1-17, wherein the overfill comprises between 10% (v/v) and 30% (v/v) of the drug delivery formulation, optionally wherein the vial comprises 1.1 mL to 1.3 mL of the drug delivery formulation.

19. The vial of claim 18, wherein the overfill comprises about 20% (v/v) of the drug delivery formulation, optionally wherein the vial comprises about 1.2 mL of the drug delivery formulation.

20. The vial of any one of claims 1-19, wherein the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

21. The vial of any one of claims 1-20, wherein the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37; and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37; and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

22. The vial of any one of claims 1-21, wherein the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

23. The vial of any one of claims 1-22, wherein the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

24. The vial of any one of claims 1-23, wherein the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

25. The vial of any one of claims 1-24, wherein the antibody comprises a heavy chain comprising an amino acid sequence of SEQ ID NO: 31 and a light chain comprising an amino acid sequence of SEQ ID NO: 41.

26. The vial of any one of claims 1-25, wherein the antibody is a human monoclonal antibody.

27. The vial of claim 26, wherein the antibody is a human IgG1 isotype.

28. The vial of claim 26 or 27, wherein the antibody comprises D265A and P329A substitutions in the Fc domain, optionally wherein 120 mg to 180 mg is the therapeutically effective amount of the anti-Factor XI (FXI) and/or anti-activated Factor XI (FXIa) antibody or antigen-binding fragment thereof, for administration to a subject.

29. A vial comprising a drug delivery formulation comprising:
(a) a therapeutically effective amount of an isolated anti-Factor XI (FXI) and/or anti-activated Factor XI (FXIa) antibody, or antigen-binding fragment thereof at a concentration of about 150 mg;
(b) a histidine buffer at a concentration of about 20 mM;
(c) sucrose at a concentration of about 220 mM; and
(d) polysorbate-20 at a concentration of about 0.04% (v/v),
at pH 5.5,
wherein the vial comprises an overfill for complete withdrawal of a therapeutically effective amount of the anti-FXI and/or anti-FXIa antibody or the antigen-binding fragment thereof.

30. An intravenous drug delivery formulation comprising:
(a) a therapeutically effective amount of an isolated anti-FXI and/or anti-FXIa antibody or antigen-binding fragment thereof;
(b) a histidine buffer;
(c) a sugar or sugar alcohol;
(d) a polysorbate, and
(e) a diluent
at pH 5.0 to 6.0,
wherein the diluent is a solution comprising a second sugar and water.

31. The intravenous drug delivery formulation of claim 30, wherein the therapeutically effective amount of the isolated anti-FXI and/or anti-FXIa antibody or antigen-binding fragment thereof is at a concentration between 1.20 mg/ml and 1.80 mg/ml.

32. The intravenous drug delivery formulation of claim 30 or 31, wherein the therapeutically effective amount of the isolated anti-FXI and/or anti-FXIa antibody or antigen-binding fragment thereof is at a concentration of about 1.50 mg/ml.

33. The intravenous drug delivery formulation of any one of claims 30-32, wherein the histidine buffer comprises a histidine and a histidine salt.

34. The intravenous drug delivery formulation of claim 33, wherein the histidine is L-histidine.

35. The intravenous drug delivery formulation of claim 33 or 34, wherein the histidine salt is histidine HCl monohydrate.

36. The intravenous drug delivery formulation of any one of claims 30-35, wherein the histidine buffer is at a concentration between 0.10 mM and 0.30 mM.

37. The intravenous drug delivery formulation of any one of claims 30-36, wherein the histidine buffer is at a concentration of about 0.20 mM.

38. The intravenous drug delivery formulation of any one of claims 30-37, wherein the sugar or sugar alcohol is a disaccharide.

39. The intravenous drug delivery formulation of claim 38, wherein the disaccharide is sucrose.

40. The intravenous drug delivery formulation of claim 39, wherein the sucrose is at a concentration between 1.70 mM to 2.70 mM.

41. The intravenous drug delivery formulation of claim 40, wherein the sucrose is at a concentration of about 2.20 mM.

42. The intravenous drug delivery formulation of any one of claims 30-41, wherein the polysorbate is polysorbate 20.

43. The intravenous drug delivery formulation of claim 42, wherein the polysorbate 20 is at a concentration of less than 0.001% (v/v).

44. The intravenous drug delivery formulation of claim 43, wherein the polysorbate 20 is at a concentration of about 0.0004% (v/v).

45. The intravenous drug delivery formulation of any one of claims 30-44, wherein the pH is 5.3 to 5.7.

46. The intravenous drug delivery formulation of any one of claims 30-45, wherein the pH is about 5.5.

47. The intravenous drug delivery formulation of any one of claims 30-46, wherein the second sugar in the diluent is a monosaccharide.

48. The intravenous drug delivery formulation of claim 47, wherein the monosaccharide is dextrose.

49. The intravenous drug delivery formulation of claim 48, wherein the dextrose is at a concentration between 2.5% (v/v) and 7.5% (v/v).

50. The intravenous drug delivery formulation of claim 48 or 49, wherein the dextrose is at a concentration of about 5% (v/v).

51. The intravenous drug delivery formulation of any one of claims 30-50, wherein the antibody or antigen-binding fragment thereof comprises a heavy chain variable region (VH) comprising complementary determining regions HCDR1, HCDR2, and HCDR3 in SEQ ID NO: 9 or 29; and a light chain variable region (VL) comprising complementary determining regions LCDR1, LCDR2, LCDR3 in SEQ ID NO: 19 or 39.

52. The intravenous drug delivery formulation of any one of claims 30-51, wherein the antibody or antigen-binding fragment thereof comprises:
i. a heavy chain variable region CDR1 of SEQ ID NO: 23; a heavy chain variable region CDR2 of SEQ ID NO: 24; a heavy chain variable region CDR3 of SEQ ID NO: 25; a light chain variable region CDR1 of SEQ ID NO: 33; a light chain variable region CDR2 of SEQ ID NO: 34; and a light chain variable region CDR3 of SEQ ID NO: 35;
ii. a heavy chain variable region CDR1 of SEQ ID NO: 26; a heavy chain variable region CDR2 of SEQ ID NO: 27; a heavy chain variable region CDR3 of SEQ ID NO: 28; a light chain variable region CDR1 of SEQ ID NO: 36; a light chain variable region CDR2 of SEQ ID NO: 37; and a light chain variable region CDR3 of SEQ ID NO: 38;
iii. a heavy chain variable region CDR1 of SEQ ID NO: 43; a heavy chain variable region CDR2 of SEQ ID NO: 44; a heavy chain variable region CDR3 of SEQ ID NO: 45; a light chain variable region CDR1 of SEQ ID NO: 47; a light chain variable region CDR2 of SEQ ID NO: 37; and a light chain variable region CDR3 of SEQ ID NO: 15; or
iv. a heavy chain variable region CDR1 of SEQ ID NO: 46; a heavy chain variable region CDR2 of SEQ ID NO: 4; a heavy chain variable region CDR3 of SEQ ID NO: 5; a light chain variable region CDR1 of SEQ ID NO: 33; a light chain variable region CDR2 of SEQ ID NO: 14; and a light chain variable region CDR3 of SEQ ID NO: 15.

53. The intravenous drug delivery formulation of any one of claims 30-52, wherein the antibody or antigen-binding fragment thereof comprises a heavy chain variable region (VH) selected from the group consisting of SEQ ID NO: 9, 29, and a VH with 90% identity thereto; and a light chain variable region (VL) selected from the group consisting of SEQ ID NO: 19, 39, and a VL with 90% identity thereto.

54. The intravenous drug delivery formulation of any one of claims 30-53, wherein the antibody or antigen-binding fragment thereof comprises a heavy chain variable region (VH) selected from the group consisting of SEQ ID NO: 9 and 29; and a light chain variable region (VL) selected from the group consisting of SEQ ID NO: 19 and 39.

55. The intravenous drug delivery formulation of any one of claims 30-54, wherein the antibody comprises a heavy chain comprising an amino acid sequence selected from the group consisting of SEQ ID NO: 31, 11, and a heavy chain with 90% identity thereto; and a light chain comprising an amino acid sequence selected from the group consisting of SEQ ID NO: 41, 21, and a light chain with 90% identity thereto.

56. The intravenous drug delivery formulation of any one of claims 30-55, wherein the antibody comprises a heavy chain comprising an amino acid sequence of SEQ ID NO: 31 and a light chain comprising an amino acid sequence of SEQ ID NO: 41.

57. The intravenous drug delivery formulation of any one of claims 30-56, wherein the antibody is a human monoclonal antibody.

58. The intravenous drug delivery formulation of claim 57, wherein the antibody is a human IgG1 isotype.

59. The intravenous drug delivery formulation of claim 57 or 58, wherein the antibody comprises D265A and P329A substitutions in the Fc domain, optionally wherein 120 mg to 180 mg is the therapeutically effective amount of the anti-Factor XI (FXI) and/or anti-activated Factor XI (FXIa) antibody or antigen-binding fragment thereof for administration to a subject.

60. An intravenous drug delivery formulation comprising:
(a) a therapeutically effective amount of an isolated anti-FXI and/or anti-FXIa antibody or antigen-binding fragment thereof at a concentration of about 1.5 mg;
(b) a histidine buffer at a concentration of about 0.20 mM;
(c) sucrose at a concentration of about 2.20 mM;
(d) a polysorbate-20 at a concentration of about 0.0004% (v/v), and
(e) a diluent
at pH 5.5,
wherein the diluent is dextrose 5% in water (D5W).

61. A method of treating a subject afflicted with or at risk of developing a thromboembolic disorder, the method comprising administering a therapeutically effective amount of the drug delivery formulation present in the vial of any one of claims 1-28 or the intravenous drug delivery formulation of any one of claims 30-59 to the subject in need thereof.

62. The method of claim 61, wherein the thromboembolic disorder is selected from the group consisting of atrial fibrillation or atrial flutter, transient ischemic attack, ischemic stroke, thromboembolic stroke, hemorrhagic stroke, venous thromboembolism (VTE), pediatric VTE, systemic embolism, non-central nervous systemic embolism, myocardial infarction, deep vein thrombosis, Severe Protein S deficiency, cerebrovascular accident, and cancer.

63. The method of claim 61 or 62, wherein the drug delivery formulation present in the vial or the intravenous drug delivery formulation is administered monthly.

64. The method of any one of claims 61-63, wherein the drug delivery formulation present in the vial or the intravenous drug delivery formulation is administered at a dose selected from the group consisting of about 30 mg, about 60 mg, about 90 mg, about 120 mg, about 150 mg, and about 180 mg.

65. The method of any one of claims 61-64, wherein the drug delivery formulation present in the vial is administered at a dose of about 120 mg.

66. The method of any one of claims 61-64, wherein the drug delivery formulation present in the vial is administered at a dose of about 150 mg.

67. The method of any one of claims 61-66, wherein the drug delivery formulation present in the vial is administered subcutaneously.

68. The method of any one of claims 61-67, wherein the thromboembolic disorder is atrial fibrillation or atrial flutter.

69. The method of claim 68, wherein the atrial fibrillation or atrial flutter is paroxysmal atrial fibrillation (PAF).

70. The method of any one of claims 61-69, wherein the drug delivery formulation present in the vial is administered once a month for a period of three months.

71. The method of any one of claims 61-70, wherein the subject is at low risk of stroke.

72. The method of claim 71, wherein the subject has a CHA₂DS₂VASc risk score of 0 to 1.

73. The method of any one of claims 61-70, wherein the subject is at moderate risk of stroke.

74. The method of any one of claims 61-70, wherein the subject is at high risk of stroke.

75. The method of claim 74, wherein the subject has a CHA₂DS₂VASc risk score of ≥2 for male subjects and >3 for female subjects.

76. The method of any one of claims 61-75, the method further comprising evaluating efficacy of the drug delivery formulation present in the vial by measuring inhibition of Factor XI at the trough after the third dose of the drug delivery formulation.

77. The method of any one of claims 61-76, the method further comprising evaluating efficacy of the drug delivery formulation present in the vial by assessing one or more biomarkers selected from the list consisting of free Factor XI, total Factor XI, Factor XI coagulation activity, activated partial thromboplastin time, and D-dimer.

78. The method of any one of claims 61-77, the method further comprising evaluating adverse events to the drug delivery formulation present in the vial by measuring bleeding events or the presence of anti-drug antibodies.

79. The method of claim 78, the method further comprising applying one or more of the following to the patient experiencing an adverse event, wherein the adverse event is a bleeding event: (i) fluid replacement using colloids, crystalloids, human plasma or plasma proteins such as albumin; (ii) transfusion with packed red blood or whole blood; or (iii) administration of fresh frozen plasma (FFP), prothrombin complex concentrates (PCC), activated PCC (APCC), such as, factor VIII inhibitor, and/or recombinant, activated factor VII.

80. A method of treating a subject afflicted with or at risk of developing a thromboembolic disorder and who is undergoing a surgical procedure, the method comprising administering the intravenous drug delivery formulation of any one of claims 30-59 to a subject in need thereof, wherein the subject is administered the intravenous drug delivery formulation on the same day as the surgical procedure.

81. The method of claim 80, wherein the surgical procedure is selected from the group consisting of knee replacement surgery, hip replacement surgery, orthopedic surgery, pacemaker installation, catheter installation, thoracic surgery, and abdominal surgery.

82. The method of claim 80 or 81, wherein the intravenous drug delivery formulation is administered monthly.

83. The method of any one of claims 80-82, wherein the intravenous drug delivery formulation is administered at a dose selected from the group consisting of about 30 mg, about 60 mg, about 90 mg, about 120 mg, about 150 mg, and about 180 mg.

84. The method of any one of claims 80-83, wherein the intravenous drug delivery formulation is administered at a dose of about 30 mg.

85. The method of any one of claims 80-83, wherein the intravenous drug delivery formulation is administered at a dose of about 60 mg.

86. The method of any one of claims 80-83, wherein the intravenous drug delivery formulation is administered at a dose of about 150 mg.

87. The method of any one of claims 80-82, wherein the intravenous drug delivery formulation is administered at a dose of about 75 mg.

88. The method of any one of claims 80-87, wherein the intravenous drug delivery formulation is administered approximately 4-8 hours after surgery.

89. A method of treating a subject afflicted with or at risk of developing a thromboembolic disorder, wherein the subject is receiving non-steroidal anti-inflammatory drugs (NSAIDs), the method comprising administering a therapeutically effective amount of the drug delivery formulation present in the vial of any one of claims 1-28 or the intravenous drug delivery formulation of any one of claims 30-59 in combination with a proton-pump inhibitor to the subject in need thereof.

90. The method of claim 89, wherein the drug delivery formulation present in the vial or the intravenous drug delivery formulation is administered monthly.

91. The method of claim 89 or 90, wherein the drug delivery formulation present in the vial or the intravenous drug delivery formulation is administered at a dose selected from the group consisting of about 30 mg, about 60 mg, about 90 mg, about 120 mg, about 150 mg, and about 180 mg.

92. The method of claim 88 or 89, wherein the drug delivery formulation present in the vial or the intravenous drug delivery formulation is administered at a dose of about 75 mg.

93. The method of any one of claims 89-91, wherein the drug delivery formulation present in the vial is administered subcutaneously.
